(19) United States
(12) Patent Application Publication
(10) Pub. No.: US 2004/0142978 A1
(43) Pub. Date: Jul. 22, 2004

(54) Aminocyanopyridine inhibitors of mitogen activated protein kinase-activated protein kinase-2

(75) Inventors: David R. Anderson, Lake St. Louis, MO (US); Nathan W. Stehle, Grafton, WI (US); Stephen A. Kolodziej, Ballwin, MO (US); Emily J. Reinhard, Ridgewood, NJ (US); Len F. Lee, St. Charles, MO (US)

Correspondence Address: Charles E. Dunlap, P.O. Box 11070, Columbia, SC, 29211-1070, US

(73) Assignee: Pharmacia Corporation, St. Louis, MO (US)

(21) Appl. No.: 10/729,139

(22) Filed: Dec. 5, 2003

Related U.S. Application Data
(60) Provisional application No. 60/432,843, filed on Dec. 12, 2002.

(51) Int. Cl. C07D 213/84; A61K 31/44

(52) U.S. Cl. 514/344; 546/289; 514/340; 546/268.1

(57) ABSTRACT

Aminocyanopyridine compounds are described which can inhibit mitogen activated protein kinase-activated protein kinase-2. Pharmaceutical compositions and kits that contain these compounds are also described.

### CROSS REFERENCE TO RELATED PATENTS AND PATENT APPLICATIONS

This application claims the benefit of U.S. Provisional Patent Application Serial No. 60/432,843, filed Dec. 12, 2002, which is incorporated herein by reference in its entirety. This application is related to a commonly assigned and copending application having the title “Method of using aminocyanopyridine compounds as mitogen activated protein kinase-activated protein kinase-2 inhibitors” (and having Provisional Application Serial No. 60/432,807, which was filed on the same date as the present application.

### BACKGROUND OF THE INVENTION

(1) Field of the Invention

The present invention relates to certain aminocyanopyridine compounds, and in particular, to aminocyanopyridine compounds which are capable of inhibiting mitogen-activated protein kinase-activated protein kinase-2 (MAPKAP kinase-2, or MK-2), and to compositions and kits that contain such compounds.

(2) Description of the Related Art

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif with the sequence Thr-X-Tyr by mitogen-activated protein kinase kinases (MAPKKs). In higher eukaryotes, the physiological role of MAPK signaling has been correlated with cellular events such as proliferation, oncogenesis, development and differentiation. Accordingly, the ability to regulate signal transduction via these pathways could lead to the development of treatments and preventive therapies for human diseases associated with MAPK signaling, such as inflammatory diseases, autoimmune diseases and cancer.

In mammalian cells, three parallel MAPK pathways have been described. The best characterized pathway leads to the activation of the extracellular-signal-regulated kinase (ERK). Less well understood are the signal transduction pathways leading to the activation of the cJun N-terminal kinase (JNK) and the p38 MAPK. See, e.g., Davis, Trends Biochem. Sci. 19:470-473 (1994); Cano, et al., Trends Biochem. Sci. 20:117-122(1995).

The p38 MAPK pathway is potentially activated by a wide variety of stresses and cellular insults. These stresses and cellular insults include heat shock, UV irradiation, inflammatory cytokines (such as TNF and IL-1), tunicamycin, chemotherapeutic drugs (i.e., cisplatinum), anisomycin, sorbitol/hyperosmolarity, gamma irradiation, sodium arsenite, and ischaemia. See, Ono, K., et al, Cellular Signalling 12, 1-13 (2000). Activation of the p38 pathway is involved in (1) production of proinflammatory cytokines, such as TNF-α; (2) induction of enzymes, such as Cox-2; (3) expression of an intracellular enzyme, such as iNOS, which plays an important role in the regulation of oxidation; (4) induction of adherent proteins, such as VCAM-1 and many other inflammatory-related molecules. Furthermore, the p38 pathway functions as a regulator in the proliferation and differentiation of cells of the immune system. See, Ono, K., et al., Id. at 7.

The p38 kinase is an upstream kinase of mitogen-activated protein kinase-activated protein kinase-2 (MAPKAP kinase-2 or MK-2). (See, Freshney, N. W., et al., J. Cell, 78:1039-1049 (1994)). MK-2 is a protein that appears to be predominantly regulated by p38 in cells. Indeed, MK-2 was the first substrate of p38α to be identified. For example, in vitro phosphorylation of MK-2 by p38α activates MK-2. The substrates that MK-2 acts upon, in turn, include heat shock protein 27, lymphocyte-specific protein 1 (LAP1), cAMP response element-binding protein (CREB), ATF1, serum response factor (SRF), and tyrosine hydroxylase. The substrate of MK-2 that has been best characterized is small heat shock protein 27 (hsp27).

The role of the p38 pathway in inflammatory-related diseases has been studied in several animal models. The pyridinyl imidazole compound SB203580 has been shown to be a specific inhibitor of p38 in vivo, and also has been shown to inhibit activation of MK-2, (See, Rouse, J., et al, Cell, 78:1027-1037 (1994); Cuenda, A., et al, Biochem. J., 333:11-15 (1998)), as well as a MAP kinase homologue termed reactivating kinase (RK). (See, Cuenda, A., et al., FEBS Lett., 364(2):229-233 (1995)). Inhibition of p38 by SB203580 can reduce mortality in a murine model of endotoxin-induced shock and inhibit the development of mouse collagen-induced arthritis and rat adjuvant arthritis. See, e.g., Badger, A. M., et al., J. Pharmacol Exp. Ther., 279:1453-1461 (1996). Another p38 inhibitor that has been utilized in an animal model that is believed to be more potent than SB203580 in its inhibitory effect on p38 is SB 220025. A recent animal study has demonstrated that SB 220025 caused a significant dose-dependent decrease in vascular density of granulomas in laboratory rats. (See, Jackson, J. R., et al, J. Pharmacol. Exp. Ther., 284:687-692 (1998)). The results of these animal studies indicated that p38, or the components of the p38 pathway, can be useful therapeutic targets for the prevention or treatment of inflammatory disease.

Due to its integral role in the p38 signaling pathway, MK-2 has been used as a monitor for measuring the level of activation in the pathway. Because of its downstream location in the pathway, relative to p38, MK-2 has been measured as a more convenient, albeit indirect, method of assessing p38 activation. However, so far, research efforts exploring therapeutic strategies associated with the modulation of this pathway have focused mainly on the inhibition of p38 kinase.

Several compounds that inhibit the activity of p38 kinase have been described in U.S. Pat. Nos. 6,046,208, 6,251,914, and 6,335,340. These compounds have been suggested to be useful for the treatment of CSBP/RK/p38 kinase mediated disease. Commercial efforts to apply p38 inhibitors have centered around two p38 inhibitors, the pyridinylimidazole inhibitor SKF 86002, and the 2,4,5 triaryl imidazole inhibitor SB203580. See, Lee, J. C., et al, Immunopharmacology 47, 185-192 (2000). Compounds possessing a similar structure have also been investigated as potential p38 inhibitors. Indeed, p38 MSP kinase's role in various disease states has been elucidated through the use of inhibitors.

Kotlyarov, A. et al, in Nat. Cell Biol., 1(2):94-97 (1999) introduced a targeted mutation into a mouse MK-2 gene, resulting in MK-2-deficient mice. It was shown that mice lacking MK-2 possessed increased stress resistance and survived LPS-induced endotoxic shock better than MK-2⁺ mice. The authors concluded that MK-2 was an essential component in the inflammatory response that regulates biosynthesis of TNFα at a post-transcriptional level. More recently, Lehner, M. D., et al, in J. Immunol., 168(9):4667-4673 (2002), reported that MK-2-deficient mice showed increased susceptibility to Listeria monocytogenes infection, and concluded that MK-2 had an essential role in host defense against intracellular bacteria, probably via regulation of TNF and IFN-gamma production required for activation of antibacterial effector mechanisms.

The location of MK-2 in the p38 signaling pathway at a point that is downstream of p38 offers the potential that MK-2 could act as a focal point for modulating the pathway without affecting as many substrates as would the regulation of an enzyme further upstream in the signaling cascade—such as p38 MAP kinase.

Accordingly, it would be useful to provide compounds and methods that could serve to modulate the activity of MK-2—in particular, to act as inhibitors of MK-2 activity. Such compounds and methods would be useful for the provision of benefits similar to p38 MAP kinase inhibitors, which benefits include the prevention and treatment of diseases and disorders that are mediated by TNFα. It would be even more useful to provide MK-2 inhibitors having improved potency and reduced undesirable side effects, relative to p38 inhibitors.

### SUMMARY OF THE INVENTION

Briefly, therefore the present invention is directed to a novel aminocyanopyridine compound, or a pharmaceutically acceptable salt thereof, the compound having the structure:

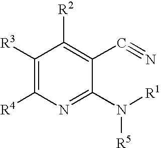

wherein:

R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-(C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;

R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N-C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy,

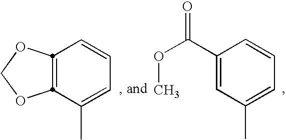

with the proviso that when R² is aryl, it is not substituted with nitro;

R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di- C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except that when R² is heteroaryl, R³ is other than cyano, and

where the R² and R³ groups are such that they optionally join to form a ring system selected from:

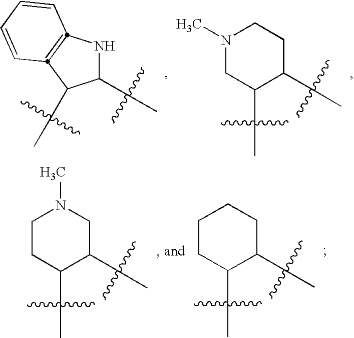

R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups are optionally substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy

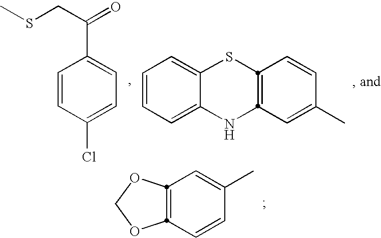

wherein the R³ and R⁴ groups are such that they optionally join to form a ring system selected from:

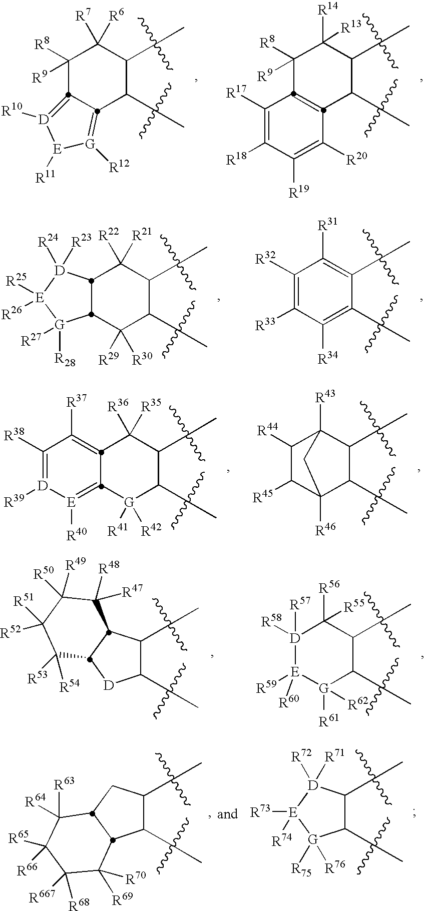

D, E and G are each independently selected from carbon, oxygen, sulfur, and nitrogen;

R⁵ is selected from the group consisting of —H, and C₁-C₅ alkyl, except that at least one of R¹, R², R³, R⁴, and R⁵ is other than hydrogen; and

wherein the R¹ and R⁵ groups optionally join to form a piperidyl ring or a oxaxinyl ring;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl-ethyl ester, pyridyl C₁-C₄ alkyl, pyridyl C₁-C₄ alkoxy, —COO—CH₂—CH₃, with the proviso that when E is —N—, R³⁸ is not cyano, and that when G is —N—, R³⁶ is —H; and

wherein R³⁸ and R³⁹ are such that they optionally join to form a ring system of the type selected from:

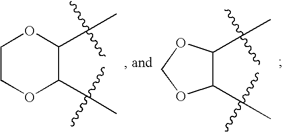

with the proviso that when R¹, R³ and R⁵ are hydrogen:

R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};

where Z₁ and Z₂ are each independently selected from the group consisting of hydrogen, alkoxycarbonyl, alkyl, alkylcarbonyl, benzyl, benzyloxycarbonyl, and formyl;

R^{A} is selected from the group consisting of aryl and arylalkyl;

R^{B} is selected from the group consisting of aryl, arylalkoxy, arylalkyl, aryloxy, heterocycle, and heterocyclealkyl; and

R⁴ is other than alkenyl, alkoxyalkynyl, alkyl, alkynyl, cycloalkyl, aryl, arylalkyl, heterocycle, heterocyclealkyl, or —R_{C}R_{D}R_{E};

where R_{C} is selected from the group consisting of aryl, arylalkyl, heterocycle and heterocyclealkyl;

R_{D} is selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl; and

R_{E} is absent or selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl.

The invention is also directed to a novel pharmaceutical composition comprising a pharmaceutically acceptable carrier and an aminocyanopyridine compound, or a pharmaceutically acceptable salt thereof, the compound having the structure:

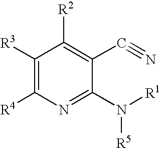

wherein:

R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-( C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;

R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy,

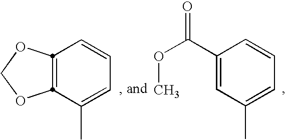

with the proviso that when R² is aryl, it is not substituted with nitro;

R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di- C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except when R² is heteroaryl, R³ is other than cyano; and

where the R² and R³ groups are such that they optionally join to form a ring system selected from:

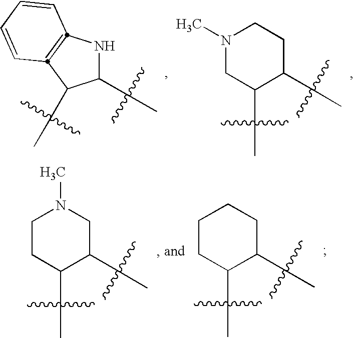

R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups are optionally substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy

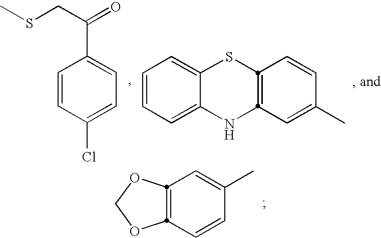

wherein the R³ and R⁴ groups are such that they optionally join to form a ring system selected from:

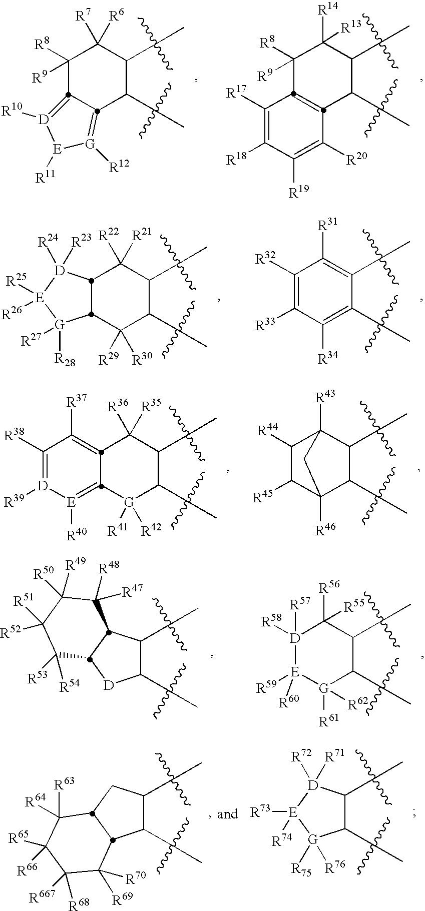

D, E and G are each independently selected from carbon, oxygen, sulfur, and nitrogen;

R⁵ is selected from the group consisting of —H, and C₁-C₅ alkyl, provided that at least one of R¹, R², R³, R⁴, and R⁵ is other than hydrogen; and

wherein the R¹ and R⁵ groups optionally join to form a piperidyl ring or a oxaxinyl ring;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl - ethyl ester, pyridyl C₁-C₄ alkyl, pyridyl C₁-C₄ alkoxy, —COO—CH₂—CH₃, with the proviso that when E is —N—, R³⁸ is other than cyano, and that when G is —N—, R³⁶ is —H; and

wherein R³⁸ and R³⁹ are such that they optionally join to form a ring system of the type selected from:

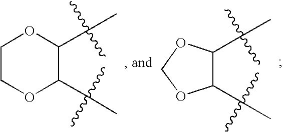

with the proviso that when R¹, R³ and R⁵ are hydrogen:

R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};

where Z₁ and Z₂ are each independently selected from the group consisting of hydrogen, alkoxycarbonyl, alkyl, alkylcarbonyl, benzyl, benzyloxycarbonyl, and formyl;

R^{A} is selected from the group consisting of aryl and arylalkyl;

R^{B} is selected from the group consisting of aryl, arylalkoxy, arylalkyl, aryloxy, heterocycle, and heterocyclealkyl; and

R⁴ is other than alkenyl, alkoxyalkynyl, alkyl, alkynyl, cycloalkyl, aryl, arylalkyl, heterocycle, heterocyclealkyl, or —R_{C}R_{D}R_{E};

where R_{C} is selected from the group consisting of aryl, arylalkyl, heterocycle and heterocyclealkyl;

R_{D} is selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl; and

R_{E} is absent or selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl.

The present invention is also directed to a novel kit for the purpose of treating a TNFα mediated disease or disorder, the kit comprising a dosage form comprising an aminocyanopyridine compound, or a pharmaceutically acceptable salt thereof, the compound having the structure:

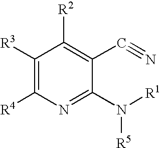

wherein:

R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-(C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;

R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy,

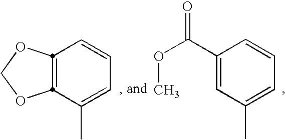

with the proviso that when R² is aryl, it is not substituted with nitro;

R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di- C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except when R² is heteroaryl, R³ is other than cyano, and

where the R² and R³ groups are such that they optionally join to form a ring system selected from:

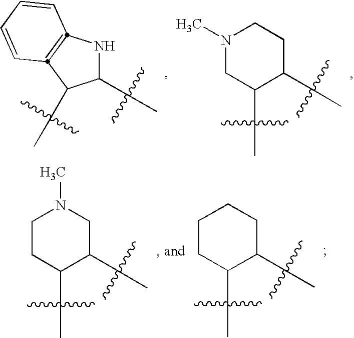

R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups are optionally substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy

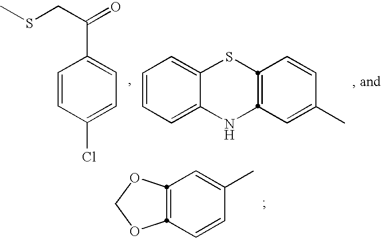

wherein the R³ and R⁴ groups are such that they optionally join to form a ring system selected from:

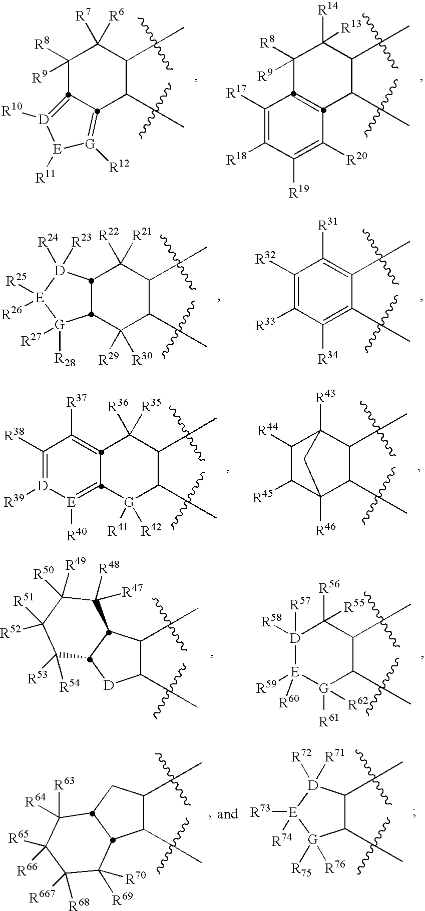

D, E and G are each independently selected from carbon, oxygen, sulfur, and nitrogen;

R⁵ is selected from the group consisting of —H, and C₁-C₅ alkyl, provided that at least one of R¹, R², R³, R⁴ and R⁵ is other than hydrogen; and

wherein the R¹ and R⁵ groups optionally join to form a piperidyl ring or a oxaxinyl ring;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl-ethyl ester, pyridyl C₁-C₄ alkyl, pyridyl C₁-C₄ alkoxy, —COO—CH₂—CH₃, with the proviso that when E is —N—, R³⁸ is other than cyano, and that when G is —N—, R³⁶ is —H; and

wherein R³⁸ and R³⁹ are such that they optionally join to form a ring system of the type selected from:

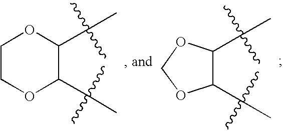

with the proviso that when R¹, R³ and R⁵ are hydrogen:

R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};

where Z₁ and Z₂ are each independently selected from the group consisting of hydrogen, alkoxycarbonyl, alkyl, alkylcarbonyl, benzyl, benzyloxycarbonyl, and formyl;

R^{A} is selected from the group consisting of aryl and arylalkyl;

R^{B} is selected from the group consisting of aryl, arylalkoxy, arylalkyl, aryloxy, heterocycle, and heterocyclealkyl; and

R⁴ is other than alkenyl, alkoxyalkynyl, alkyl, alkynyl, cycloalkyl, aryl, arylalkyl, heterocycle, heterocyclealkyl, or —R_{C}R_{D}R_{E};

where R_{C} is selected from the group consisting of aryl, arylalkyl, heterocycle and heterocyclealkyl;

R_{D} is selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl; and

R_{E} is absent or selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl.

Among the several advantages found to be achieved by the present invention, therefore, may be noted the provision of a compound that could serve to modulate the activity of MK-2—in particular, to inhibit MK-2 activity, and the provision of a compound that could be useful for the prevention and treatment of diseases and disorders that are mediated by TNFα.

### DETAILED DESCRIPTION OF THE PREFERRED EMBODIMENTS

In accordance with the present invention, it has been discovered that certain aminocyanopyridine compounds can inhibit the activity of MAPKAP kinase-2. Many of these compounds exhibit their inhibitory effect at low concentrations—having in vitro MK-2 inhibition IC₅₀ values of under 10 μM, and with some having IC₅₀ values of under about 5 μM, and even as low as about 1.2 μM. Accordingly, these compounds can be potent and effective drugs for use in methods to prevent or treat diseases and disorders that are mediated by TNFα. For example, they can be used for the prevention or treatment of arthritis.

Aminocyanopyridine compounds that are useful in the present method include those having the structure shown in formula I:

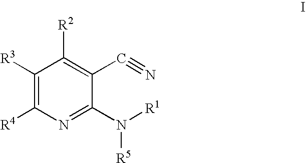

wherein:

R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-(C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;

R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy,

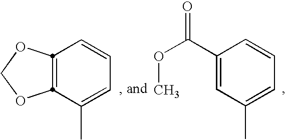

with the proviso that when R² is aryl, it is not substituted with nitro;

R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di- C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except when R² is heteroaryl, R³ is other than cyano; and

where the R² and R³ groups are such that they optionally join to form a ring system selected from:

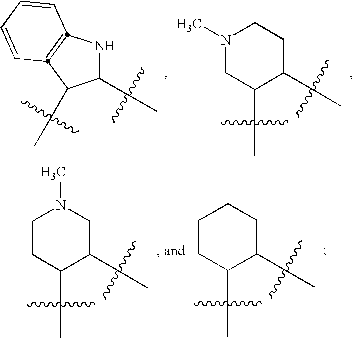

As shown above, ring substituent groups that join to form additional ring structures adjacent the substituted ring can be described with reference to chemical formulas that show wavy lines to indicate that a partial molecule is shown. In these formulas, the wavy lines cut through the ring to which the substituents are joined (in this case, the pyridine ring of formula I), rather than across the bond joining the substituent group to the ring. Accordingly, the partial ring that is shown is the ring to which the substituent groups are shown as being bonded in the general formula.

R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups are optionally substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy

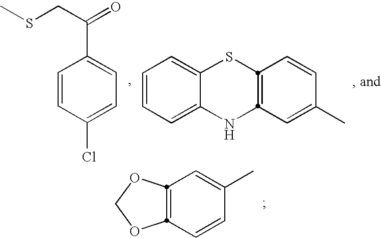

wherein the R³ and R⁴ groups are such that they optionally join to form a ring system selected from:

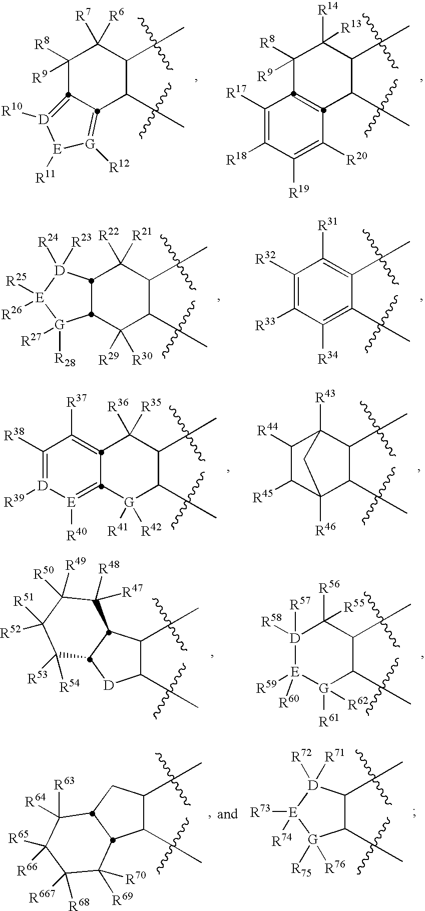

D, E and G are each independently selected from carbon, oxygen, sulfur, and nitrogen;

R⁵is selected from the group consisting of —H, and C₁-C₅ alkyl, provided that at least one of R¹, R², R³, R⁴, and R⁵ is other than hydrogen; and

wherein the R¹ and R⁵ groups optionally join to form a piperidyl ring or a oxaxinyl ring;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl - ethyl ester, pyridyl C₁-C₄ alkyl, pyridyl C₁-C₄ alkoxy,—COO—CH₂—CH₃, with the proviso that when E is —N—, R³⁸ is other than cyano, and that when G is —N—, R³⁶ is —H; and

wherein R³⁸ and R³⁹ are such that they optionally join to form a ring system of the type selected from:

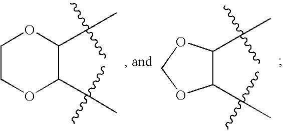

with the proviso that when R¹, R³ and R⁵ are hydrogen:

R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};

where Z₁ and Z₂ are each independently selected from the group consisting of hydrogen, alkoxycarbonyl, alkyl, alkylcarbonyl, benzyl, benzyloxycarbonyl, and formyl;

R^{A} is selected from the group consisting of aryl and arylalkyl;

R^{B} is selected from the group consisting of aryl, arylalkoxy, arylalkyl, aryloxy, heterocycle, and heterocyclealkyl; and

R⁴ is other than alkenyl, alkoxyalkynyl, alkyl, alkynyl, cycloalkyl, aryl, arylalkyl, heterocycle, heterocyclealkyl, or —R_{C}R_{D}R_{E};

where R_{C} is selected from the group consisting of aryl, arylalkyl, heterocycle and heterocyclealkyl;

R_{D} is selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl; and

R_{E} is absent or selected from the group consisting of aryl, arylalkoxy, arylalkoxyimino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxyimino, heterocycleoxyiminoalkyl, and heterocyclesulfonyl.

As used herein, the term “alkyl”, alone or in combination, means an acyclic alkyl-radical, linear or branched, which, unless otherwise noted, preferably contains from 1 to about 10 carbon atoms and more preferably contains from 1 to about 6 carbon atoms. “Alkyl” also encompasses cyclic alkyl radicals containing from 3 to about 7 carbon atoms, preferably from 3 to 5 carbon atoms. The alkyl radicals can be optionally substituted with groups as defined below. Examples of such alkyl radicals include methyl, ethyl, chloroethyl, hydroxyethyl, n-propyl, isopropyl, n-butyl, cyanobutyl, isobutyl, sec-butyl, tert-butyl, pentyl, aminopentyl, iso-amyl, hexyl, octyl, and the like.

The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like.

The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “alkoxy” includes linear or branched oxy-containing radicals, each of which has, unless otherwise noted, alkyl portions of 1 to about 6 carbon atoms, preferably 1 to about 4 carbon atoms, such as methoxy, ethoxy, propoxy, isopropoxy, isobutoxy radicals, and the like.

The term “alkoxyalkyl” also embraces alkyl radicals having one or more alkoxy radicals attached to the alkyl radical, that is, to form monoalkoxyalkyl and dialkoxyalkyl radicals. Examples of such radicals include methoxyalkyls, ethoxyalkyls, propoxyalkyls, isopropoxyalkyls, butoxyalkyls, tert-butoxyalkyls, and the like. The “alkoxy” radicals may be further substituted with one or more halo atoms, such as fluoro, chloro, or bromo, to provide “haloalkoxy” radicals. Examples of such radicals includ fluoromethoxy, chloromethoxy, trifluoromethoxy, difluoromethoxy, trifluoroethoxy, fluoroethoxy, tetrafluoroethoxy, pentafluoroethoxy, fluoropropoxy, and the like.

The term “alkylthio” embraces radicals containing a linear or branched alkyl radical, preferably, unless otherwise noted, of from 1 to about 6 carbon atoms, attached to a divalent sulfur atom. An example of “lower alkylthio”, is methylthio (CH₃—S—).

The term “alkylthioalkyl” embraces alkylthio radicals, attached to an alkyl group. An example of such radicals is methylthiomethyl.

The term “halo” means radicals comprising halogens, such as fluorine, chlorine, bromine, or iodine.

The term “heterocyclyl” means a saturated or unsaturated mono- or multi-ring carbocycle wherein one or more carbon atoms is replaced by N, S, P, or O. This includes, for example, structures such as:

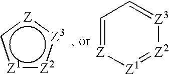

where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others.

The term “heteroaryl” means a fully unsaturated heterocycle, which can include, but is not limited to, furyl, thenyl, pyrryl, imidazolyl, pyrazolyl, pyridyl, thiazolyl, quinolinyl, isoquinolinyl, benzothienyl, and indolyl.

In either, “heterocyclyl” or “heteroaryl”, the point of attachment to the molecule of interest can be at the heteroatom or elsewhere within the ring.

The term “cycloalkyl” means a mono- or multi-ringed carbocycle wherein each ring contains three to about seven carbon atoms, preferably three to about six carbon atoms, and more preferably three to about five carbon atoms. Examples include radicals, such as cyclopropyl, cyclobutyl, cyclopentyl, cyclohexyl, cycloalkenyl, and cycloheptyl. The term “cycloalkyl” additionally encompasses spiro systems wherein the cycloalkyl ring has a carbon ring atom in common with the seven-membered heterocyclic ring of the benzothiepine.

The term “oxo” means a doubly-bonded oxygen.

The term “aryl” means a fully unsaturated mono- or multi-ring carbocycle, including, but not limited to, substituted or unsubstituted phenyl, naphthyl, or anthracenyl.

The present aminocyanopyridine compounds inhibit the activity of the MK-2 enzyme. When it is said that a subject compound inhibits MK-2, it is meant that the MK-2 enzymatic activity is lower in the presence of the compound than it is under the same conditions in the absence of such compound. One method of expressing the potency of a compound as an MK-2 inhibitor is to measure the “IC₅₀” value of the compound. The IC₅₀ value of an MK-2 inhibitor is the concentration of the compound that is required to decrease the MK-2 enzymatic activity by one-half. Accordingly, a compound having a lower IC₅₀ value is considered to be a more potent inhibitor than a compound having a higher IC₅₀ value. As used herein, aminocyanopyridine compounds that inhibit MK-2 can be referred to as aminocyanopyridine MK-2 inhibitors, or aminocyanopyridine MK-2 inhibiting compounds or MK-2 inhibiting agents.

Examples of aminocyanopyridine compounds that are suitable for use as MK-2 inhibitors in the present invention are shown in Table I.

### Table I: Aminocyanopyridine MK-2 Inhibitors

| | | | MK-2 |
|---|---|---|---|
| | | | Avg. |
| | | | IC50 |
| No. | Structure^{a} | Compound Name(s)^{b} | (μM) |
| 1 | 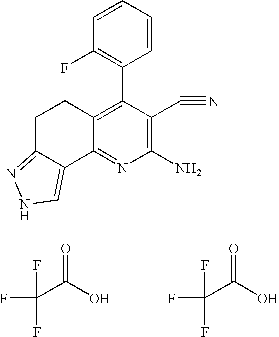 | 2-amino-4-(2-fluorophenyl)-6,8- dihydro-5H-pyrazolo[3,4-h]quinoline-3- carbonitrile bis(trifluoroacetate) | 1.22 |
| 2 | 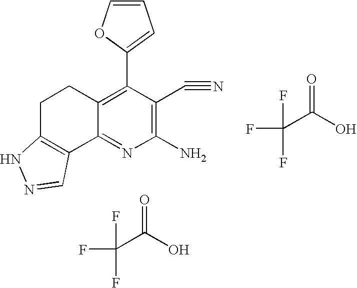 | 2-amino-4-(2-furyl)-6,7-dihydro-5H- pyrazolo[3,4-h]quinoline-3-carbonitrile bis(trifluoroacetate) | 1.36 |
| 3 | 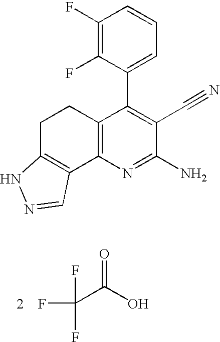 | 2-amino-4-(2,3-difluorophenyl)-6,7- dihydro-5H-pyrazolo[3,4-h]quinoline-3- carbonitrile bis(trifluoroacetate) | 195 |
| 4 | 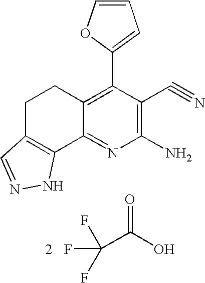 | 8-amino-6-(2-furyl)-4,5-dihydro-1H- pyrazolo[4,3-h]quinoline-7-carbonitrile | 1.96 |
| 5 | 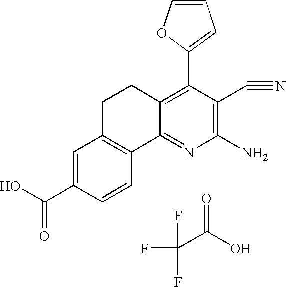 | 2-amino-3-cyano-4-(2-furyl)-5,6- dihydrobenzo[h]quinoline-8-carboxylic acid trifluoroacetate | 2.35 |
| 6 | 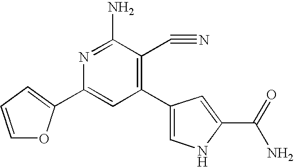 | 4-[2-amino-3-cyano-6-(2-furyl)pyridin- 4-yl]-1H-pyrrole-2-carboxamide | 2.41 |
| 7 | 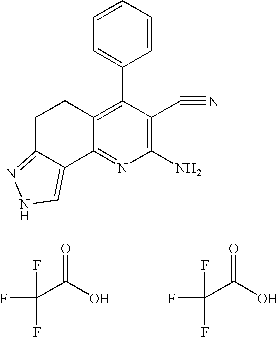 | 2-amino-4-phenyl-6,8-dihydro-5H- pyrazolo[3,4-h]quinoline-3-carbonitrile bis(trifluoroacetate) | 2.73 |
| 8 | 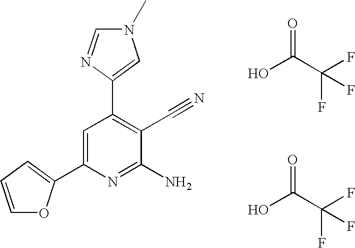 | 2-amlno-6-(2-furyl)-4-(1-methyl-1H- imidazol-4-yl)nicotinonitrile bis(trifluoroacetate) | 2.88 |
| 9 | 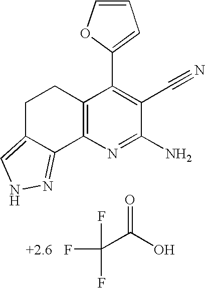 | 8-amino-6-(2-furyl)-4,5-dihydro-1H- pyrazolo[4,3-h]quinoline-7-carbonitrile trifluoroacetate | 3.23 |
| 10 | 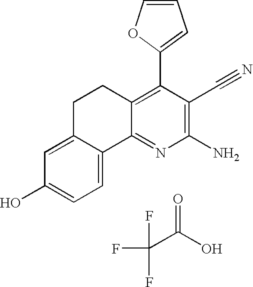 | 2-amino-4-(2-furyl)-8-hydroxy-5,6- dihydrobenzo[h]quinoline-3- carbonitrile trifluoroacetate | 3.48 |
| 11 | 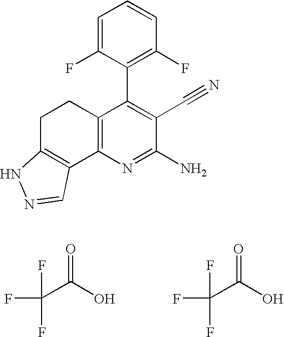 | 2-amino-4-(2,6-difluorophenyl)-6,7- dihydro-5H-pyrazolo[3,4-h]quinoline-3- carbonitrile bis(trifluoroacetate) | |
| 12 | 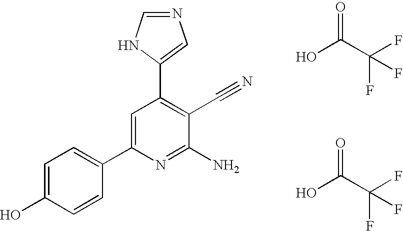 | 2-amino-6-(4-hydroxyphenyl)-4-(1H- imidazol-5-yl)nicotinonitrile trifluoroacetate | 3.62 |
| 13 | 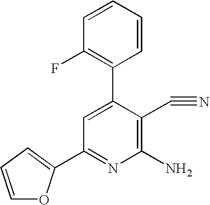 | 2-amino-4-(2-fluorophenyl)-6-(2- furyl)nicotinonitrile | 4.06 |
| 14 | 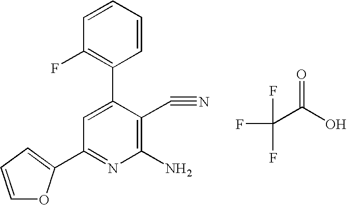 | 2-amino-4-(2-fluorophenyl)-6-(2- furyl)nicotinonitrile trifluoroacetate | 4.41 |
| 15 | 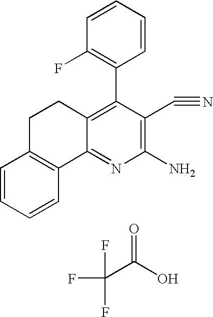 | 2-amino-4-(2-fluorophenyl)-5,6- dihydrobenzo[h]quinoline-3- carbonitrile trifluoroacetate | 4.47 |
| 16 | 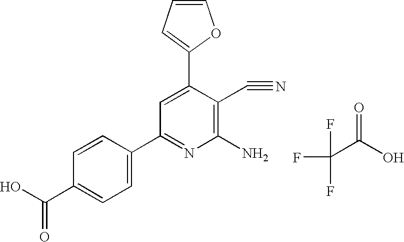 | 4-[6-amino-5-cyano-4-(2-furyl)pyridin- 2-yl]benzoic acid trifluoroacetate | 4.63 |
| 17 | 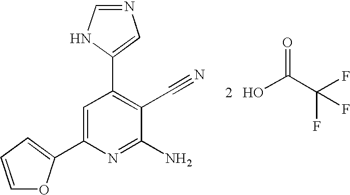 | 2-amino-6-(2-furyl)-4-(1H-imidazol-5- yl)nicotinonitrile trifluoroacetate | 4.94 |
| 18 | 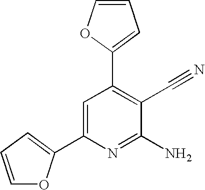 | 2-amino-4-(2-furyl)-6-(1H-pyrazol-3- yl)nicotinonitrile | 5.46 |
| 19 | 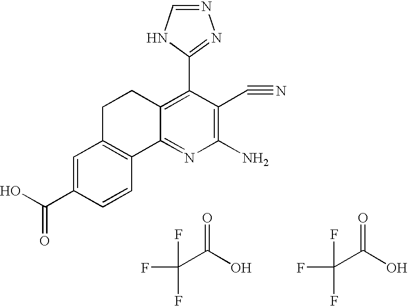 | 2-amino-3-cyano-4-(4H-1,2,4-triazol-3- yl)-5,6-dihydrobenzo[h]quinoline-8- carboxylic acid bis(trifluoroacetate) | 5.74 |
| 20 | 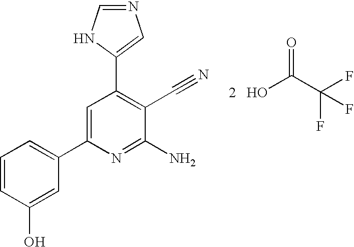 | 2-amino-6-(3-hydroxyphenyl)-4-(1H- imidazol-5-yl)nicotinonitrile trifluoroacetate | 5.81 |
| 21 | 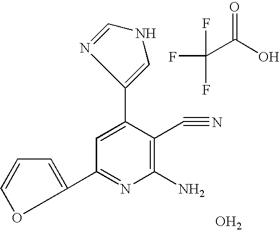 | 2-amino-6-(2-furyl)-4-(1H-imidazol-4- yl)nicotinonitrile trifluoroacetate hydrate | 5.95 |
| 22 | 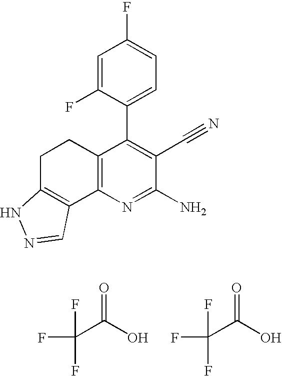 | 2-amino-4-(2,4-difluorophenyl)-6,7- dihydro-5H-pyrazolo[3,4-h]quinoline-3- carbonitrile bis(trifluoroacetate) | |
| 23 | 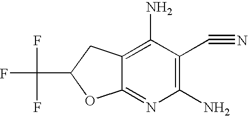 | 4,6-diamino-2-(trifluoromethyl)-2,3- dihydrofuro[2,3-b]pyridine-5- carbonitrile or 6N009 | 6.14 |
| 24 | 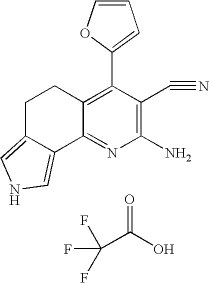 | 2-amino-4-(2-furyl)-6,8-dihydro-5H- pyrrolo[3,4-h]quinoline-3-carbonitrile trifluoroacetate | 6.2 |
| 25 | 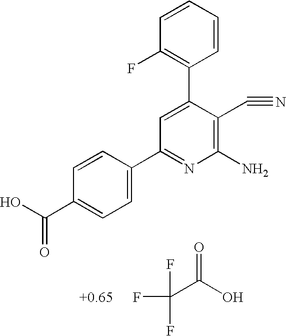 | 4-[6-amino-5-cyano-4-(2- fluorophenyl)pyridin-2-yl]benzoic acid trifluoroacetate | 6.4 |
| 26 | 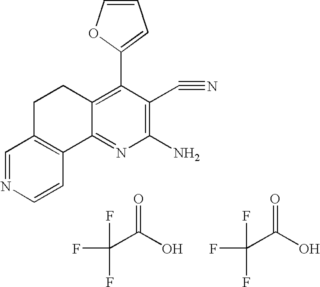 | 2-amino-4-(2-furyl)-5,6-dihydro-1,8- phenanthroline-3-carbonitrile bis(trifluoroacetate) | 6.48 |
| 27 | 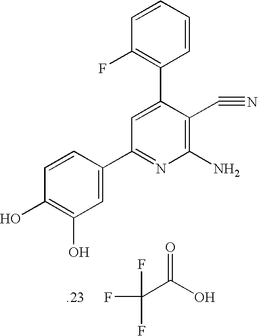 | 2-amino-6-(3,4-dihydroxyphenyl)-4-(2- fluorophenyl)nicotinonitrile trifluoroacetate | 7.54 |
| 28 | 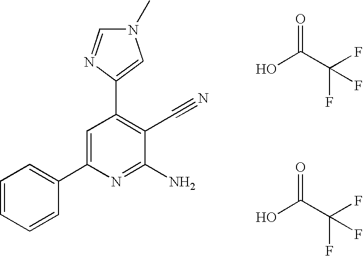 | 2-amino-4-(1-methyl-1H-imidazol-4-yl) 6-phenylnicotinonitrile bis(trifluoroacetate) | 7.63 |
| 29 | 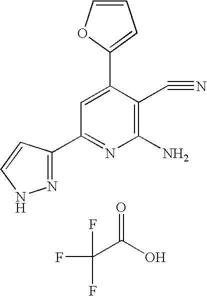 | 2-amino-4-(2-furyl)-6-(1H-pyrazol-3- yl)nicotinonitrile trifluoroacetate | 7.72 |
| 30 | 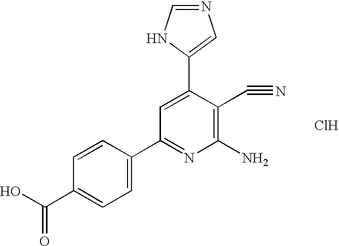 | 4-[6-amino-5-cyano-4-(1H-imidazol-5- yl)pyridin-2-yl]benzoic acid hydrochloride | 8.37 |
| 31 | 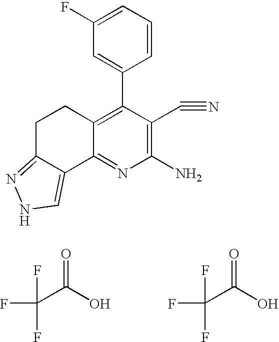 | 2-amino-4-(3-fluorophenyl)-6,8- dihydro-5H-pyrazolo[3,4-h]quinoline-3- carbonitrile bis(trifluoroacetate) | 8.5 |
| 32 | 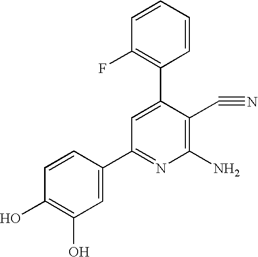 | 2-amino-6-(3,4-dihydroxyphenyl)-4-(2- fluorophenyl)nicotinonitrile | 9.2 |
| 33 | 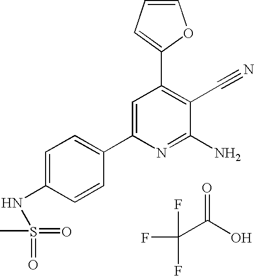 | N-{4-[6-amino-5-cyano-4-(2- furyl)pyridin-2- yl]phenyl}methanesulfonamide trifluoroacetate | 9.27 |
| 34 | 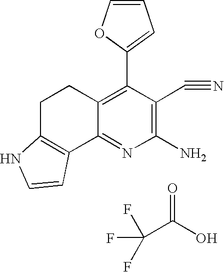 | 2-amino-4-(2-furyl)-6,7-dihydro-5H- pyrrolo[2,3-h]quinoline-3-carbonitrile trifluoroacetate | 9.4 |
| 35 | 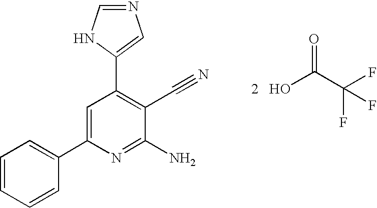 | 2-amino-4-(1H-imidazol-5-yl)-6- phenylnicotinonitrile trifluoroacetate | 9.4 |
| 36 | 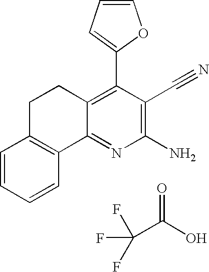 | 2-amino-4-(2-furyl)-5,6- dihydrobenzo[h]quinoline-3- carbonitrile trifluoroacetate | 9.42 |
| 37 | 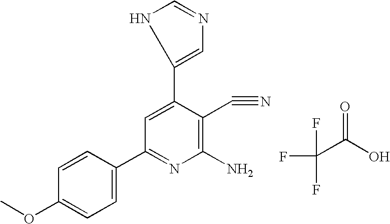 | 2-amino-4-(1H-imidazol-5-yl)-6-(4- methoxyphenyl)nicotinonitrile trifluoroacetate | 9.43 |
| 38 | 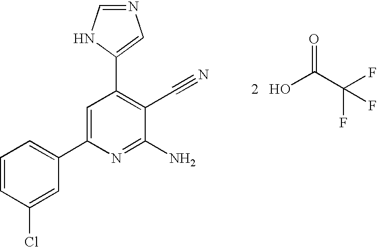 | 2-amino-6-(3-chlorophenyl)-4-(1H- imidazol-5-yl)nicotinonitrile trifluoroacetate | 10 |
| 39 | 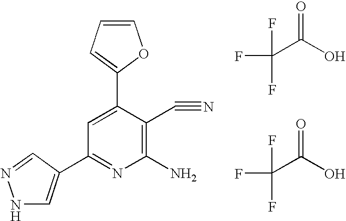 | 2-amino-4-(2-furyl)-6-(1H-pyrazol-4- yl)nicotinonitrile bis(trifluoroacetate) | 11.6 |
| 40 | 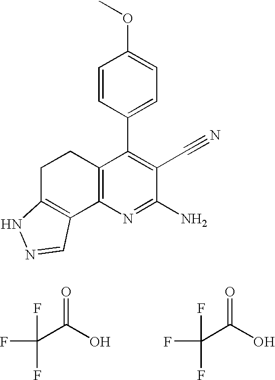 | 2-amino-4-(4-methoxyphenyl)-6,7- dihydro-5H-pyrazolo[3,4-h]quinoline-3- carbonitrile bis(trifluoroacetate) | 12 |
| 41 | 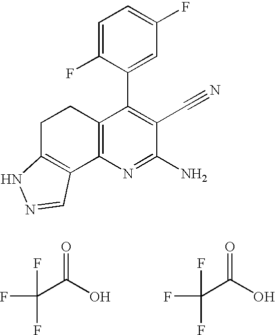 | 2-amino-4-(2,5-difluorophenyl)-6,7- dihydro-5H-pyrazolo[3,4-h]quinoline-3- carbonitrile bis(trifluoroacetate) | 12.8 |
| 42 | 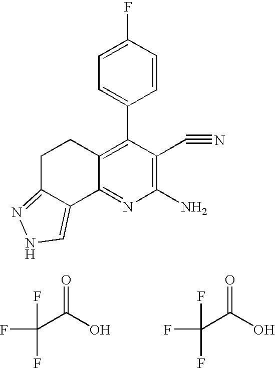 | 2-amino-4-(4-fluorophenyl)-6,8- dihydro-5H-pyrazolo[3,4-h]quinoline-3- carbonitrile bis(trifluoroacetate) | 12.9 |
| 43 | 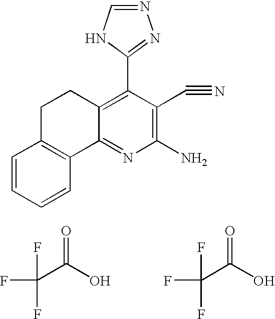 | 2-amino-4-(4H-1,2,4-triazol-3-yl)-5,6- dihydrobenzo[h]quinoline-3- carbonitrile bis(trifluoroacetate) | 13.1 |
| 44 | 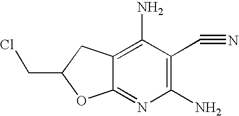 | 4,6-diamino-2-(chloromethyl)-2,3- dihydrofuro[2,3-b]pyridine-5- carbonitrile | 13.4 |
| 45 | 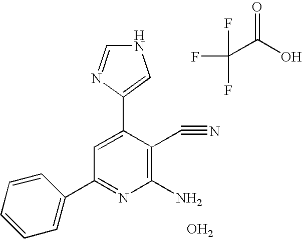 | 2-amino-4-(1H-imidazol-4-yl)-6- phenylnicotinonitrile trifluoroacetate hydrate | 14.2 |
| 46 | 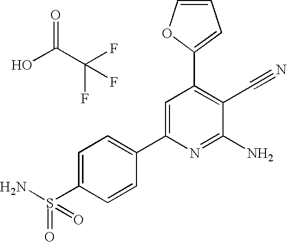 | 4-[6-amino-5-cyano-4-(2-furyl)pyridin- 2-yl]benzenesulfonamide trifluoroacetate | 16.1 |
| 47 | 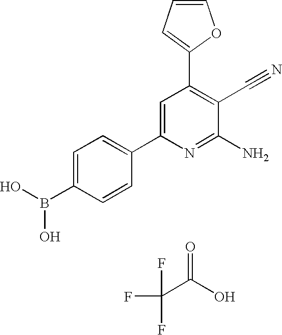 | 4-[6-amino-5-cyano-4-(2-furyl)pyridin- 2-yl]phenylboronic acid trifluoroacetate | 16.7 |
| 48 | 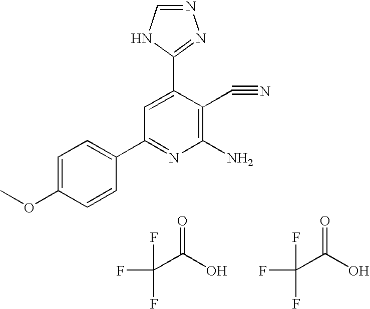 | 2-amino-6-(4-methoxyphenyl)-4-(4H- 1,2,4-triazoi-3-yl)nicotinonitrile bis(trifluoroacetate) | 17.3 |
| 49 | 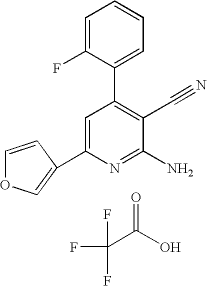 | 2-amino-4-(2-fluorophenyl)-6-(3- furyl)nicotinonitrile trifluoroacetate | 17.9 |
| 50 | 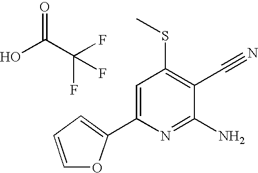 | 2-amino-6-(2-furyl)-4- (methylthio)nicotinonitrile trifluoroacetate | 22.5 |
| 51 | 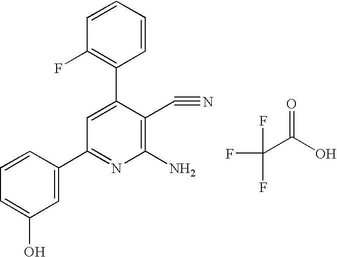 | 2-amino-4-(2-fluorophenyl)-6-(3- hydroxyphenyl)nicotinonitrile trifluoroacetate | 24.2 |
| 52 | 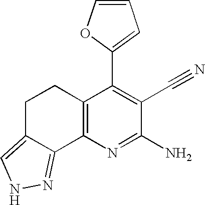 | 8-amino-6-(2-furyl)-4,5-dihydro-2H- pyrazolo[4,3-h]quinoline-7-carbonitrile | 25.3 |
| 53 | 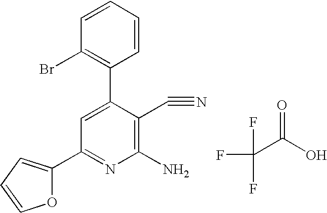 | 2-amino-4-(2-bromophenyl)-6-(2- furyl)nicotinonitrile trifluoroacetate | 26.1 |
| 54 | 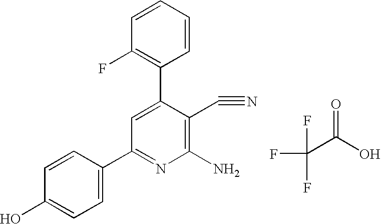 | 2-amino-4-(2-fluorophenyi)-6-(4- hydroxyphenyl)nicotinonitrile trifluoroacetate | 26.8 |
| 55 | 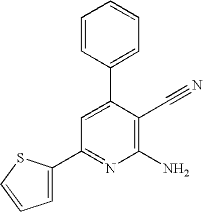 | 2-amino-4-phenyl-6-thien-2- ylnicotinonitrile | 26.9 |
| 56 | 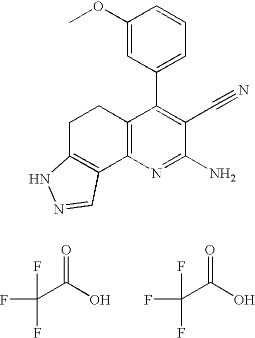 | 2-amino-4-(3-methoxyphenyl)-6,7- dihydro-5H-pyrazolo[3,4-h]quinoline-3- carbonitrile bis(trifluoroacetate) | 27.8 |
| 57 | 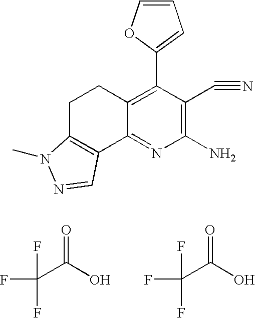 | 2-amino-4-(2-furyl)-7-methyl-6,7- dihydro-5H-pyrazolo[3,4-h]quinoline-3- carbonitrile bis(trifluoroacetate) | 28.3 |
| 58 | 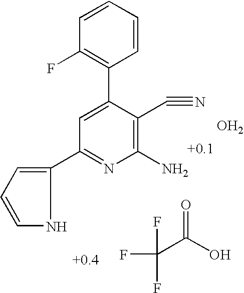 | 2-amino-4-(2-fluorophenyl)-6-(1H- pyrrol-2-yl)nicotinonitrile trifluoroacetate hydrate | 29.3 |
| 59 | 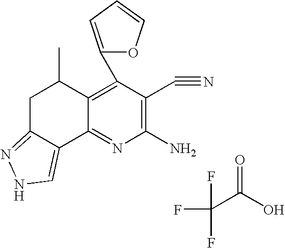 | 2-amino-4-(2-furyl)-5-methyl-6,8- dihydro-5H-pyrazolo[3,4-h]quinoline-3- carbonitrile trifluoroacetate | 31.3 |
| 60 | 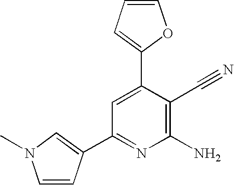 | 2-amino-4-(2-furyl)-6-(1-methyl-1H- pyrrol-3-yl)nicotinonitrile | 32.1 |
| 61 | 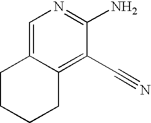 | 3-amino-5,6,7,8- tetrahydroisoquinoline-4-carbonitrile | 33.4 |
| 62 | 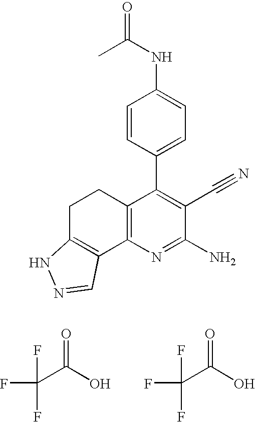 | N-[4-(2-amino-3-cyano-6,7-dihydro- 5H-pyrazolo[3,4-h]quinolin-4- yl)phenyl]acetamide bis(trifluoroacetate) | 35.9 |
| 63 | 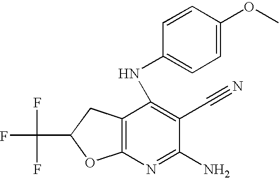 | 6-amino-4-[(4-methoxyphenyl)amino]- 2-(trifluoromethyl)-2,3-dihydrofuro[2,3- b]pyridine-5-carbonitrile | 36.4 |
| 64 | 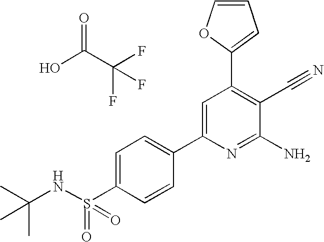 | 4-[6-amino-5-cyano-4-(2-furyl)pyridin- 2-yl]-N-(tert- butyl)benzenesulfonamide trifluoroacetate | 36.4 |
| 65 | 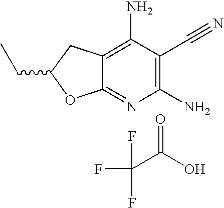 | 4,6-diamino-2-ethyl-2,3- dihydrofuro[2,3-b]pyridine-5- carbonitrile trifluoroacetate | 37.9 |
| 66 | 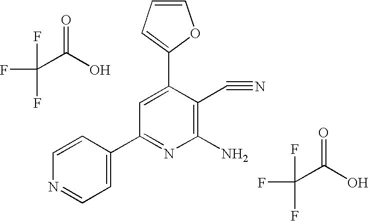 | 6-amino-4-(2-furyl)-2,4′-bipyridine-5- carbonitrile bis(trifluoroacetate) | 39.9 |
| 67 | 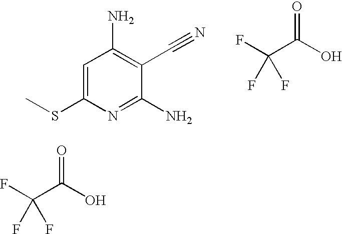 | 2,4-diamino-6- (methylthio)nicotinonitrile bis(trifluoroacetate) | 41.6 |
| 68 | 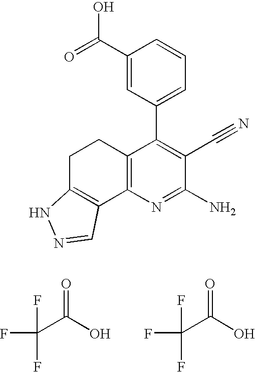 | 3-(2-amino-3-cyano-6,7-dihydro-5H- pyrazolo[3,4-h]quinolin-4-yl)benzoic acid bis(trifluoroacetate) | 41.7 |
| 69 | 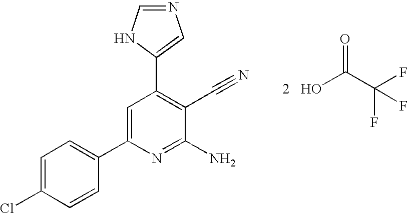 | 2-amino-6-(4-chlorophenyl)-4-(1H- imidazol-5-yl)nicotinonitrile trifluoroacetate | 42.9 |
| 70 | 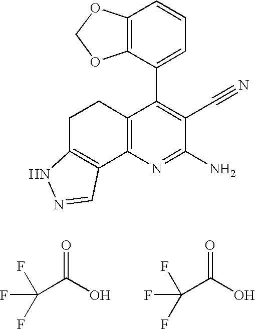 | 2-amino-4-(1,3-benzodioxol-4-yl)-6,7- dihydro-5H-pyrazolo[3,4-h]quinoline-3- carbonitrile bis(trifluoroacetate) | 43.2 |
| 71 | 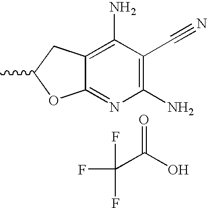 | 4,6-diamino-2-methyl-2,3- dihydrofuro[2,3-b]pyridine-5- carbonitrile trifluoroacetate | 44.1 |
| 72 | 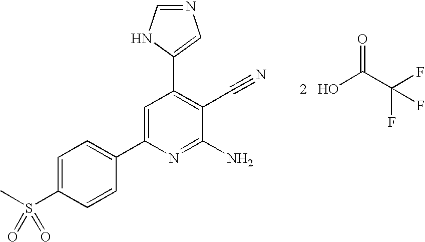 | 2-amino-4-(1H-imidazol-5-yl)-6-[4- (methylsulfonyl)phenyl]nicotinonitrile trifluoroacetate | 45.3 |
| 73 | 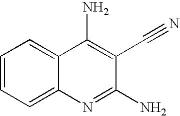 | 2,4-diaminoquinoline-3-carbonitrile | 45.5 |
| 74 | 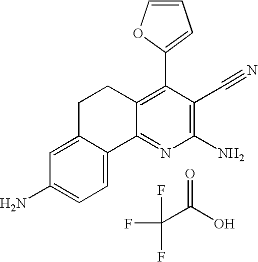 | 2,8-diamino-4-(2-furyl)-5,6- dihydrobenzo[h]quinoline-3- carbonitrile trifluoroacetate | 46.8 |
| 75 | 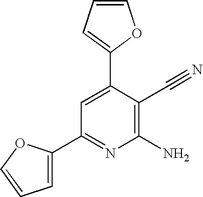 | 2-amino-4,6-di(2-furyl)nicotinonitrile | 47.6 |
| 76 | 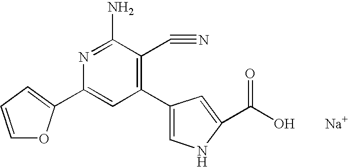 | sodium 4-[2-amino-3-cyano-6-(2- furyl)pyridin-4-yl]-1H-pyrrole-2- carboxylate | 48.7 |
| 77 | 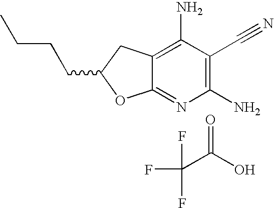 | 4,6-diamino-2-butyl-2,3- dihydrofuro[2,3-b]pyridine-5- carbonitrile trifluoroacetate | 49.1 |
| 78 | 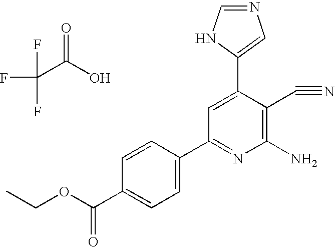 | ethyl 4-[6-amino-5-cyano-4-(1H- imidazol-5-yl)pyridin-2-yl]benzoate trifluoroacetate | 49.1 |
| 79 | 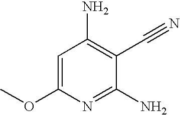 | 2,4-diamino-6-methoxynicotinonitrile | 50.9 |
| 80 | 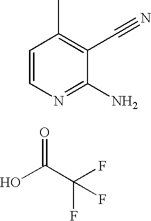 | 2-amino-4-methylnicotinonitrile trifluoroacetate | 51.9 |
| 81 | 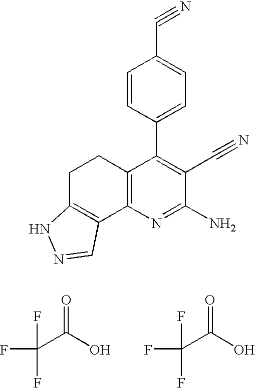 | 2-amino-4-(4-cyanophenyl)-6,7- dihydro-5H-pyrazolo[3,4-h]quinoline-3- carbonitrile bis(trifluoroacetate) | 52.1 |
| 82 | 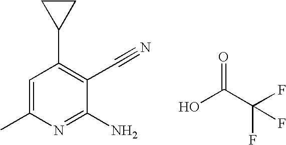 | 2-amino-4-cyclopropyl-6- methylnicotinonitrile trifluoroacetate | 53.7 |
| 83 | 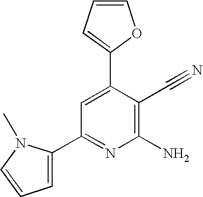 | 2-amino-4-(2-furyl)-6-(1-methyl-1H- pyrrol-2-yl)nicotinonitrile | 54.4 |
| 84 | 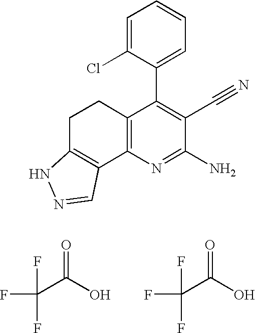 | 2-amino-4-(2-chlorophenyl)-6,7- dihydro-5H-pyrazolo[3,4-h]quinoline-3- carbonitrile bis(trifluoroacetate) | 58.4 |
| 85 | 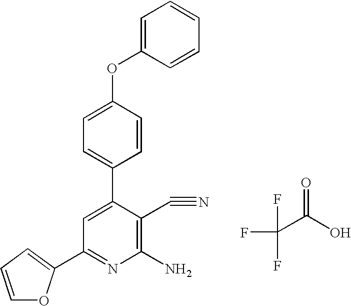 | 2-amino-6-(2-furyl)-4-(4- phenoxyphenyl)nicotinonitrile trifluoroacetate | 59.3 |
| 86 | 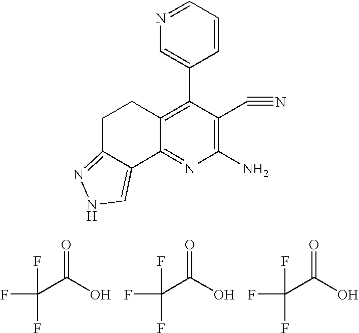 | 2-amino-4-pyridin-3-yl-6,8-dihydro-5H- pyrazolo[3,4-h]quinoline-3-carbonitrile tris(trifluoroacetate) | 62.5 |
| 87 | 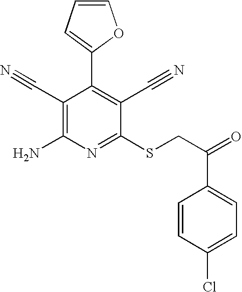 | 2-amino-6-{[2-(4-chlorophenyl)-2- oxoethyl]thio}-4-(2-furyl)pyridine-3,5- dicarbonitrile | 63.3 |
| 88 | 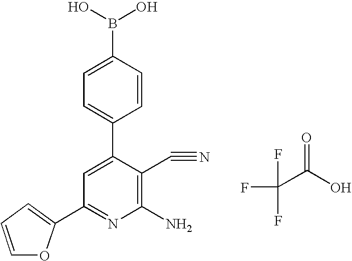 | 4-[2-amino-3-cyano-6-(2-furyl)pyridin- 4-yl]phenylboronic acid trifluoroacetate | 64.6 |
| 89 | 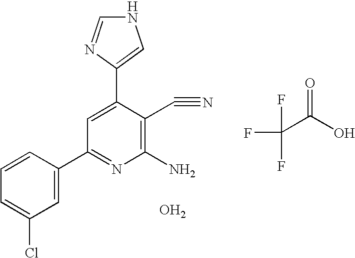 | 2-amino-6-(3-chlorophenyl)-4-(1H- imidazol-4-yl)nicotinonitrile trifluoroacetate hydrate | 64.9 |
| 90 | 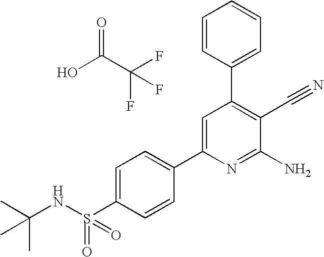 | 4-(6-amino-5-cyano-4-phenylpyridin-2- yl)-N-(tert-butyl)benzenesulfonamide trifluoroacetate | 68 |
| 91 | 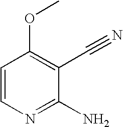 | 2-amino-4-methoxynicotinonitrile | 69.6 |
| 92 | 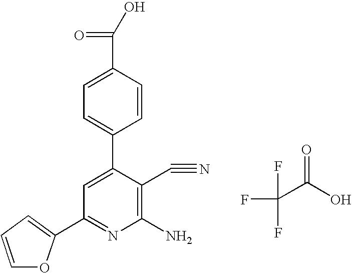 | 4-[2-amino-3-cyano-6-(2-furyl)pyridin- 4-yl]benzoic acid trifluoroacetate | 69.8 |
| 93 | 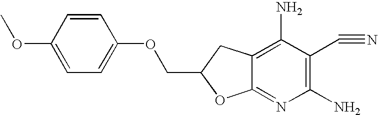 | 4,6-diamino-2-[(4- methoxyphenoxy)methyl]-2,3- dihydrofurol2,3-b]pyridine-5- carbonitrile | 69.8 |
| 94 | 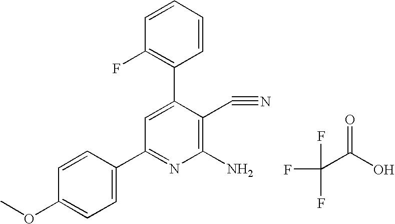 | 2-amino-4-(2-fluorophenyl)-6-(4- methoxyphenyl)nicotinonitrile trifluoroacetate | 70.4 |
| 95 | 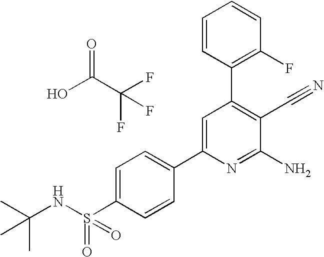 | 4-[6-amino-5-cyano-4-(2- fluorophenyl)pyridin-2-yl]-N-(tert- butyl)benzenesulfonamide trifluoroacetate | 71.5 |
| 96 | 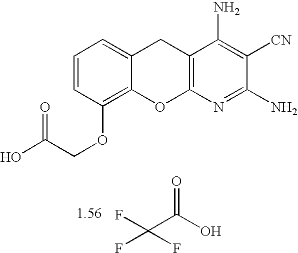 | [(2,4-diamino-3-cyano-5H- chromeno[2,3-b]pyridin-9-yl)oxy]acetic acid trifluoroacetate | 72.2 |
| 97 | 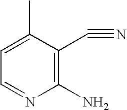 | 3-Pyridinecarbonitrile, 2-Amino-4- Methyl- | 77 |
| 98 | 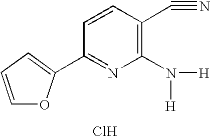 | 2-amino-6-(2-furyl)nicotinonitrile hydrochloride | 77.5 |
| 99 | 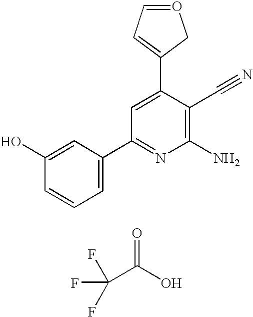 | 2-amino-4-(2-furyl)-6-(3- hydroxyphenyl)nicotinonitrile trifluoroacetate | 77.9 |
| 100 | 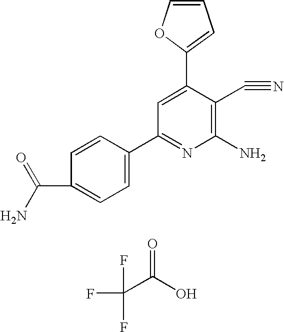 | 4-[6-amino-5-cyano-4-(2-furyl)pyridin- 2-yl]benzamide trifluoroacetate | 78.5 |
| 101 | 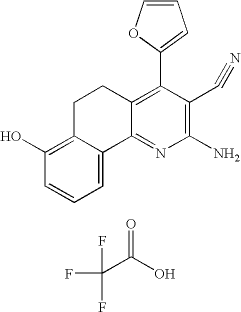 | 2-amino-4-(2-furyl)-7-hydroxy-5,6- dihydrobenzo[h]quinoline-3- carbonitrile trifluoroacetate | 82.6 |
| 102 | 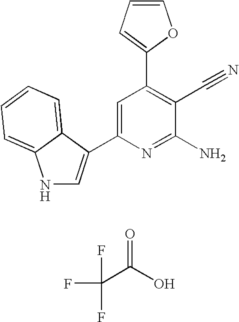 | 2-amino-4-(2-furyl)-6-(1H-indol-3- yl)nicotinonitrile trifluoroacetate | 87.1 |
| 103 | 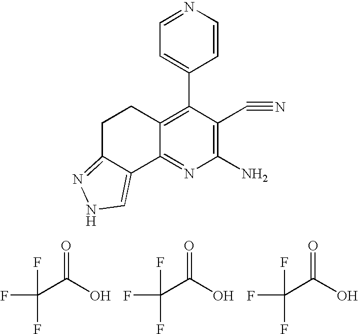 | 2-amino-4-pyridin-4-yl-6,8-dihydro-5H- pyrazolo[3,4-h]quinoline-3-carbonitrile tris(trifluoroacetate) | 94.3 |
| 104 | 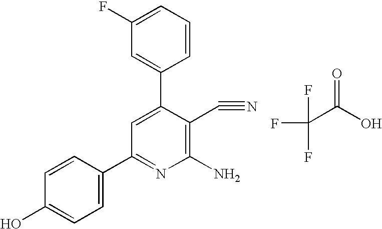 | 2-amino-4-(3-fluorophenyl)-6-(4- hydroxyphenyl)nicotinonitrile trifluoroacetate | 96 |
| 105 | 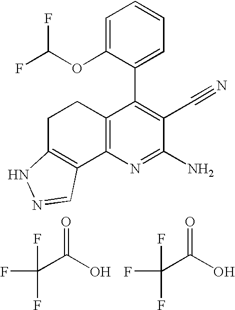 | 2-amino-4-[2-(difluoromethoxy)phenyl]6,7-dihydro-5H-pyrazolo[3,4- h]quinoline-3-carbonitrile bis(trifluoroacetate) | |
| 106 | 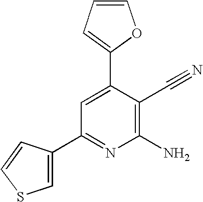 | 2-amino-4-(2-furyl)-6-thien-3- ylnicotinonitrile | 97.3 |
| 107 | 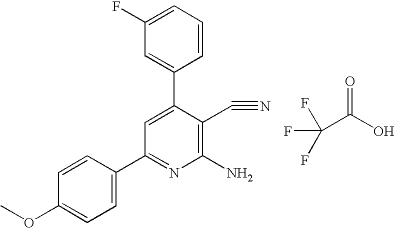 | 2-amino-4-(3-fluorophenyl)-6-(4- methoxyphenyl)nicotinonitrile trifluoroacetate | 97.3 |
| 108 | 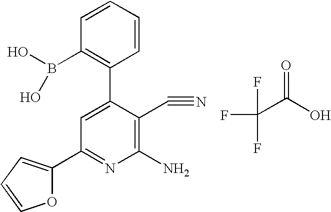 | 2-[2-amino-3-cyano-6-(2-furyl)pyridin- 4-yl]phenylboronic acid trifluoroacetate | 97.3 |
| 109 | 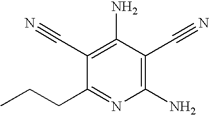 | 2,4-diamino-6-propylpyridine-3,5- dicarbonitrile | 99.8 |
| 110 | 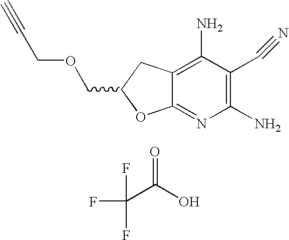 | 4,6-diamino-2-[(prop-2- ynyloxy)methyl]-2,3-dihydrofuro[2,3- b]pyridine-5-carbonitrile trifluoroacetate | 105 |
| 111 | 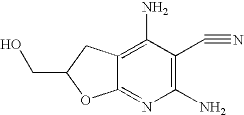 | 4,6-diamino-2-(hydroxymethyl)-2,3- dihydrofuro[2,3-b]pyridine-5- carbonitrile | 106 |
| 112 | 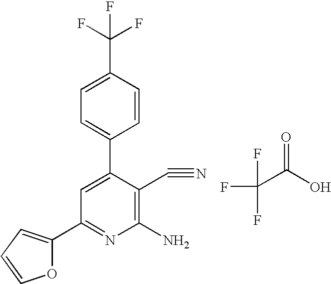 | 2-amino-6-(2-furyl)-4-[4- (trifluoromethyl)phenyl]nicotinonitrile trifluoroacetate | 107 |
| 113 | 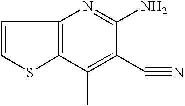 | 5-amino-7-methylthieno[3,2-b]pyridine 6-carbonitrile or GK02302 | 109 |
| 114 | 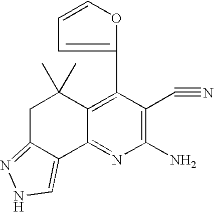 | 2-amino-4-(2-furyl)-5,5-dimethyl-6,8- dihydro-5H-pyrazolo[3,4-h]quinoline-3- carbonitrile | 109 |
| 115 | 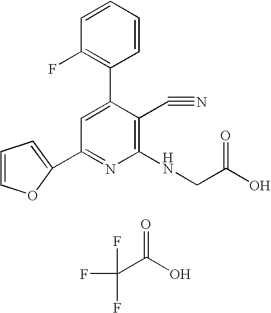 | N-[3-cyano-4-(2-fluorophenyl)-6-(2- furyl)pyridin-2-yl]glycine trifluoroacetate | 114 |
| 116 | 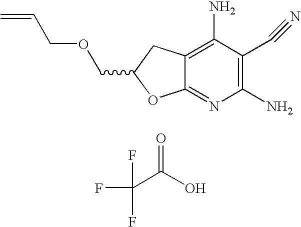 | 2-[(allyloxy)methyl]-4,6-diamino-2,3- dihydrofuro[2,3-b]pyridine-5- carbonitrile trifluoroacetate | 118 |
| 117 | 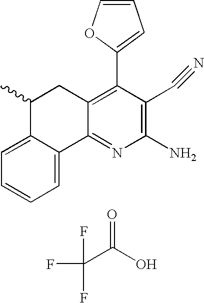 | 2-amino-4-(2-furyl)-6-methyl-5,6- dihydrobenzo[h]quinoline-3- carbonitrile trifluoroacetate | 119 |
| 118 | 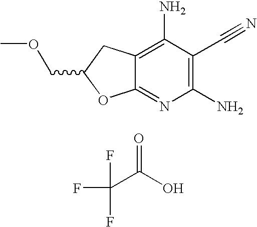 | 4,6-diamino-2-(methoxymethyl)-2,3- dihydrofuro[2,3-b]pyridine-5- carbonitrile trifluoroacetate | 119 |
| 119 | 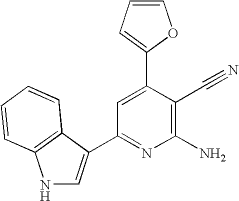 | 2-amino-4-(2-furyl)-6-(1H-indol-3- yl)nicotinonitrile | 120 |
| 120 | 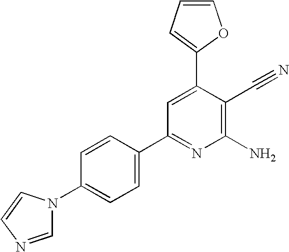 | 2-amino-4-(2-furyl)-6-[4-(1 H-imidazol- 1-yl)phenyl]nicotinonitrile | 121 |
| 121 | 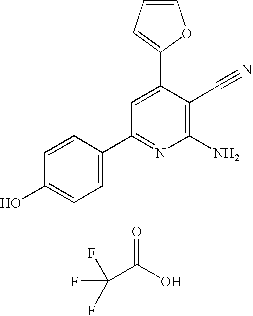 | 2-amino-4-(2-furyl)-6-(4- hydroxyphenyl)nicotinonitrile trifluoroacetate | 122 |
| 122 | 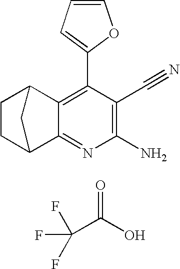 | 2-ammno-4-(2-furyl)-5,6,7,8-tetrahydro- 5,8-methanoquinoline-3-carbonitrile trifluoroacetate | 122 |
| 123 | 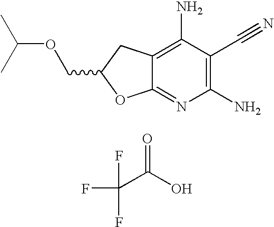 | 4,6-diamino-2-(isopropoxymethyl)-2,3- dihydrofuro[2,3-b]pyridine-5- carbonitrile trifluoroacetate | 125 |
| 124 | 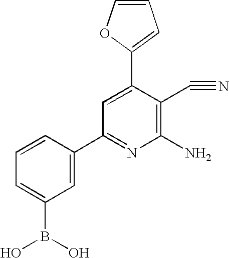 | 3-[6-amino-5-cyano-4-(2-furyl)pyridin- 2-yl]phenylboronic acid | 126 |
| 125 | 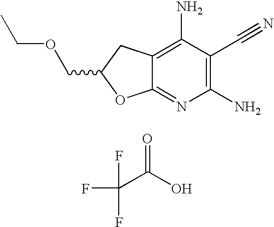 | 4,6-diamino-2-(ethoxymethyl)-2,3- dihydrofuro[2,3-b]pyridine-5- carbonitrile trifluoroacetate | 127 |
| 126 | 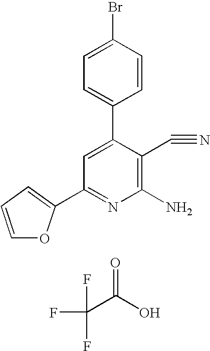 | 2-amino-4-(4-bromophenyl)-6-(2- furyl)nicotinonitnle trifluoroacetate | 130 |
| 127 | 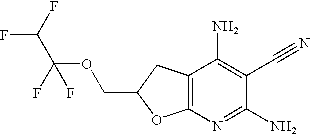 | 4,6-diamino-2-[(1,1,2,2- tetrafluoroethoxy)methyl]-2,3- dihydrofuro[2,3-b]pyridine-5- carbonitrile | 131 |
| 128 | 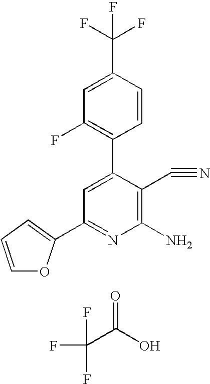 | 2-amino-4-[2-fluoro-4- (trifluoromethyl)phenyl]-6-(2- furyl)nicotinonitrile trifluoroacetate | 133 |
| 129 | 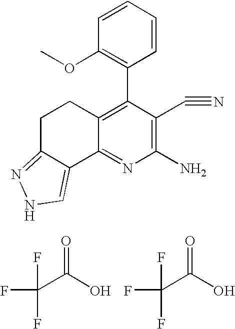 | 2-amino-4-(2-methoxyphenyl)-6,8- dihydro-5H-pyrazolo[3,4-h]quinoline-3- carbonitrile bis(trifluoroacetate) | 136 |
| 130 | 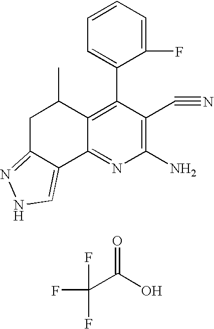 | 2-amino-4-(2-fluorophenyl)-5-methyl- 6,8-dihydro-5H-pyrazolo[3,4- h]quinoline-3-carbonitrile trifluoroacetate | 142 |
| 131 | 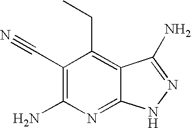 | 3,6-diamino-4-ethyl-1H-pyrazolo[3,4- b]pyridine-5-carbonitrile | 146 |
| 132 | 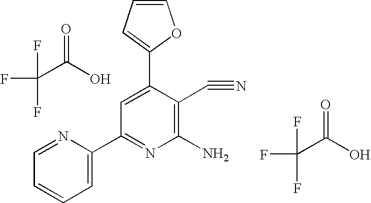 | 6-amino-4-(2-furyl)-2,2′-bipyridmne-5- carbonitrile bis(trifluoroacetate) | 149 |
| 133 | 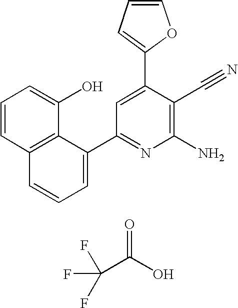 | 2-amino-4-(2-furyl)-6-(8-hydroxy-1- naphthyl)nicotinonitrile trifluoroacetate | 153 |
| 134 | 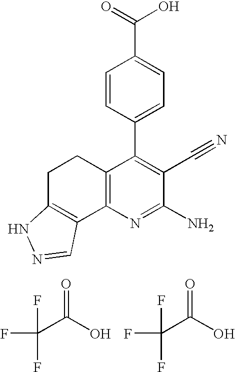 | 4-(2-amino-3-cyano-6,7-dihydro-5H- pyrazolo[3,4-h]quinolin-4-yl)benzoic acid bis(trifluoroacetate) | 155 |
| 135 | 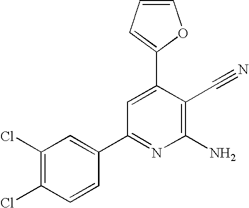 | 2-amino-6-(3,4-dichlorophenyl)-4-(2- furyl)nicotinonitrile | 156 |
| 136 | 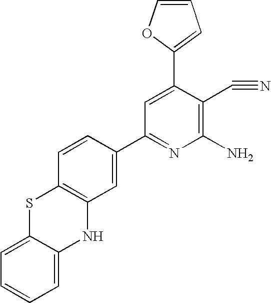 | 2-amino-4-(2-furyl)-6-(10H- phenothiazin-2-yl)nicotinonitrile | 158 |
| 137 | 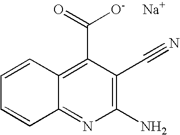 | sodium 2-amino-3-cyano-4- quinolinecarboxylate | 161 |
| 138 | 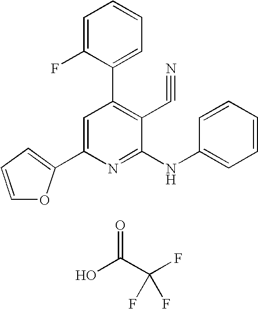 | 2-anilino-4-(2-fluorophenyl)-6-(2- furyl)nicotinonitrile | 162 |
| 139 | 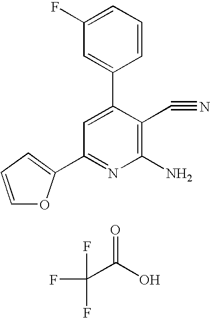 | 2-amino-4-(3-fluorophenyl)-6-(2- furyl)nicotinonitrile trifluoroacetate | 165 |
| 140 | 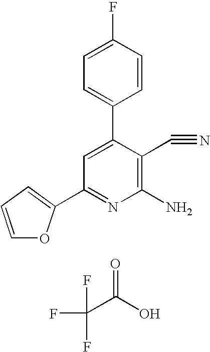 | 2-amino-4-(4-fluorophenyl)-6-(2- furyl)nicotinonitrile trifluoroacetate | 167 |
| 141 | 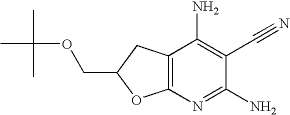 | 4,6-diamino-2-(tert-butoxymethyl)-2,3- dihydrofuro[2,3-b]pyridine-5- carbonitrile | 167 |
| 142 | 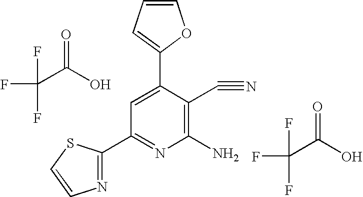 | 2-amino-4-(2-furyl)-6-(1,3-thiazol-2- yl)nicotinonitrile bis(trifluoroacetate) | 167 |
| 143 | 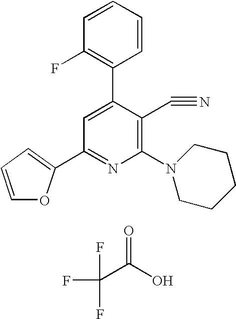 | 4-(2-fluorophenyl)-6-(2-furyl)-2- piperidin-1-ylnicotinonitrile trifluoroacetate | 176 |
| 144 | 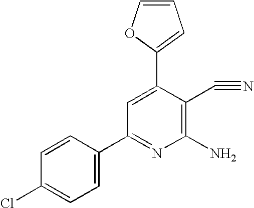 | 2-amino-6-(4-chlorophenyl)-4-(2- furyl)nicotinonitrile | 182 |
| 145 | 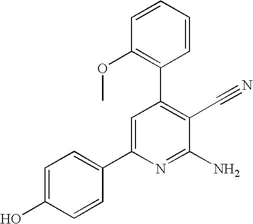 | 2-amino-6-(4-hydroxyphenyl)-4-(2- methoxyphenyl)nicotinonitrile | 183 |
| 146 | 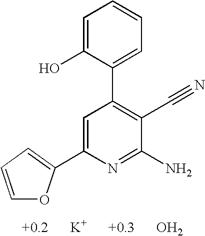 | 2-amino-6-(2-furyl)-4-(2- hydroxyphenyl)nicotinonitrile | 185 |
| 147 | 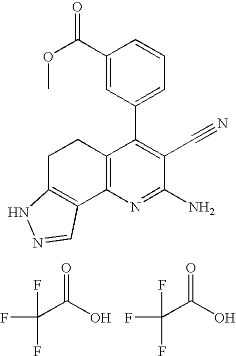 | methyl-(2-amino-3-cyano-6,7- dihydro-5H-pyrazolo[3,4-h]quinolin-4- yl)benzoate bis(trifluoroacetate) | 191 |
| 148 | 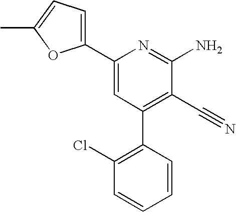 | 2-amino-4-(2-chlorophenyl)-6-(5- methyl-2-furyl)nicotinonitrile | 192 |
| 149 | 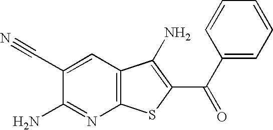 | 3,6-diamino-2-benzoylthieno[2,3- b]pyridine-5-carbonitrile | 199 |
| 150 | 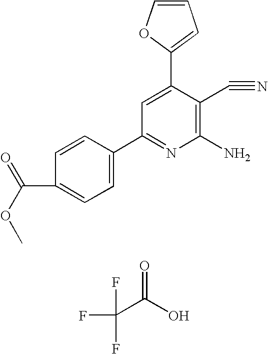 | methyl 4-[6-amino-5-cyano-4-(2- furyl)pyridin-2-yl]benzoate trifluoroacetate | 199 |
| 151 | 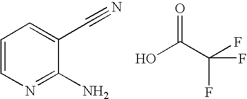 | 2-aminonicotinonitrile trifluoroacetate | 200 |
| 152 | 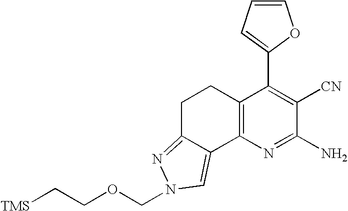 | 2-amino-4-(2-fuyl)-8-{[2- (trimethylsilyl)ethoxy]methyl}-6,8- dihydro-5H-pyrazolo[3,4-h]quinoline-3- carbonitrile | 200 |
| 153 | 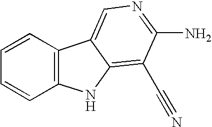 | 3-amino-5H-pyrido[4,3-b]indole-4- carbonitrile | 200 |
| 154 | 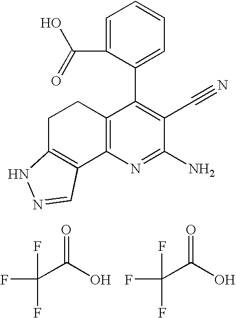 | 2-(2-amino-3-cyano-6,7-dihydro-5H- pyrazolo[3,4-h]quinolin-4-yl)benzoic acid bis(trifluoroacetate) | 200 |
| 155 | 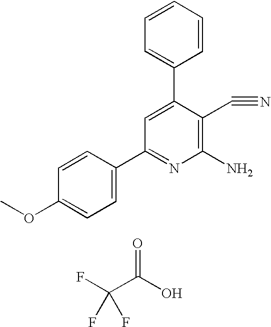 | 2-amino-6-(4-methoxyphenyl)-4- phenylnicotinonitrile trifluoroacetate | 500 |
| 156 | 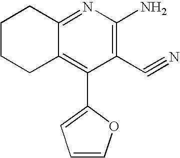 | 2-amino-4-(2-furyl)-5,6,7,8- tetrahydroquinoline-3-carbonitrile | 200 |
| 157 | 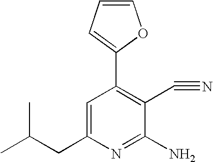 | 2-amino-4-(2-furyl)-6- isobutylnicotinonitrile | 200 |
| 158 | 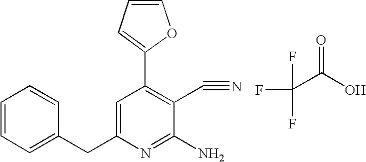 | 2-amino-6-benzyl-4-(2- furyl)nicotinonitrile trifluoroacetate | 200 |
| 159 | 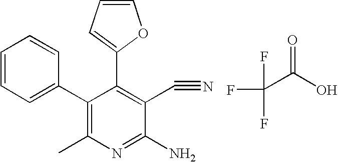 | 2-amino-4-(2-furyl)-6-methyl-5- phenylnicotinonitrile trifluoroacetate | 200 |
| 160 | 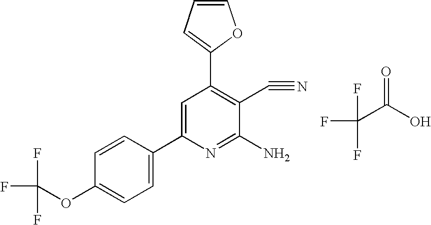 | 2-amino-4-(2-furyl)-6-[4- (trifluoromethoxy)phenyl]nicotinonitrile trifluoroacetate | 200 |
| 161 | 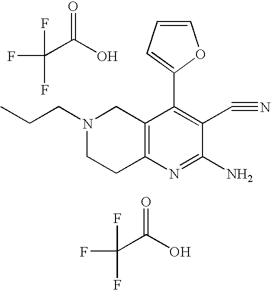 | 2-amino-4-(2-furyl)-6-propyl-5,6,7,8- tetrahydro-1,6-naphthyridine-3- carbonitrile bis(trifluoroacetate) | 200 |
| 162 | 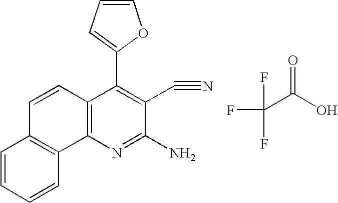 | 2-amino-4-(2-furyl)benzo[h]quinoline- 3-carbonitrile trifluoroacetate 200 | |
| 163 | 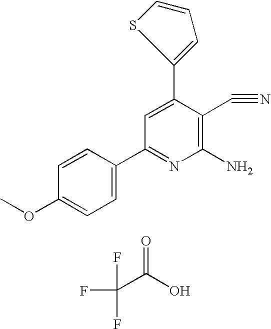 | 2-amino-6-(4-methoxyphenyl)-4-thien- 2-ylnicotinonitrile trifluoroacetate | 200 |
| 164 | 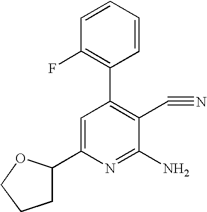 | 2-amino-4-(2-fluorophenyl)-6- tetrahydrofuran-2-ylnicotinonitrile | 200 |
| 165 | 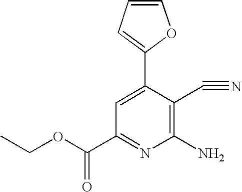 | ethyl 6-amino-5-cyano-4-(2- furyl)pyridine-2-carboxylate | 200 |
| 166 | 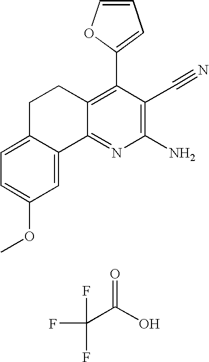 | 2-amino-4-(2-furyl)-9-methoxy-5,6- dihydrobenzo[h]quinoline-3- carbonitrile triffuoroacetate | 200 |
| 167 | 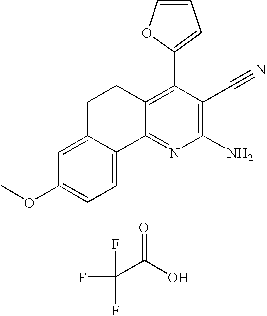 | 2-amino-4-(2-fut-yl)-8-methoxy-5,6- dihydrobenzo[h]quinoline-3- carbonitrile trifluoroacetate | 200 |
| 168 | 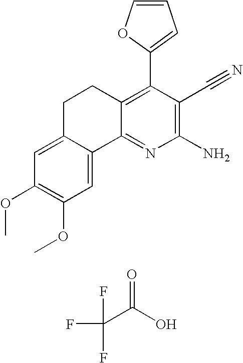 | 2-amlno-4-(2-furyl)-8,9-dimethoxy-5,6- dihydrobenzo[h]quinoline-3- carbonitrile trifluoroacetate | 200 |
| 169 | 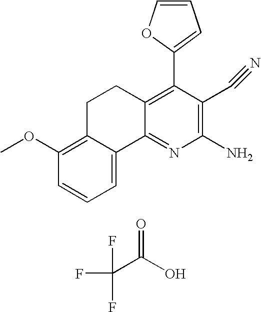 | 2-amino-4-(2-furyl)-7-methoxy-5,6- dihydrobenzo[h]quinoline-3- carbonitrile trifluoroacetate | 200 |
| 170 | 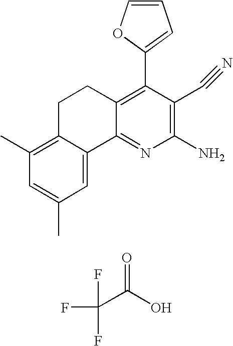 | 2-amino-4-(2-furyl)-7,9-dimethyl-5,6- dihydrobenzo[h]quinoline-3- carbonitrile trifluoroacetate | 200 |
| 171 | 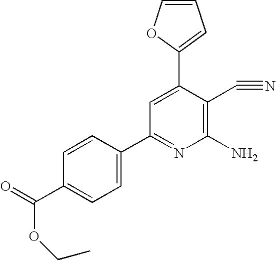 | ethyl 4-[6-amino-5-cyano-4-(2- furyl)pyridin-2-yl]benzoate | 200 |
| 172 | 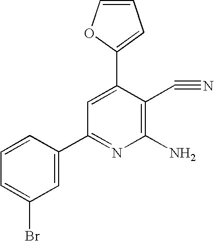 | 2-amino-6-(3-bromophenyl)-4-(2- furyl)nicotinonitrile | 200 |
| 173 | 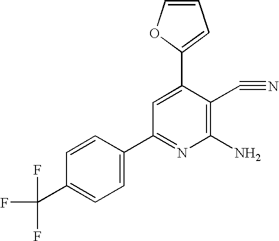 | 2-amino-4-(2-furyl)-6-[4- (trifluoromethyl)phenyl]nicotinonitrile | 200 |
| 174 | 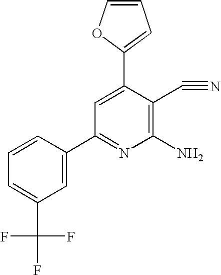 | 2-amino-4-(2-furyl)-6-[3- (trifluoromethyl)phenyl]nicotinonitrile | 200 |
| 175 | 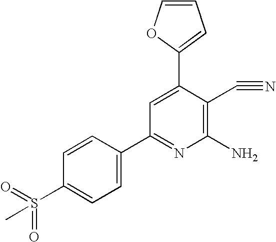 | 2-amino-4-(2-furyl)-6-[4- (methylsulfonyl)phenyl]nicotinonitrile | 200 |
| 176 | 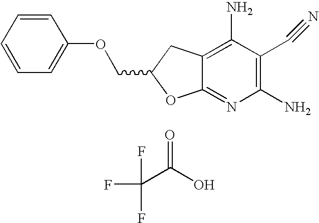 | 4,6-diamino-2-(phenoxymethyl)-2,3- dihydrofuro[2,3-b]pyridine-5- carbonitrile trifluoroacetate | 200 |
| 177 | 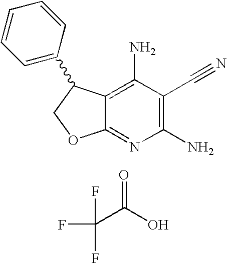 | 4,6-diamino-3-phenyl-2,3- dihydrofuro[2,3-b]pyridine-5- carbonitrile trifluoroacetate | 200 |
| 178 | 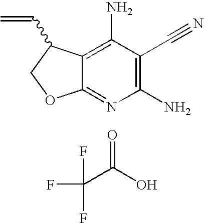 | 4,6-diamino-3-vinyl-2,3- dihydrofuro[2,3-b]pyridine-5- carbonitrile trifluoroacetate | 200 |
| 179 | 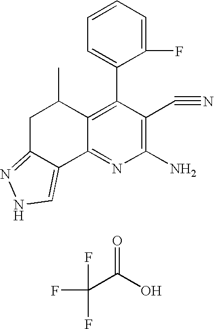 | 2-amino-4-(2-fluorophenyl)-5-methyl- 6,8-dihydro-5H-pyrazolo[3,4- h]quinoline-3-carbonitrile trifluoroacetate | 200 |
| 180 | 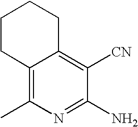 | 3-amino-1-methyl-5,6,7,8- tetrahydroisoquinoline-4-carbonitrile | 200 |
| 181 | 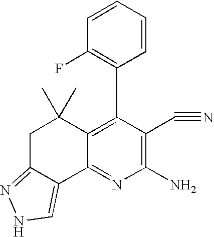 | 2-amino-4-(2-fluorophenyl)-5,5- dimethyl-6,8-dihydro-5H-pyrazolo[3,4- h]quinoline-3-carbonitrile | 200 |
| 182 | 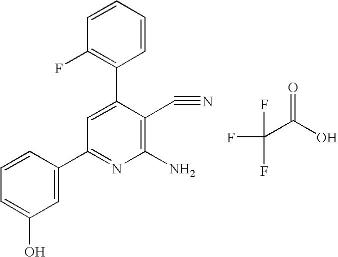 | 2-amino-4-(2-fluorophenyl)-6-(3- hydroxyphenyl)nicotinonitrile trifluoroacetate | 200 |
| 183 | 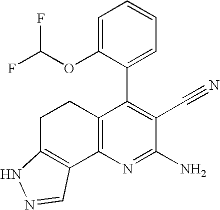 | 2-amino-4-[2-(difluoromethoxy)phenyl]6,7-dihydro-5H-pyrazolo[3,4- h]quinoline-3-carbonitrile | 200 |
| 184 | 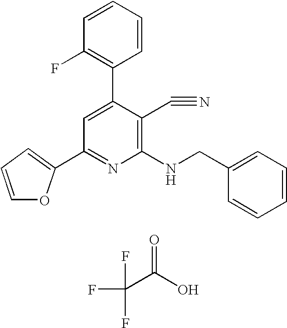 | 2-(benzylamino)-4-(2-fluorophenyl)-6- (2-furyl)nicotinonitrile trifluoroacetate | 200 |
| 185 | 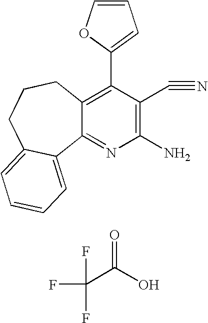 | 2-amino-4-(2-furyl)-6,7-dihydro-5H- benzo[6,7]cyclohepta[1,2-b]pyridtne-3- carbonitrile trifluoroacetate | 200 |
| 186 | 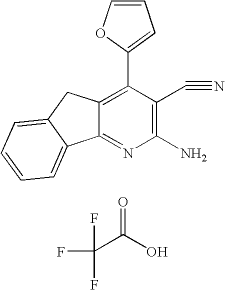 | 2-amino-4-(2-furyl)-5H-indeno[1,2- b]pyridine-3-carbonitrile trifluoroacetate | 200 |
| 187 | 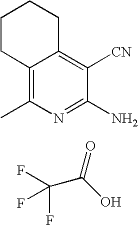 | 3-amino-1-methyl-5,6,7,8- tetrahydroisoquinoline-4-carbonitrile trifluoroacetate | 200 |
| 188 | 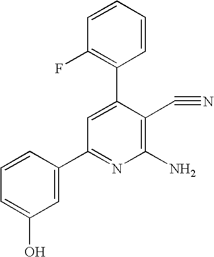 | 2-amino-4-(2-fluorophenyl)-6-(3- hydroxyphenyl)nicotinonitrile | 200 |
| 189 | 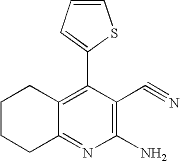 | 2-amino-4-(2-thienyl)-5,6,7,8- tetrahydro-3-quinolinecarbonitrile | 200 |
| 190 | 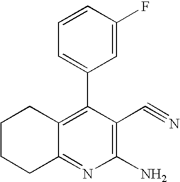 | 2-amino-4-(3-fluorophenyl)-5,6,7,8- tetrahydro-3-quinolinecarbonitrile | 200 |
| 191 | 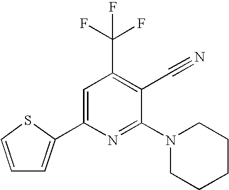 | 2-(1-piperidinyl)-6-(2-thienyl)-4- (trifluoromethyl)nicotinonitrile | 200 |
| 192 | 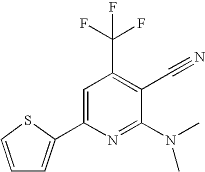 | 2-(dimethylamino)-6-(2-thienyl)-4- (trifluoromethyl)nicotinonitrile | 200 |
| 193 | 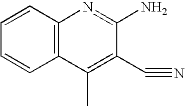 | 3-Quinolinecarbonitrile, 2-amino-4- methyl- or 2-amino-4-methyl-3- quinolinecarbonitrile | 200 |
| 194 | 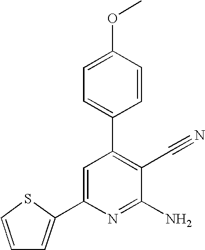 | 2-amino-4-(4-methoxyphenyl)-6-(2- thienyl)nicotinonitrile | 200 |
| 195 | 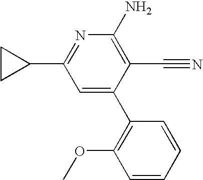 | 2-amino-6-cyclopropyl-4-(2- methoxyphenyl)nicotinonitrile | 200 |
| 196 | 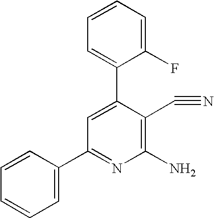 | 2-amino-4-(2-fluorophenyl)-6- phenylnicotinonitrile | 200 |
| 197 | 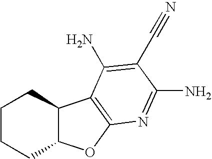 | (4bS,8aR)-2,4-diamino-4b,5,6,7,8,8a- hexahydro[1 ]benzofuro[2,3-b]pyridine- 3-carbonitrile | 200 |
| 198 | 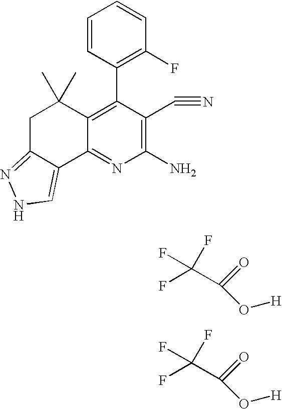 | 2-amino-4-(2-fluorophenyl)-5,5- dimethyl-6,8-dihydro-5H-pyrazolo[3,4- h]quinoline-3-carbonitrile bis(trifluoroacetate) | 200 |
| 199 | 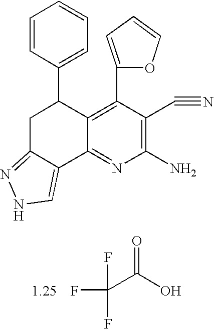 | 2-amino-4-(2-furyl)-5-phenyl-6,8- dihydro-5H-pyrazolo[3,4-h]quinoline-3- carbonitrile trifluoroacetate | 200 |
| 200 | 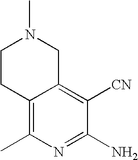 | 3-amino-1,6-dimethyl-5,6,7,8- tetrahydro-2,6-naphthyridine-4- carbonitrile | 200 |
| 201 | 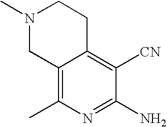 | 3-amino-1,7-dimethyl-5,6,7,8- tetrahydro-2,7-naphthyridine-4- carbonitrile | 200 |
| 202 | 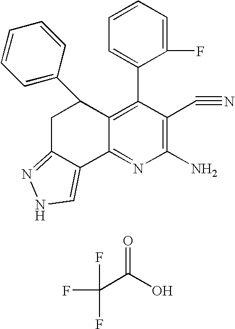 | 2-amino-4-(2-fluorophenyl)-5-phenyl- 6,8-dihydro-5H-pyrazolo[3,4- h]quinoline-3-carbonitrile trifluoroacetate | 200 |
| 203 | 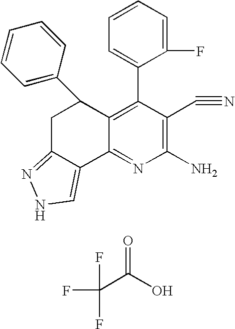 | 2-amino-4-(2-fluorophenyl)-5-phenyl- 6,8-dihydro-5H-pyrazolo[3,4- h]quinoline-3-carbonitrile trifluoroacetate | 200 |
| 204 | 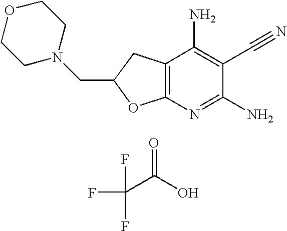 | 4,6-diamino-2-(morpholin-4-ylmethyl)- 2,3-dihydrofuro[2,3-b]pyridine-5- carbonitrile | 200 |
| 205 | 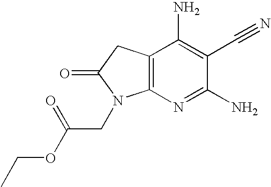 | ethyl (4,6-diamino-5-cyano-2-oxo-2,3- dihydro-1H-pyrrolo[2,3-b]pyridin-1- yl)acetate | 200 |
| 206 | 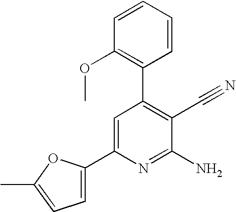 | 2-amino-4-(2-methoxyphenyl)-6-(5- methyl-2-furyl)nicotinonitrile | 200 |
| 207 | 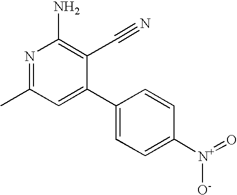 | 2-amino-6-methyl-4-(4- nitrophenyl)nicotinonitrile | 200 |
| 208 | 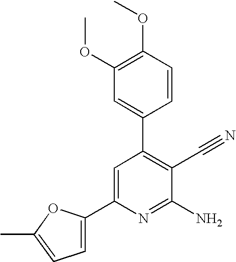 | 2-amino-4-(3,4-dimethoxyphenyl)-6-(5- methyl-2-furyl)nicotinonitrile | 200 |
| 209 | 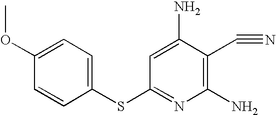 | 2,4-diamino-6-[(4- methoxyphenyl)thio]nicotinonitrile | 200 |
| 210 | 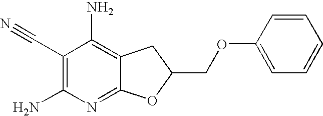 | 4,6-diamino-2-(phenoxymethyl)-2,3- dihydrofuro[2,3-b]pyridine-5- carbonitrile | 200 |
| 211 | 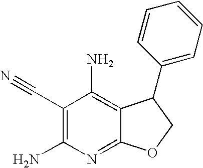 | 4,6-diamino-3-phenyl-2,3- dihydrofuro[2,3-b]pyridine-5- carbonitrile | 200 |
| 212 | 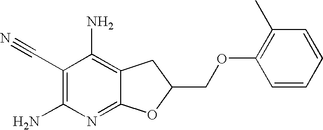 | 4,6-diamino-2-[(2- methylphenoxy)methyl]-2,3- dihydrofuro[2,3-b]pyridine-5- carbonitrile | 200 |
| 213 | 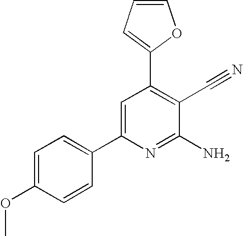 | 2-amino-4-(2-furyl)-6-(4- methoxyphenyl)nicotinonitrile | 200 |
| 214 | 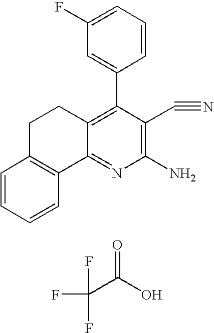 | 2-amino-4-(3-fluorophenyl)-5,6- dihydrobenzo[h]quinoline-3- carbonitrile trifluoroacetate | 200 |
| 215 | 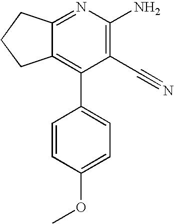 | 2-amino-4-(4-methoxyphenyl)-6,7- dihydro-5H-cyclopenta[b]pyridine-3- carbonitrile | 200 |
| 216 | 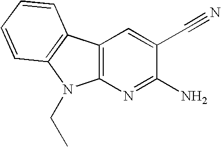 | 2-amino-9-ethyl-9H-pyrido[2,3- b]indole-3-carbonitrile | 200 |
| 217 | 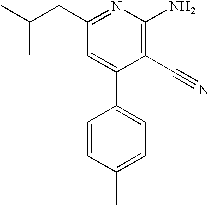 | 2-amino-6-Isobutyl-4-(4- methylphenyl)nicotinonitrile | 200 |
| 218 | 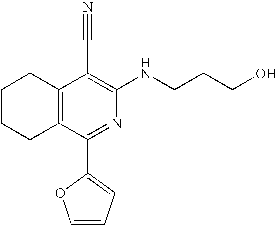 | 1-(2-furyl)-3-[(3-hydroxypropyl)amino]- 5,6,7,8-tetrahydroisoquinoline-4- carbonitrile | 200 |
| 219 | 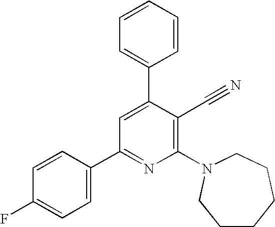 | 2-azepan-1-yl-6-(4-fluorophenyl)-4- phenylnicotinonitrile | 200 |
| 220 | 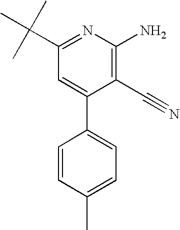 | 2-amino-6-tert-butyl-4-(4- methylphenyl)nicotinonitrile | 200 |
| 221 | 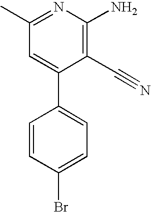 | 2-amino-4-(4-bromophenyl)-6- methylnicotinonitrile | 200 |
| 222 | 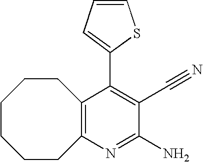 | 2-amino-4-thien-2-yl-5,6,7,8,9,10- hexahydrocycloocta[b]pyridine-3- carbonitrile | 200 |
| 223 | 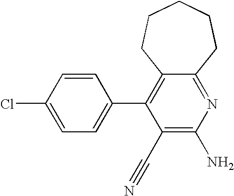 | 2-amino-4-(4-chlorophenyl)-6,7,8,9- tetrahydro-5H-cyclohepta[b]pyridine-3- carbonitrile | 200 |
| 224 | 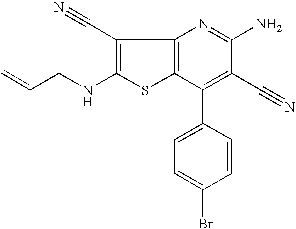 | 2-(allylamino)-5-amino-7-(4- bromophenyl)thieno[3,2-b]pyridine-3,6- dicarbonitrile | 200 |
| 225 | 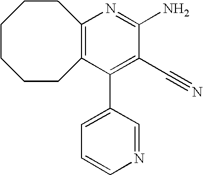 | 2-amino-4-pyridin-3-yl-5,6,7,8,9,10- hexahydrocycloocta[b]pyridine-3- carbonitrile | 200 |
| 226 | 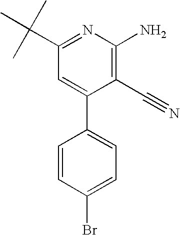 | 2-amino-4-(4-bromophenyl)-6-tert- butylnicotinonitrile | 200 |
| 227 | 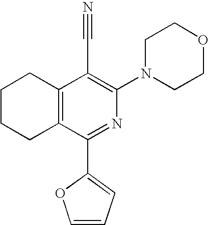 | 1-(2-furyl)-3-morpholin-4-yl-5,6,7,8- tetrahydroisoquinoline-4-carbonitrile | 200 |
| 228 | 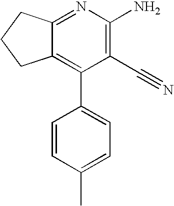 | 2-amino-4-(4-methylphenyl)-6,7- dihydro-5H-cyclopenta[b]pyridine-3- carbonitrile | 200 |
| 229 | 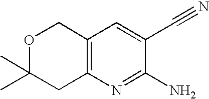 | 2-amino-7,7-dimethyl-7,8-dihydro-5H- pyrano[4,3-b]pyridine-3-carbonitrile | 200 |
| 230 | 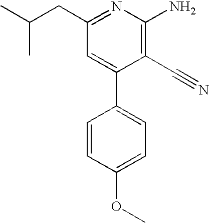 | 2-amino-6-isobutyl-4-(4- methoxyphenyl)nicotinonitrile | 200 |
| 231 | 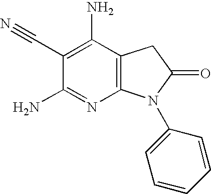 | 4,6-diamino-2-oxo-1-phenyl-2,3- dihydro-1H-pyrrolo[2,3-b]pyridine-5- carbonitrile | 200 |
| 232 | 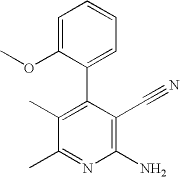 | 2-amino-4-(2-methoxyphenyl)-5,6- dimethylnicotinonitrile | 200 |
| 233 | 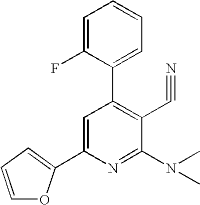 | 2-(dimethylamino)-4-(2-fluorophenyl)- 6-(2-furyl)nicotinonitrile | 200 |
| 234 | 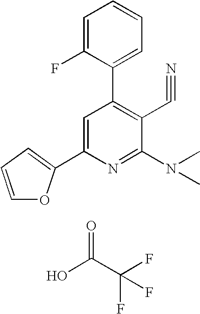 | 2-(dimethylamino)-4-(2-fluorophenyl)- 6-(2-furyl)nicotinonitrile | 200 |
| 235 | 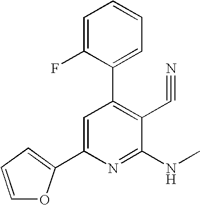 | 4-(2-fluorophenyl)-6-(2-furyl)-2- (methylamino)nicotinonitrile | 200 |
| 236 | 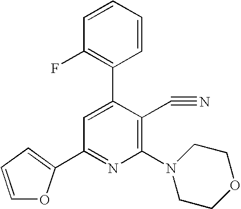 | 4-(2-fluorophenyl)-6-(2-furyl)-2- morpholin-4-ylnicotinonitrile | 200 |
| 237 | 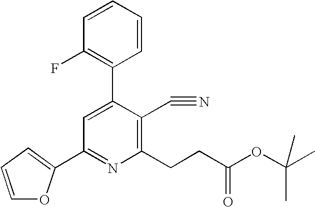 | tert-butyl N-[3-cyano-4-(2- fluorophenyt)-6-(2-furyl)pyridin-2- yl]glycinate | 200 |
| 238 | 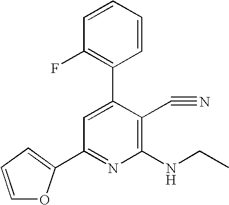 | 2-(ethylamino)-4-(2-fluorophenyl)-6-(2- furyl)nicotinonitrile | 200 |
| 239 | 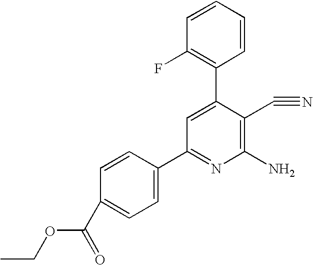 | ethyl 4-[6-amino-5-cyano-4-(2- fluorophenyl)pyridin-2-yl]benzoate | 200 |
| 240 | 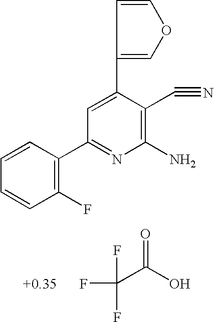 | 2-amino-6-(2-fluorophenyl)-4-(3- furyl)nicotinonitrile trifluoroacetate | 200 |
| 241 | 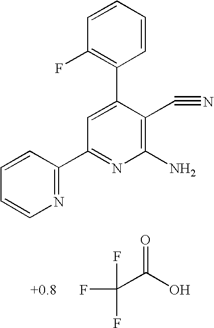 | 6-amino-4-(2-fluorophenyl)-2,2′- bipyridine-5-carbonitrile trifluoroacetate | 200 |
| 242 | 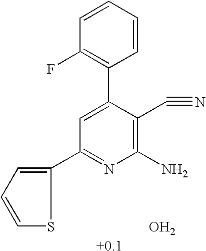 | 2-amino-4-(2-fluorophenyl)-6-thien-2- ylnicotinonitrile hydrate | 200 |
| 243 | 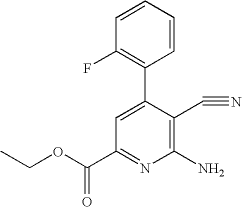 | ethyl 6-amino-5-cyano-4-(2- fluorophenyl)pyridine-2-carboxylate | 200 |
| 244 | 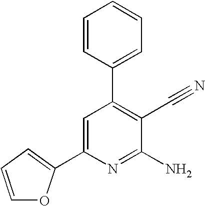 | 2-amino-6-(2-furyl)-4- phenylnicotinonitrile | 200 |
| 245 | 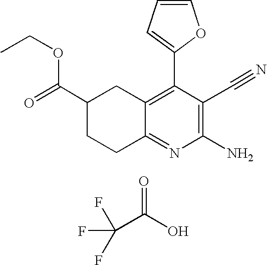 | ethyl 2-amino-3-cyano-4-(2-furyl)- 5,6,7,8-tetrahydroquinoline-6- carboxylate trifluoroacetate | 200 |
| 246 | 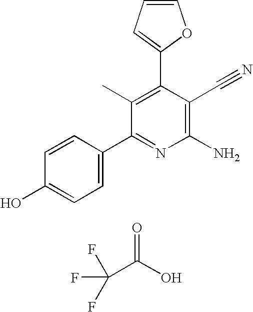 | 2-amino-4-(2-furyl)-6-(4- hydroxyphenyl)-5-methylnicotinonitrile trifluoroacetate | 200 |
| 247 | 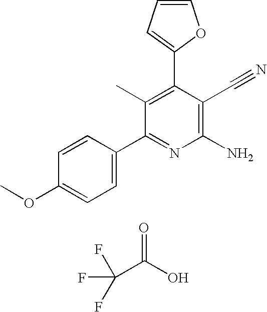 | 2-amino-4-(2-furyl)-6-(4- methoxyphenyl)-5- methylnicotinonitrile trifluoroacetate | 200 |
| 248 | 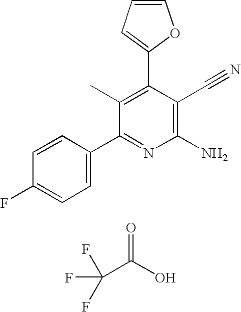 | 2-amino-6-(4-fluorophenyl)-4-(2-furyl)- 5-methylnicotinonitrile trifluoroacetate | 200 |
| 249 | 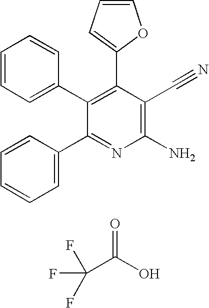 | 2-amino-4-(2-furyl)-5,6- diphenylnicotinonitrile trifluoroacetate | 200 |
| 250 | 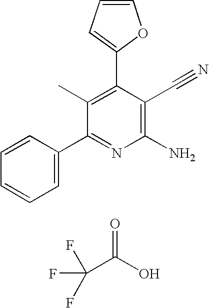 | 1 2-amino-4-(2-furyl)-5-methyl-6- phenylnicotinonitrile trifluoroacetate | 200 |
| 251 | 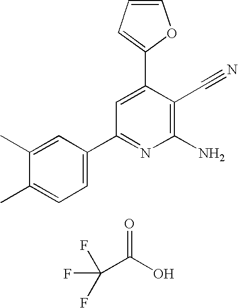 | 2-amino-6-(3,4-dimethylphenyl)-4-(2- furyl)nicotinonitrile trifluoroacetate | 200 |
| 252 | 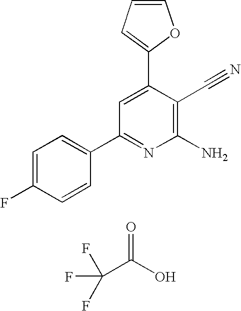 | 2-amino-6-(4-fluorophenyl)-4-(2- furyl)nicotinonitrile trifluoroacetate | 200 |
| 253 | 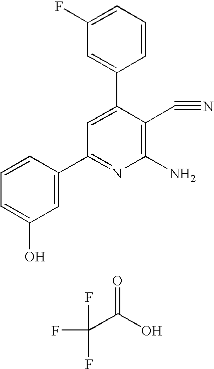 | 2-amino-4-(3-fluorophenyl)-6-(3- hydroxyphenyl)nicotinonitrile trifluoroacetate | 200 |
| 254 | 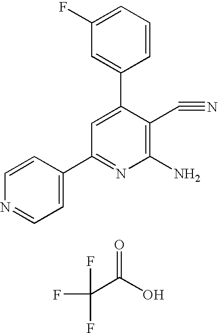 | 6-amino-4-(3-fluorophenyl)-2,4- bipyridine-5-carbonitrile trifluoroacetate | 200 |
| 255 | 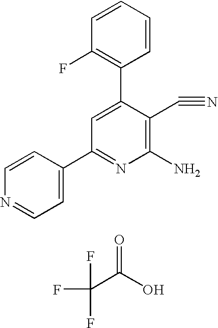 | 6-amino-4-(2-fluorophenyl)-2,4- bipyridine-5-carbonitrile trifluoroacetate | 200 |
| 256 | 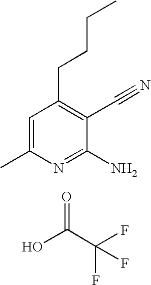 | 2-amino-4-butyl-6-methylnicotinonitrile trifluoroacetate | 200 |
| 257 | 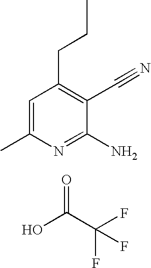 | 2-amino-6-methyl-4- propylnicotinonitrile trifluoroacetate | 200 |
| 258 | 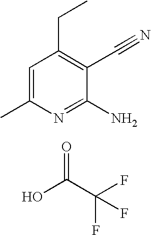 | 2-amino-4-ethyl-6-methylnicotinonitrile trifluoroacetate | 200 |
| 259 | 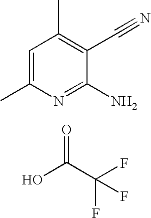 | 2-amino-4,6-dimethylnicotinonitriie trifluoroacetate | 200 |
| 260 | 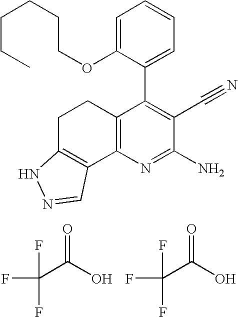 | 2-amino-4-[2-(hexyloxy)phenyl]-6,7- dihydro-5H-pyrazolo[3,4-h]quinoline-3- carbonitrile bis(trifluoroacetate) | 200 |
| 261 | 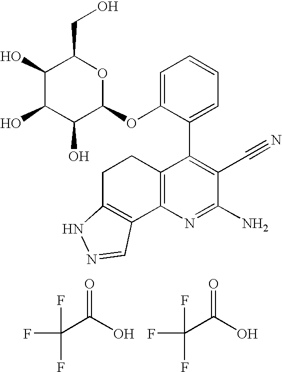 | 2-amino-4-[2-(beta-D- glucopyranosyloxy)phenyl]-6,7- dihydro-5H-pyrazolo[3,4-h]quinoline-3- carbonitrile bis(trifluoroacetate) | 200 |
| 262 | 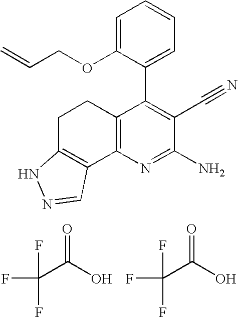 | 4-[2-(allyloxy)phenyl]-2-amino-6,7- dihydro-5H-pyrazolo[3,4-h]quinoline-3- carbonitrile bis(trifluoroacetate) | 200 |
| 263 | 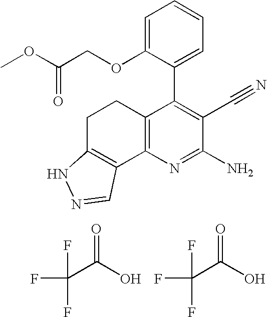 | methyl [2-(2-amino-3-cyano-6,7- dihydro-5H-pyrazolo[3,4-h]quinolin-4- yl)phenoxy]acetate bis(trifluoroacetate) | 200 |
| 264 | 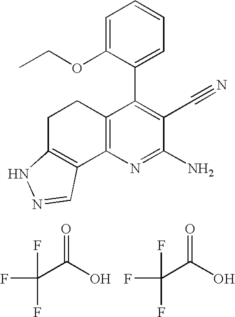 | 2-amino-4-(2-ethoxyphenyl)-6,7- dihydro-5H-pyrazolo[3,4-h]quinoline-3- carbonitrile bis(trifluoroacetate) | 200 |
| 265 | 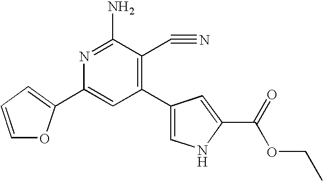 | ethyl 4-[2-amino-3-cyano-6-(2- furyl)pyridin-4-yl]-1H-pyrrole-2- carboxylate | 200 |
| 266 | 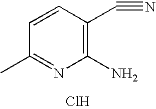 | 2-amino-6-methylnicotinonitrile hydrochloride | 200 |
| 267 | 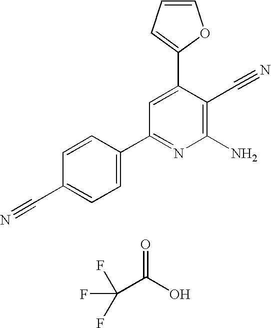 | 2-amino-6-(4-cyanophenyl)-4-(2- furyl)nicotinonitrile trifluoroacetate | 200 |
| 268 | 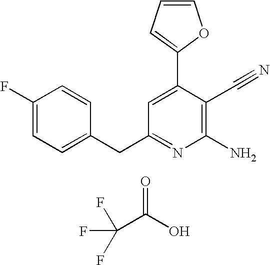 | 2-amino-6-(4-fluorobenzyl)-4-(2- furyl)nicotinonitrile trifluoroacetate | 200 |
| 269 | 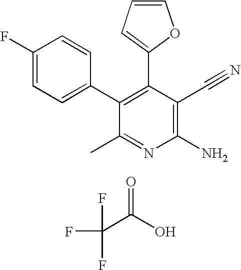 | 2-amino-5-(4-fluorophenyl)-4-(2- furyl)-6-methylnicotinonitrile trifluoroacetate | 200 |
| 270 | 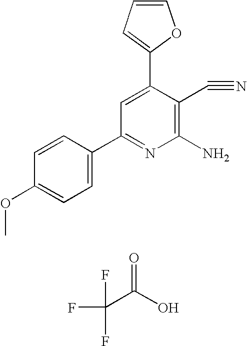 | 2-amino-4-(2-furyl)-6-(4- methoxyphenyl)nicotinonitrile trifluoroacetate | 200 |
| 271 | 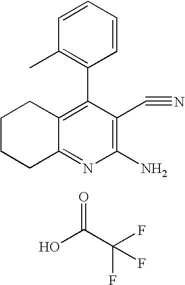 | 2-amino-4-(2-methylphenyl)-5,6,7,8- tetrahydroquinoline-3-carbonitrile trifluoroacetate | 200 |
| 272 | 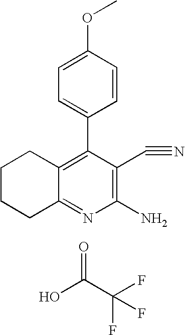 | 2-amino-4-(4-methoxyphenyl)-5,6,7,8 tetrahydroquinoline-3-carbonitrile trifluoroacetate | 200 |
| 273 | 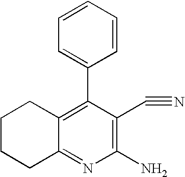 | 2-amino-4-phenyl-5,6,7,8- tetrahydroquinoline-3-carbonitrile | 200 |
| 274 | 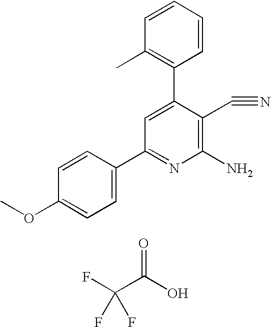 | 2-amino-6-(4-methoxyphenyl)-4-(2- methylphenyl)nicotinonitrile trifluoroacetate | 200 |
| 275 | 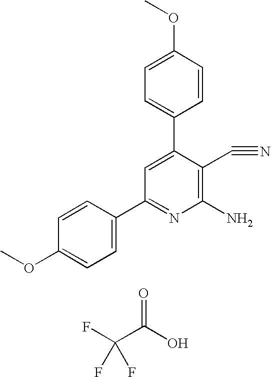 | 2-amino-4,6-bis(4- methoxyphenyl)nicotinonitrile trifluoroacetate | 200 |
| 276 | 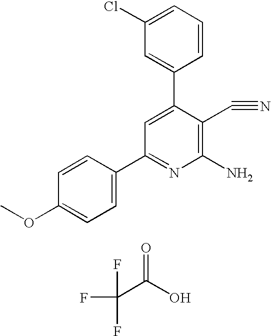 | 2-amino-4-(3-chlorophenyl)-6-(4- methoxyphenyl)nicotinonitrile trifluoroacetate | 200 |
| 277 | 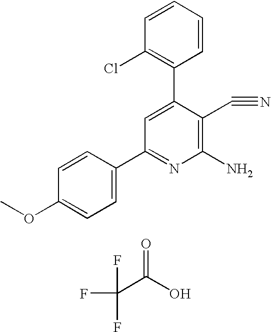 | 2-amino-4-(2-chlorophenyl)-6-(4- methoxyphenyl)nicotinonitrile trifluoroacetate | 200 |
| 278 | 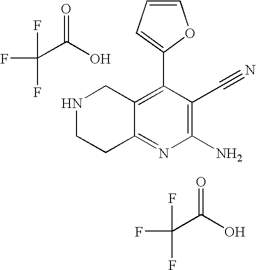 | 2-amino-4-(2-furyl)-5,6,7,8- tetrahydro-1,6-naphthyridine-3- carbonitrile bis(trifluoroacetate) | 200 |
| 279 | 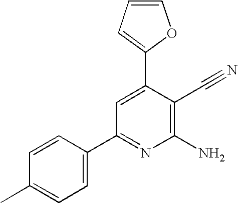 | 2-amino-4-(2-furyl)-6-(4- methylphenyl)nicotinonitrile | 200 |
| 280 | 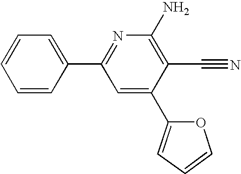 | 2-amino-4-(2-furyl)-6- phenylnicotinonitrile | 200 |
| 281 | 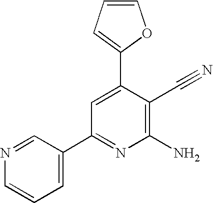 | 6-amino-4-(2-furyl)-2,3-bipyridine-5- carbonitrile | 200 |
| 282 | 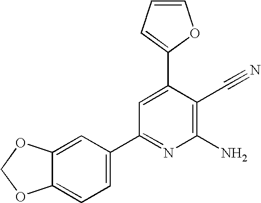 | 2-amino-6-(1,3-benzodioxol-5-yl)-4-(2- furyl)nicotinonitrile | 200 |
| 283 | 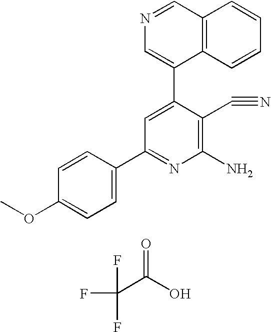 | 2-amino-4-isoquinolin-4-yl-6-(4- methoxyphenyl)nicotinonitrile trifluoroacetate | 200 |
| 284 | 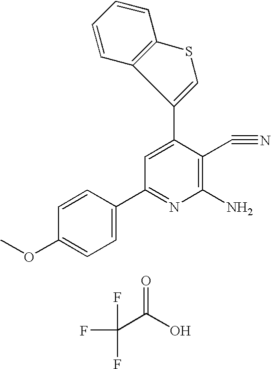 | 2-amino-4-(1-benzothien-3-yl)-6-(4- methoxyphenyl)nicotinonitrile trifluoroacetate | 200 |
| 285 | 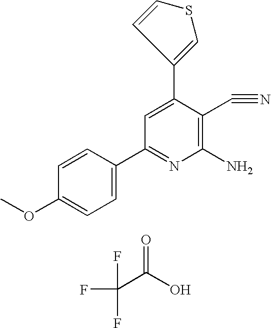 | 2-amino-6-(4-methoxyphenyl)-4-thien- 3-ylnicotinonitrile trifluoroacetate | 200 |
| 286 | 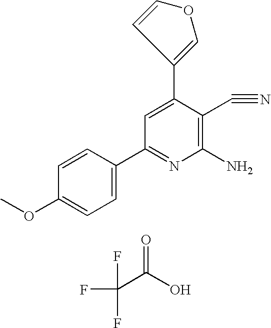 | 2-amino-4-(3-furyl)-6-(4- methoxyphenyl)nicotinonitrile trifluoroacetate | 200 |
| 287 | 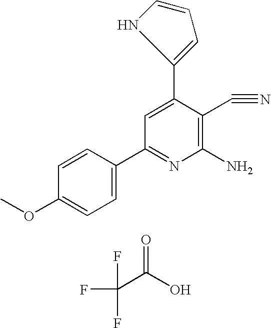 | 2-amino-6-(4-methoxyphenyl)-4-(1H- pyrrol-2-yl)nicotinonitrile trifluoroacetate | 200 |
| 288 | 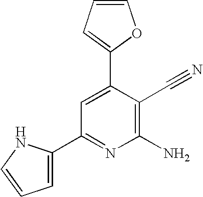 | 2-amino-4-(2-furyl)-6-(1H-pyrrol-2- yl)nicotinonitrile | 200 |
| 289 | 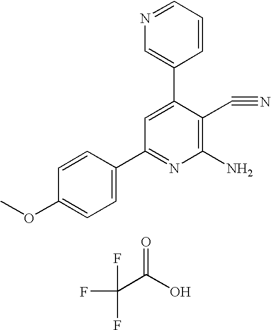 | 2-amino-6-(4-methoxyphenyl)-3,4′- bipyridine-3′-carbonitrile trifluoroacetate | 200 |
| 290 | 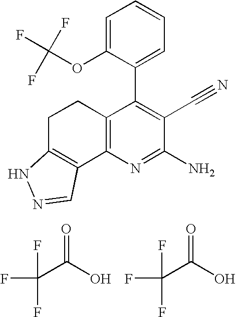 | 2-amino-4-[2- (trifluoromethoxy)phenyl]-6,7-dihydro- 5H-pyrazolo[3,4-h]quinoline-3- carbonitrile bis(trifluoroacetate) | 200 |
| 291 | 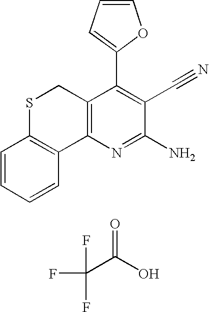 | 2-amino-4-(2-furyl)-5H- thiochromeno[4,3-b]pyridine-3- carbonitrile trifluoroacetate | 200 |
| 292 | 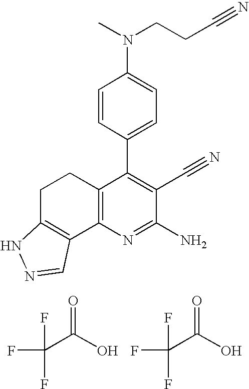 | 2-amino-4-{4-[(2- cyanoethyl)(methyl)amino]phenyl}-6,7- dihydro-5H-pyrazolo[3,4-h]quinoline-3- carbonitrile bis(trifluoroacetate) | 200 |
| 293 | 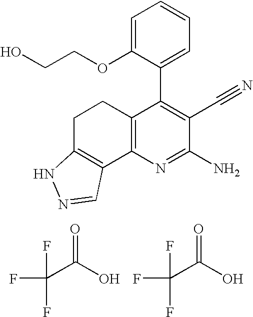 | 2-amino-4-[2-(2- hydroxyethoxy)phenyl]-6,7-dihydro-5H- pyrazolo[3,4-h]quinoline-3-carbonitrile bis(trifluoroacetate) | 200 |
| 294 | 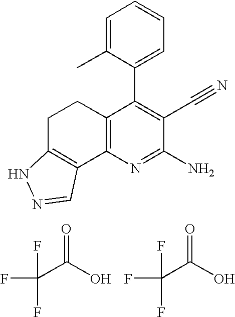 | 2-amino-4-(2-methylphenyl)-6,7- dihydro-5H-pyrazolo[3,4-h]quinoline-3- carbonitrile bis(trifluoroacetate) | 200 |
| 295 | 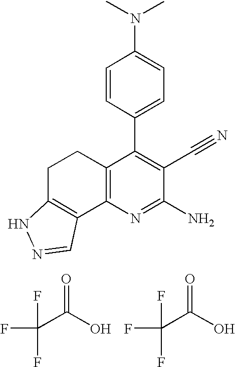 | 2-amino-4-[4-(dimethylamino)phenyl]- 6,7-dihydro-5H-pyrazolo[3,4- h]quinoline-3-carbonitrile bis(trifluoroacetate) | 200 |
| 296 | 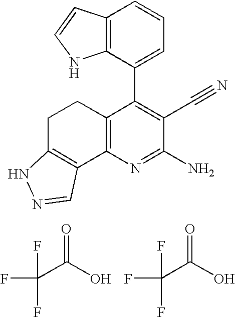 | 2-amino-4-(1H-indol-7-yl)-6,7-dihydro- 5H-pyrazolo[3,4-h]quinoline-3- carbonitrile bis(trifluoroacetate) | 200 |
| 297 | 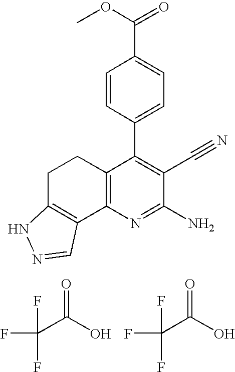 | methyl 4-(2-amino-3-cyano-6,7- dihydro-5H-pyrazolo[3,4-h]quinolin-4- yl)benzoate bis(trifluoroacetate) | 200 |
| 298 | 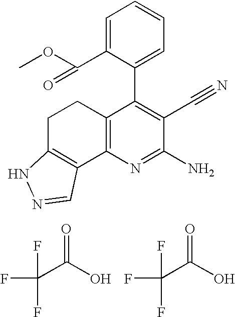 | methyl 2-(2-amino-3-cyano-6,7- dihydro-5H-pyrazolo[3,4-h]quinolin-4- yl)benzoate bis(trifluoroacetate) | 200 |
| 299 | 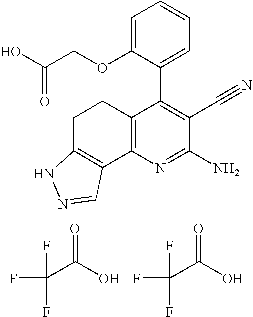 | [2-(2-amino-3-cyano-6,7-dihydro-5H- pyrazolo[3,4-h]quinolin-4- yl)phenoxy]acetic acid bis(trifluoroacetate) | 200 |
| 300 | 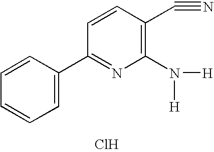 | 2-amino-6-phenylnicotinonitrile hydrochloride | 200 |
| 301 | 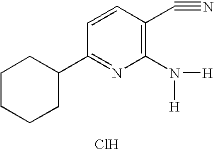 | 2-amino-6-cyclohexylnicotinonitrile hydrochloride | 200 |
| 302 | 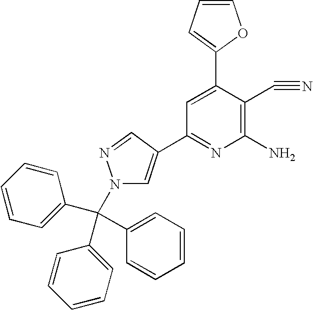 | 2-amino-4-(2-furyl)-6-(1-trityl-1H- pyrazol-4-yl)nicotinonitrile | 200 |
| 303 | 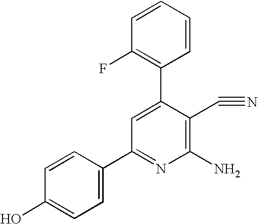 | 2-amino-4-(2-fluorophenyl)-6-(4- hydroxyphenyl)nicotinonitrile | 200 |

Notes:
^{a}The aminocyanopyridine compound may be shown with a solvent, such as, for example, trifluoroacetate, with which it can form a salt. Both the salt and acid forms of the aminocyanopyridine compound are included in the present invention.
^{b}Compound names generated by AGO/Name software.

In another embodiment, the present invention comprises an aminocyanopyridine compound having the structure shown in formula I, where:

R¹ is selected from the group consisting of —H, methyl, ethyl, propyl, butyl, —(CH₂)COOH, phenyl, pyridyl, dimethylaminoethyl, methoxyethyl, tetramethylaminoethyl, carboxymethyl, and phenylacetyl;

R² is selected from the group consisting of —H, methyl, ethyl, propyl, butyl, amino, phenyl, methoxy, carboxy, carboxymethyl, hydroxyethylamino, propylamino, ethylamino, methylamino, methoxyethyl, ethoxyethylamino, aminoethylamino, benzylamino, dimethylaminoethylamino, phthaloaminoethyl, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), tetramethylaminoethyl, acetylaminophenyl, methoxyphenylamino, carboxyphenyl, carboxy-3-isopyrryl, cyanophenyl, cyclopropyl, phenoxyphenyl, pyridyl, dihydroxybromophenyl, difluoromethoxyphenyl, trifluoromethylphenyl, trifluoromethylfluorophenyl, hydroxyphenyl, methylaminomethyl, methylaminoethyl, thiophyl, pyrryl, aminomethyl,

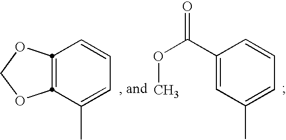

R³ is selected from the group consisting of —H, methyl, ethyl, propyl, isopropyl, cyano, aminomethyl, phenyl, fluorophenyl, and amino, except that when R² is heteroaryl, R³ is other than cyano;

wherein the R² and R³ groups are such that they optionally join to form a ring system selected from:

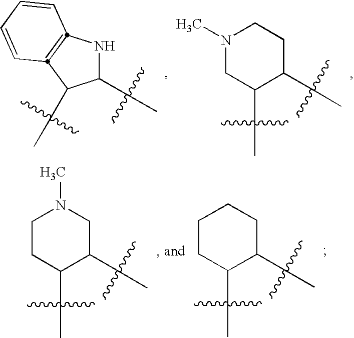

R⁴ is selected from the group consisting of —H, methyl, ethyl, propyl, hydroxy, furyl, methylfuryl, methylimidazolyl, phenyl, hydroxyphenyl, carboxyphenyl, pyrazolyl, hydroxy, dihydroxyphenyl, methoxyphenyl, chlorophenyl, bromophenyl, fluorophenyl, dichlorophenyl, dihydroxyborophenyl, thienyl, pyrryl, N-methylpyrryl, pyridyl, methylthio, methylsulfonylphenyl, carboethoxyphenyl, methoxy, carbamylphenyl, mercapto, N-isoimidazoylphenyl, isopropyl, amino, hydroxynaphthyl, thiazoyl, carboxymethylphenyl, trifluoromethylphenyl, methylphenyl, cyanophenyl, dimethylphenyl, fluorobenzhydryl, methoxyfuryl, aminosulfonylphenyl,

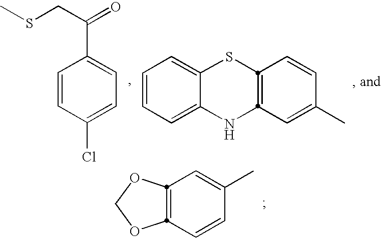

wherein the R³ and R⁴ groups are such that they optionally join to form a ring system selected from:

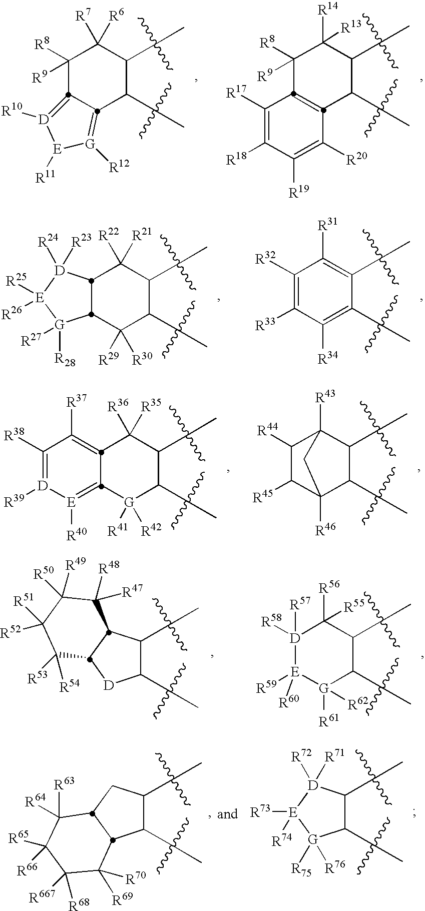

In preferred embodiments, when R⁴ is pyridine, thiophene, or phenyl, it is substituted, if at all, with a substituent group that is other than hydroxyl;

D, E and G are each independently selected from the group consisting of carbon, oxygen, sulfur, and nitrogen;

R⁵ is selected from the group consisting of —H, and C₁-C₅ alkyl, provided that at least one of R¹, R², R³, R⁴, and R⁵ is other than hydrogen; and

wherein the R¹ and R⁵ groups optionally join to form a piperidyl ring;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, methyl, ethyl, propyl, butyl, isobutyl, amino, nitro, hydroxy, methoxy, ethoxy, propoxy, 2-propenoxy, oxo, carboxy, bromo, chloro, fluoro, trifluoromethyl, chloromethyl, hydroxymethyl, dicyanomethyl, 2-fluorophenyl, 3-fluorophenyl, hydroxyethoxy, ethoxyethoxy, —(CH₂)—O—(C₆H₄)—O—(CH₃), carboxymethoxy, isopropylcarboxymethoxy, isobutylcarboxymethoxy, methylamino, dimethylamino, aminoethoxy, diaminoethoxy, dimethylaminoethoxy, cyanomethoxymethyl, 2-propenoxymethyl, methoxymethyl, isopropoxymethyl, ethoxymethyl, —(CH₂)—O—(CF₂)—CHF₂, isobutoxymethyl, benzoyl, phenyl, N-morpholinyl, morpholinylethoxy, pyrrolidylethoxy, N-pyrrolidylethoxy, oxo, ethylcarboxy, carboxymethyl-ethyl ester, pyridylmethyl, 4-pyridylmethoxy, 2-pyridylmethyl, and —COO—CH₂—CH₃, with the proviso that when G is —N—, R³⁶ is —H; and

wherein R³⁸ and R³⁹ are such that they optionally join to form a ring system of the type selected from:

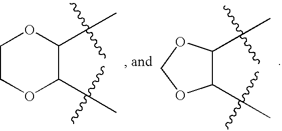

In another embodiment, the present invention comprises an aminocyanopyridine compound that provides an IC₅₀ of less than about 200 μM, in an in vitro assay of MK-2 inhibitory activity. Examples of such compounds comprise the compound shown in formula I, where:

R¹ is selected from the group consisting of —H, methyl, ethyl, —(CH₂)COOH, and phenyl;

R² is selected from the group consisting of —H, methyl, ethyl, amino, phenyl, methoxy, carboxy, hydroxyethylamino, propylamino, ethylamino, methylamino, methoxyethyl, ethoxyethylamino, aminoethylamino, benzylamino, dimethylaminoethylamino, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), acetylaminophenyl, methoxyphenylamino, carboxyphenyl, cyanophenyl, cyclopropyl, phenoxyphenyl, pyridyl, dihydroxybromophenyl, difluoromethoxyphenyl, trifluoromethylphenyl, trifluoromethylfluorophenyl, hydroxyphenyl,

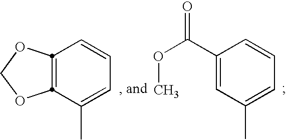

R³ is selected from the group consisting of —H, methyl, ethyl, propyl, isopropyl, cyano, and aminomethyl, except that when R² is pyrryl, R³ is other than cyano;

wherein the R² and R³ groups are such that they optionally join to form a ring system selected from:

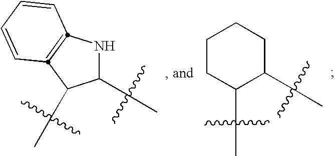

R⁴ is selected from the group consisting of —H, methyl, ethyl, propyl, hydroxy, furyl, indolyl, methylfuryl, methylimidazolyl, phenyl, hydroxyphenyl, carboxyphenyl, pyrazolyl, hydroxy, dihydroxyphenyl, methoxyphenyl, chlorophenyl, dichlorophenyl, dihydroxyborophenyl, thienyl, pyrryl, N-methylpyrryl, pyridyl, methylthio, methylsulfonylphenyl, carboethoxyphenyl, methoxy, carbamylphenyl, N-isoimidazoylphenyl, amino, hydroxynaphthyl, thiazoyl, carboxymethylphenyl, aminosulfonylphenyl, and

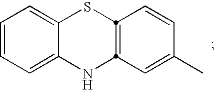

wherein the R³ and R⁴ groups are such that they can join to form a ring system selected from:

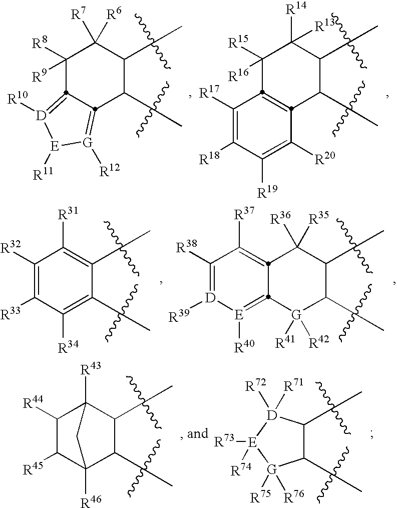

D, E and G are each independently selected from the group consisting of carbon, oxygen, sulfur, and nitrogen;

R⁵ is selected from the group consisting of —H, and C₁-C₅ alkyl, provided that at least one of R¹, R², R³, R⁴ and R⁵ is other than hydrogen;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰; R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁷¹, R⁷², R⁷³, R⁷⁴, R⁷⁵, and R⁷⁶ are each optionally present and are each independently selected from the group consisting of —H, methyl, ethyl, butyl, amino, nitro, hydroxy, methoxy, ethoxy, oxo, 2-propenoxy, carboxy, bromo, chloro, fluoro, trifluoromethyl, chloromethyl, hydroxymethyl, dicyanomethyl, hydroxyethoxy, ethoxyethoxy, —(CH₂)—O—(C₆H₄)—O—(CH₃), carboxymethoxy, isopropylcarboxymethoxy, methylamino, dimethylamino, aminoethoxy, diaminoethoxy, cyanomethoxymethyl, methoxymethyl, isopropoxymethyl, ethoxymethyl, —(CH₂)—O—(CF₂)—CHF₂, isobutoxymethyl, phenyl, morpholinylethoxy, pyrrolidylethoxy, N-pyrrolidylethoxy, and pyridylmethyl, with the proviso that when G is —N—, R³⁶ is —H; and

wherein R³⁸ and R³⁹ are such that they optionally join to form a ring system of the type selected from:

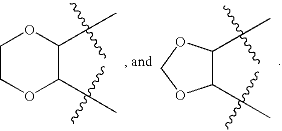

In another embodiment, the present invention comprises an aminocyanopyridine compound that provides an IC₅₀ of less than about 100 μM, in an in vitro assay of MK-2 inhibitory activity. Examples of such compounds comprise the compound shown in formula I, where:

R¹ is selected from the group consisting of —H, methyl, and ethyl;

R² is selected from the group consisting of —H, methyl, amino, phenyl, methoxy, hydroxyethylamino, propylamino, ethylamino, methylamino, methoxyethyl, ethoxyethylamino, aminoethylamino, benzylamino, dimethylaminoethylamino, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), acetylaminophenyl, methoxyphenylamino, carboxyphenyl, cyanophenyl, cyclopropyl, phenoxyphenyl, pyridyl, dihydroxybromophenyl, difluoromethoxyphenyl, and

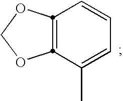

R³ is selected from the group consisting of —H, methyl, ethyl, propyl, isopropyl, and cyano, except that when R² is pyrryl, R³ is other than cyano;

wherein the R² and R³ groups are such that they optionally join to form a ring system selected from:

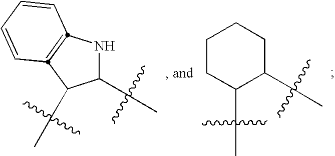

R⁴ is selected from the group consisting of —H, methyl, ethyl, propyl, hydroxy, furyl, indolyl, methylfuryl, methylimidazolyl, phenyl, hydroxyphenyl, carboxyphenyl, pyrazolyl, hydroxy, dihydroxyphenyl, methoxyphenyl, chlorophenyl, dichlorophenyl, dihydroxyborophenyl, thienyl, pyrryl, N-methylpyrryl, pyridyl, methylthio, methylsulfonylphenyl, carboethoxyphenyl, methoxy, carbamylphenyl, amino, and aminosulfonylphenyl;

wherein the R³ and R⁴ groups are such that they optionally join to form a ring system selected from:

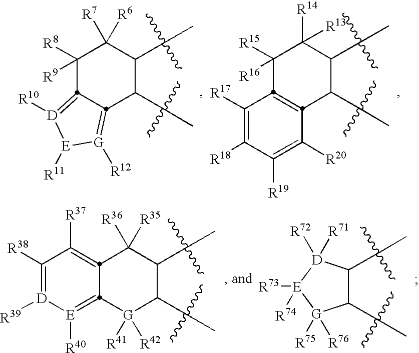

D, E and G are each independently selected from the group consisting of carbon, oxygen, sulfur, and nitrogen;

R⁵ is —H, provided that at least one of R¹, R², R³, R⁴, and R⁵ is other than hydrogen;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R³⁵, R³⁶, R³⁷, R³⁸, R³⁹, R⁴⁰, R⁴¹, R⁴², R⁷¹, R⁷², R⁷³, R⁷⁴, R⁷⁵, and R⁷⁶ are each optionally present and are each independently selected from the group consisting of —H, methyl, ethyl, butyl, amino, nitro, hydroxy, methoxy, ethoxy, oxo, 2-propenoxy, carboxy, bromo, fluoro, trifluoromethyl, chloromethyl, dicyanomethyl, hydroxyethoxy, ethoxyethoxy, —(CH₂)—O—(C₆H₄)—O—(CH₃), carboxymethoxy, isopropylcarboxymethoxy, methylamino, dimethylamino, aminoethoxy, diaminoethoxy, phenyl, morpholinylethoxy, pyrrolidylethoxy, N-pyrrolidylethoxy, and pyridylmethyl, with the proviso that when G is —N—, R³⁶ is —H; and

wherein R³⁸ and R³⁹ are such that they can join to form a ring system consisting of:

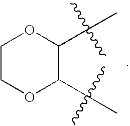

In another embodiment, the present invention comprises an aminocyanopyridine compound that provides an IC₅₀ of less than about 50 μM, in an in vitro assay of MK-2 inhibitory activity. Examples of such compounds comprise the compound shown in formula I, where:

R¹ is selected from the group consisting of —H, methyl, and ethyl;

R² is selected from the group consisting of —H, methyl, amino, phenyl, methoxy, hydroxyethylamino, propylamino, ethylamino, methylamino, methoxyethyl, ethoxyethylamino, aminoethylamino, benzylamino, dimethylaminoethylamino, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), acetylaminophenyl, methoxyphenylamino, carboxyphenyl, and

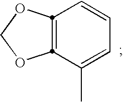

R³is selected from the group consisting of —H, methyl, ethyl, propyl, and isopropyl;

wherein the R² and R³ groups are optionally such that they join to form:

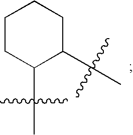

R⁴ is selected from the group consisting of —H, methyl, ethyl, propyl, furyl, indolyl, methylfuryl, methylimidazolyl, phenyl, hydroxyphenyl, carboxyphenyl, pyrazolyl, hydroxy, dihydroxyphenyl, methoxyphenyl, chlorophenyl, dichlorophenyl, dihydroxyborophenyl, thienyl, pyrryl, N-methylpyrryl, pyridyl, methylthio, methylsulfonylphenyl, carboethoxyphenyl, and aminosulfonylphenyl;

wherein the R³ and R⁴ groups are such that they optionally join to form a ring system selected from:

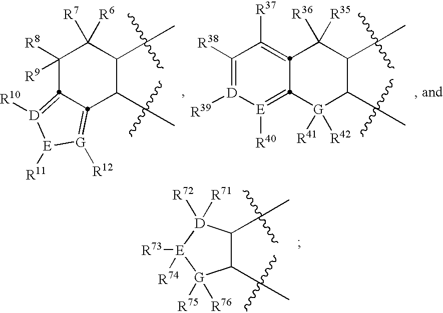

D, E and G are each independently selected from the group consisting of carbon, oxygen, sulfur, and nitrogen;

R⁵ is —H, provided that at least one of R¹, R², R³, R⁴ and R⁵ is other than hydrogen;

R⁶, R⁷, R⁸, R⁹, R¹⁰, R¹¹, R¹², R³⁵, R³⁶, R³⁷, R³⁸, R³⁹, R⁴⁰, R⁴¹, R⁴², R⁷¹, R⁷², R⁷³, R⁷⁴, R⁷⁵, and R⁷⁶ are each optionally present and are each independently selected from the group consisting of —H, methyl, ethyl, butyl, amino, nitro, hydroxy, methoxy, ethoxy, oxo, 2-propenoxy, carboxy, bromo, fluoro, trifluoromethyl, chloromethyl, dicyanomethyl, hydroxyethoxy, ethoxyethoxy, carboxymethoxy, isopropylcarboxymethoxy, methylamino, dimethylamino, aminoethoxy, diaminoethoxy, morpholinylethoxy, pyrrolidylethoxy, N-pyrrolidylethoxy, and pyridylmethyl, with the proviso that when G is —N—, R³⁶ is —H; and

wherein R³⁸ and R³⁹ are such that they optionally join to form a ring system consisting of:

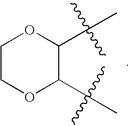

In another embodiment, the present invention comprises an aminocyanopyridine compound that provides an IC₅₀ of less than about 20 μM, in an in vitro assay of MK-2 inhibitory activity. Examples of such compounds comprise the compound shown in formula I, where:

R¹ is —H;

R² is selected from the group consisting of amino, phenyl, fluorophenyl, difluorophenyl, furyl, carbamylpyrryl, methyl-1,3-isodiazoyl, 1,3-isodiazoyl, 1,3,4-triazoyl, methoxyphenyl, acetylaminophenyl, methoxyphenylamino, and carboxyphenyl;

R³ is selected from the group consisting of —H, methyl, ethyl, and propyl;

R⁴ is selected from the group consisting of methyl, ethyl, propyl, furyl, phenyl, hydroxyphenyl, carboxyphenyl, pyrazolyl, hydroxy, dihydroxyphenyl, methoxyphenyl, chlorophenyl, dihydroxyborophenyl, and aminosulfonylphenyl;

wherein the R³ and R⁴ groups are such that they optionally join to form a ring system selected from:

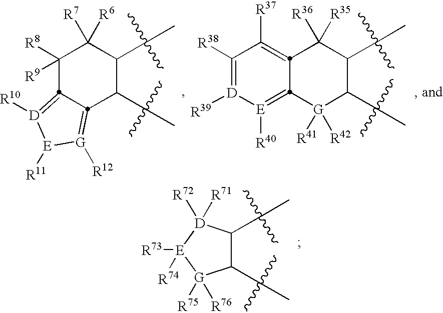

D, E and G are each independently selected from the group consisting of carbon, oxygen, sulfur, and nitrogen;

R⁵ is —H, provided that at least one of R¹, R², R³, R⁴ and R⁵ is other than hydrogen;

R⁶, R⁷, R⁸, R⁹, R¹⁰, R¹¹, R¹², R³⁵, R³⁶, R³⁷, R³⁸, R³⁹, R⁴⁰, R⁴¹, R⁴², R⁷¹, R⁷², R⁷³, R⁷⁴, R⁷⁵, and R⁷⁶ are each optionally present and are each independently selected from the group consisting of —H, amino, nitro, hydroxy, methoxy, ethoxy, oxo, 2-propenoxy, carboxy, bromo, fluoro, trifluoromethyl, chloromethyl, dicyanomethyl, hydroxyethoxy, ethoxyethoxy, carboxymethoxy, isopropylcarboxymethoxy, methylamino, dimethylamino, aminoethoxy, diaminoethoxy, morpholinylethoxy, pyrrolidylethoxy, and pyridylmethyl, with the proviso that when G is —N—, R³⁶ is —H; and

wherein R³⁸ and R³⁹ optionally are such that they optionally join to form:

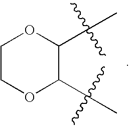

Examples of aminocyanopyridine MK-2 inhibitor compounds that can be used in the present method include, without limitation, the following:

2-amino-4-(2-fluorophenyl)-6,8-dihydro-5H-pyrazolo[3,4-h]quinoline-3-carbonitrile,

2-amino-4-(2-furyl)-6,7-dihydro-5H-pyrazolo[3,4-h]quinoline-3-carbonitrile,

2-amino-4-(2,3-difluorophenyl)-6,7-dihydro-5H-pyrazolo[3,4-h]quinoline-3-carbonitrile,

8-amino-6-(2-furyl)-4,5-dihydro-1H-pyrazolo[4,3-h]quinoline-7-carbonitrile,

2-amino-3-cyano-4-(2-furyl)-5,6-dihydrobenzo[h]quinoline-8-carboxylic acid,

4-[2-amino-3-cyano-6-(2-furyl)pyridin-4-yl]-1H-pyrrole-2-carboxamide,

2-amino-4-phenyl-6,8-dihydro-5H-pyrazolo[3,4-h]quinoline-3-carbonitrile,

2-amino-6-(2-furyl)-4-(1-methyl-1H-imidazol-4-yl)nicotinonitrile,

8-amino-6-(2-furyl)-4,5-dihydro-1H-pyrazolo[4,3-h]quinoline-7-carbonitrile,

2-amino-4-(2-furyl)-8-hydroxy-5,6-dihydrobenzo[h]quinoine-3-carbonitrile,

2-amino-4-(2,6-difluorophenyl)-6,7-dihydro-5H-pyrazolo[3,4-h]quinoline-3-carbonitrile,

2-amino-6-(4-hydroxyphenyl)-4-(1H-imidazol-5-yl)nicotinonitrile,

2-amino-4-(2-fluorophenyl)-6-(2-furyl)nicotinonitrile,

2-amino-4-(2-fluorophenyl)-6-(2-furyl)nicotinonitrile,

2-amino-4-(2-fluorophenyl)-5,6-dihydrobenzo[h]quinoline-3-carbonitrile,

4-[6-amino-5-cyano-4-(2-furyl)pyridin-2-yl]benzoic acid,

2-amino-6-(2-furyl)-4-(1H-imidazol-5-yl)nicotinonitrile,

2-amino-4-(2-furyl)-6-(1H-pyrazol-3-yl)nicotinonitrile,

2-amino-3-cyano-4-(4H- 1,2,4-triazol-3-yl)-5,6-dihydrobenzo[h]quinoline-8-carboxylic acid,

2-amino-6-(3-hydroxyphenyl)-4-(1H-imidazol-5-yl)nicotinonitrile,

2-amino-6-(2-furyl)-4-(1H-imidazol-4-yl)nicotinonitrile,

2-amino-4-(2,4-difluorophenyl)-6,7-dihydro-5H-pyrazolo[3,4-h]quinoline-3-carbonitrile,

4,6-diamino-2-(trifluoromethyl)-2,3-dihydrofuro[2,3-b]pyridine-5-carbonitrile,

2-amino-4-(2-furyl)-6,8-dihydro-5H-pyrrolo[3,4-h]quinoline-3-carbonitrile,

4-[6-amino-5-cyano-4-(2-fluorophenyl)pyridin-2-yl]benzoic acid,

2-amino-4-(2-furyl)-5,6-dihydro-1,8-phenanthroline-3-carbonitrile,

2-amino-6-(3,4-dihydroxyphenyl)-4-(2-fluorophenyl)nicotinonitrile,

2-amino-4-(1-methyl-1H-imidazol-4-yl)-6-phenylnicotinonitrile,

2-amino-4-(2-furyl)-6-(1H-pyrazol-3-yl)nicotinonitrile,

4-[6-amino-5-cyano-4-(1H-imidazol-5-yl)pyridin-2-yl]benzoic acid,

2-amino-4-(3-fluorophenyl)-6,8-dihydro-5H-pyrazolo[3,4-h]quinoline-3-carbonitrile,

2-amino-6-(3,4-dihydroxyphenyl)-4-(2-fluorophenyl)nicotinonitrile,

N-{4-[6-amino-5-cyano-4-(2-furyl)pyridin-2-yl]phenyl}methanesulfonamide,

2-amino-4-(2-furyl)-6,7-dihydro-5H-pyrrolo[2,3-h]quinoline-3-carbonitrile,

2-amino-4-(1H-imidazol-5-yl)-6-phenylnicotinonitrile,

2-amino-4-(2-furyl)-5,6-dihydrobenzo[h]quinoline-3-carbonitrile,

2-amino-4-(1H-imidazol-5-yl)-6-(4-methoxyphenyl)nicotinonitrile,

2-amino-6-(3-chlorophenyl)-4-(1H-imidazol-5-yl)nicotinonitrile,

2-amino-4-(2-furyl)-6-(1H-pyrazol-4-yl)nicotinonitrile,

2-amino-4-(4-methoxyphenyl)-6,7-dihydro-5H-pyrazolo[3,4-h]quinoline-3-carbonitrile,

2-amino-4-(2,5-difluorophenyl)-6,7-dihydro-5H-pyrazolo[3,4-h]quinoline-3-carbonitrile,

2-amino-4-(4-fluorophenyl)-6,8-dihydro-5H-pyrazolo[3,4-h]quinoline-3-carbonitrile,

2-amino-4-(4H-1,2,4-triazol-3-yl)-5,6-dihydrobenzo[h]quinoline-3-carbonitrile,

4,6-diamino-2-(chloromethyl)-2,3-dihydrofuro[2,3-b]pyridine-5-carbonitrile,

2-amino-4-(1H-imidazol-4-yl)-6-phenylnicotinonitrile,

4-[6-amino-5-cyano-4-(2-furyl)pyridin-2-yl]benzenesulfonamide,

4-[6-amino-5-cyano-4-(2-furyl)pyridin-2-yl]phenylboronic acid,

2-amino-6-(4-methoxyphenyl)-4-(4H-1,2,4-triazol-3-yl)nicotinonitrile,

2-amino-4-(2-fluorophenyl)-6-(3-furyl)nicotinonitrile,

2-amino-6-(2-furyl)-4-(methylthio)nicotinonitrile,

2-amino-4-(2-fluorophenyl)-6-(3-hydroxyphenyl)nicotinonitrile,

8-amino-6-(2-furyl)-4,5-dihydro-2H-pyrazolo[4,3-h]quinoline-7-carbonitrile,

2-amino-4-(2-bromophenyl)-6-(2-furyl)nicotinonitrile,

2-amino-4-(2-fluorophenyl)-6-(4-hydroxyphenyl)nicotinonitrile,

2-amino-4-phenyl-6-thien-2-ylnicotinonitrile,

2-amino-4-(3-methoxyphenyl)-6,7-dihydro-5H-pyrazolo[3,4-h]quinoline-3-carbonitrile,

2-amino-4-(2-furyl)-7-methyl-6,7-dihydro-5H-pyrazolo[3,4-h]quinoline-3-carbonitrile,

2-amino-4-(2-fluorophenyl)-6-(1H-pyrrol-2-yl)nicotinonitrile,

2-amino-4-(2-furyl)-5-methyl-6,8-dihydro-5H-pyrazolo[3,4-h]quinoline-3-carbonitrile,

2-amino-4-(2-furyl)-6-(1-methyl-1H-pyrrol-3-yl)nicotinonitrile,

3-amino-5,6,7,8-tetrahydroisoquinoline-4-carbonitrile,

N-[4-(2-amino-3-cyano-6,7-dihydro-5H-pyrazolo[3,4-h]quinolin-4-yl)phenyl]acetamide,

6-amino-4-[(4-methoxyphenyl)amino]-2-(trifluoromethyl)-2,3-dihydrofuro[2,3-b]pyridine-5-carbonitrile,

4-[6-amino-5-cyano-4-(2-furyl)pyridin-2-yl]- N-(tert-butyl)benzenesulfonamide,

4,6-diamino-2-ethyl-2,3-dihydrofuro[2,3-b]pyridine-5-carbonitrile,

6-amino-4-(2-furyl)-2,4′-bipyridine-5-carbonitrile,

2,4-diamino-6-(methylthio)nicotinonitrile,

3-(2-amino-3-cyano-6,7-dihydro-5H-pyrazolo[3,4-h]quinolin-4-yl)benzoic acid,

2-amino-6-(4-chlorophenyl)-4-(1H-imidazol-5-yl)nicotinonitrile,

2-amino-4-(1,3-benzodioxol-4-yl)-6,7-dihydro-5H-pyrazolo[3,4-h]quinoline-3-carbonitrile,

4,6-diamino-2-methyl-2,3-dihydrofuro[2,3-b]pyridine-5-carbonitrile,

2-amino-4-(1H-imidazol-5-yl)-6-[4-(methylsulfonyl)phenyl]nicotinonitrile,

2,4-diaminoquinoline-3-carbonitrile,

2,8-diamino-4-(2-furyl)-5,6-dihydrobenzo[h]quinoline-3-carbonitrile,

2-amino-4,6-di(2-furyl)nicotinonitrile,

4,6-diamino-2-butyl-2,3-dihydrofuro[2,3-b]pyridine-5-carbonitrile,

ethyl 4-[6-amino-5-cyano-4-(1H-imidazol-5-yl)pyridin-2-yl]benzoate,

2,4-diamino-6-methoxynicotinonitrile,

2-amino-4-methylnicotinonitrile,

2-amino-4-(4-cyanophenyl)-6,7-dihydro-5H-pyrazolo[3,4-h]quinoline-3-carbonitrile,

2-amino-4-cyclopropyl-6-methylnicotinonitrile,

2-amino-4-(2-furyl)-6-(1-methyl-1H-pyrrol-2-yl)nicotinonitrile,

2-amino-4-(2-chlorophenyl)-6,7-dihydro-5H-pyrazolo[3,4-h]quinoline-3-carbonitrile,

2-amino-6-(2-furyl)-4-(4-phenoxyphenyl)nicotinonitrile,

2-amino-4-pyridin-3-yl-6,8-dihydro-5H-pyrazolo[3,4-h]quinoline-3-carbonitrile,

2-amino-6-{[2-(4-chlorophenyl)-2-oxoethyl]thio}-4-(2-furyl)pyridine-3,5-dicarb onitrile,

4-[2-amino-3-cyano-6-(2-furyl)pyridin-4-yl]phenylboronic acid,

2-amino-6-(3-chlorophenyl)-4-(1H-imidazol-4-yl)nicotinonitrile,

4-(6-amino-5-cyano-4-phenylpyridin-2-yl)-N-(tert-butyl)benzenesulfonamide,

2-amino-4-methoxynicotinonitrile,

4-[2-amino-3-cyano-6-(2-furyl)pyridin-4-yl]benzoic acid,

4,6-diamino-2-[(4-methoxyphenoxy)methyl]-2,3-dihydrofuro[2,3-b]pyridine-5-carbonitrile,

2-amino-4-(2-fluorophenyl)-6-(4-methoxyphenyl)nicotinonitrile,

4-[6-amino-5-cyano-4-(2-fluorophenyl)pyridin-2-yl]-N-(tert-butyl)benzenesulfonamide,

(2,4-diamino-3-cyano-5H-chromeno[2,3-b]pyridin-9-yl)oxy]acetic acid,

3-Pyridinecarbonitrile; 2-Amino-4-Methylm

2-amino-6-(2-furyl)nicotinonitrile,

2-amino-4-(2-furyl)-6-(3-hydroxyphenyl)nicotinonitrile,

4-[6-amino-5-cyano-4-(2-furyl)pyridin-2-yl]benzamide,

2-amino-4-(2-furyl)-7-hydroxy-5,6-dihydrobenzo[h]quinoline-3-carbonitrile,

2-amino-4-(2-furyl)-6-(1H-indol-3-yl)nicotinonitrile,

2-amino-4-pyridin-4-yl-6,8-dihydro-5H-pyrazolo[3,4-h]quinoline-3-carbonitrile,

2-amino-4-(3-fluorophenyl)-6-(4-hydroxyphenyl)nicotinonitrile,

2-amino-4-[2-(difluoromethoxy)phenyl]-6,7-dihydro-5H-pyrazolo[3,4-h]quinoline-3-carbonitrile,

2-amino-4-(2-furyl)-6-thien-3-ylnicotinonitrile,

2-amino-4-(3-fluorophenyl)-6-(4-methoxyphenyl)nicotinonitrile,

2-[2-amino-3-cyano-6-(2-furyl)pyridin-4-yl]phenylboronic acid,

2,4-diamino-6-propylpyridine-3,5-dicarbonitrile,

4,6-diamino-2-[(prop-2-ynyloxy)methyl]-2 ,3-dihydrofuro[2,3-b]pyridine-5-carbonitrile,

4,6-diamino-2-(hydroxymethyl)-2,3-dihydrofuro[2,3-b]pyridine-5-carbonitrile,

2-amino-6-(2-furyl)-4-[4-(trifluoromethyl)phenyl]nicotinonitrile,

5-amino-7-methylthieno[3,2-b]pyridine-6-carbonitrile,

2-amino-4-(2-furyl)-5,5-dimethyl-6,8-dihydro-5H-pyrazolo[3,4-h]quinoline-3-carbonitrile,

N-[3-cyano-4-(2-fluorophenyl)-6-(2-furyl)pyridin-2-yl]glycine,

2-[(allyloxy)methyl]-4,6-diamino-2,3-dihydrofuro[2 ,3-b]pyridine-5-carbonitrile,

2-amino-4-(2-furyl)-6-methyl-5,6-dihydrobenzo[h]quinoline-3-carbonitrile,

4,6-diamino-2-(methoxymethyl)-2,3-dihydrofuro[2,3-b]pyridine-5-carbonitrile,

2-amino-4-(2-furyl)-6-(1H-indol-3-yl)nicotinonitrile,

2-amino-4-(2-furyl)-6-[4-(1H-imidazol-1-yl)phenyl]nicotinonitrile,

2-amino-4-(2-furyl)-6-(4-hydroxyphenyl)nicotinonitrile,

2-amino-4-(2-furyl)-5,6,7,8-tetrahydro-5,8-methanoquinoline-3-carbonitrile,

4,6-diamino-2-(isopropoxymethyl)-2,3-dihydrofuro[2,3-b]pyridine-5-carbonitrile,

3-[6-amino-5-cyano-4-(2-furyl)pyridin-2-yl]phenylboronic acid,

4,6-diamino-2-(ethoxymethyl)-2,3-dihydrofuro[2,3-b]pyridine-5-carbonitrile,

2-amino-4-(4-bromophenyl)-6-(2-furyl)nicotinonitrile,

4,6-diamino-2-[(1,1,2,2-tetrafluoroethoxy)methyl]-2,3-dihydrofuro[2,3-b]pyridine-5-carbonitrile,

2-amino-4-[2-fluoro-4-(trifluoromethyl)phenyl]-6-(2-furyl)nicotinonitrile,

2-amino-4-(2-methoxyphenyl)-6,8-dihydro-5H-pyrazolo[3,4-h]quinoline-3-carbonitrile,

2-amino-4-(2-fluorophenyl)-5-methyl-6,8-dihydro-5H-pyrazolo[3,4-h]quinoline-3-carbonitrile,

3,6-diamino-4-ethyl-1H-pyrazolo[3,4-b]pyridine-5-carbonitrile,

6-amino-4-(2-furyl)-2,2′-bipyridine-5-carbonitrile,

2-amino-4-(2-furyl)-6-(8-hydroxy-1-naphthyl)nicotinonitrile,

4-(2-amino-3-cyano-6,7-dihydro-5H-pyrazolo[3,4-h]quinolin-4-yl)benzoic acid,

2-amino-6-(3,4-dichlorophenyl)-4-(2-furyl)nicotinonitrile,

2-amino-4-(2-furyl)-6-(10H-phenothiazin-2-yl)nicotinonitrile,

sodium 2-amino-3-cyano-4-quinolinecarboxylate,

2-anilino-4-(2-fluorophenyl)-6-(2-furyl)nicotinonitrile,

2-amino-4-(3-fluorophenyl)-6-(2-furyl)nicotinonitrile,

2-amino-4-(4-fluorophenyl)-6-(2-furyl)nicotinonitrile,

4,6-diamino-2-(tert-butoxymethyl)-2,3-dihydrofuro[2,3-b]pyridine-5-carbonitrile,

2-amino-4-(2-furyl)-6-(1,3-thiazol-2-yl)nicotinonitrile,

4-(2-fluorophenyl)-6-(2-furyl)-2-piperidin-1-ylnicotinonitrile,

2-amino-6-(4-chlorophenyl)-4-(2-furyl)nicotinonitrile,

2-amino-6-(4-hydroxyphenyl)-4-(2-methoxyphenyl)nicotinonitrile,

2-amino-6-(2-furyl)-4-(2-hydroxyphenyl)nicotinonitrile,

methyl 3-(2-amino-3-cyano-6,7-dihydro-5H-pyrazolo[3,4-h]quinolin-4-yl)benzoate,

2-amino-4-(2-chlorophenyl)-6-(5-methyl-2-furyl)nicotinonitrile,

3,6-diamino-2-benzoylthieno[2,3-b]pyridine-5-carbonitrile,

methyl 4-[6-amino-5-cyano-4-(2-furyl)pyridin-2-yl]benzoate,

2-aminonicotinonitrile,

2-amino-4-(2-furyl)-8-{[2-(trimethylsilyl)ethoxy]methyl}-6,8-dihydro-5H-pyrazolo[3,4-h]quinoline-3-carbonitrile,

3-amino-5H-pyrido[4,3-b]indole-4-carbonitrile,

2-(2-amino-3-cyano-6,7-dihydro-5H-pyrazolo[3,4-h]quinolin-4-yl)benzoic acid,

2-amino-6-(4-methoxyphenyl)-4-phenylnicotinonitrile,

2-amino-4-(2-furyl)-5,6,7,8-tetrahydroquinoline-3-carbonitrile,

2-amino-4-(2-furyl)-6-isobutylnicotinonitrile,

2-amino-6-benzyl-4-(2-furyl)nicotinonitrile,

2-amino-4-(2-furyl)-6-methyl-5-phenylnicotinonitrile,

2-amino-4-(2-furyl)-6-[4-(trifluoromethoxy)phenyl]nicotinonitrile,

2-amino-4-(2-furyl)-6-propyl-5,6,7,8-tetrahydro-1,6-naphthyridine-3-carbonitrile,

2-amino-4-(2-furyl)benzo[h]quinoline-3-carbonitrile,

2-amino-6-(4-methoxyphenyl)-4-thien-2-ylnicotinonitrile,

2-amino-4-(2-fluorophenyl)-6-tetrahydrofuran-2-ylnicotinonitrile,

ethyl 6-amino-5-cyano-4-(2-furyl)pyridine-2-carboxylate,

2-amino-4-(2-furyl)-9-methoxy-5,6-dihydrobenzo[h]quinoline-3-carbonitrile,

2-amino-4-(2-furyl)-8-methoxy-5,6-dihydrobenzo[h]quinoline-3-carbonitrile,

2-amino-4-(2-furyl)-8,9-dimethoxy-5,6-dihydrobenzo[h]quinoline-3-carbonitrile,

2-amino-4-(2-furyl)-7-methoxy-5,6-dihydrobenzo[h]quinoline-3-carbonitrile,

2-amino-4-(2-furyl)-7,9-dimethyl-5,6-dihydrobenzo[h]quinoline-3-carbonitrile,

ethyl 4-[6-amino-5-cyano-4-(2-furyl)pyridin-2-yl]benzoate,

2-amino-6-(3-bromophenyl)-4-(2-furyl)nicotinonitrile,

2-amino-4-(2-furyl)-6-[4-(trifluoromethyl)phenyl]nicotinonitrile,

2-amino-4-(2-furyl)-6-[3-(trifluoromethyl)phenyl]nicotinonitrile,

2-amino-4-(2-furyl)-6-[4-(methylsulfonyl)phenyl]nicotinonitrile,

4,6-diamino-2-(phenoxymethyl)-2,3-dihydrofuro[2,3-b]pyridine-5-carbonitrile,

4,6-diamino-3-phenyl-2,3-dihydrofuro[2,3-b]pyridine-5-carbonitrile,

4,6-diamino-3-vinyl-2,3-dihydrofuro[2,3-b]pyridine-5-carbonitrile,

2-amino-4-(2-fluorophenyl)-5-methyl-6,8-dihydro-5H-pyrazolo[3,4-h]quinoline-3-carbonitrile,

3-amino-1-methyl-5,6,7,8-tetrahydroisoquinoline-4-carbonitrile,

2-amino-4-(2-fluorophenyl)-5,5-dimethyl-6,8-dihydro-5H-pyrazolo[3,4-h]quinoline-3-carbonitrile,

2-amino-4-(2-fluorophenyl)-6-(3-hydroxyphenyl)nicotinonitrile,

2-amino-4-[2-(difluoromethoxy)phenyl]-6,7-dihydro-5H-pyrazolo[3,4-h]quinoline-3-carbonitrile,

2-(benzylamino)-4-(2-fluorophenyl)-6-(2-furyl)nicotinonitrile,

2-amino-4-(2-furyl)-6,7-dihydro-5H-benzo[6,7]cyclohepta[1,2-b]pyridine-3-carbonitrile,

2-amino-4-(2-furyl)-5H-indeno[1,2-b]pyridine-3-carbonitrile,

3-amino-1-methyl-5,6,7,8-tetrahydroisoquinoline-4-carbonitrile,

2-amino-4-(2-fluorophenyl)-6-(3-hydroxyphenyl)nicotinonitrile,

2-amino-4-(2-thienyl)-5,6,7,8-tetrahydro-3-quinolinecarbonitrile,

2-amino-4-(3-fluorophenyl)-5,6,7,8-tetrahydro-3-quinolinecarbonitrile,

2-(1-piperidinyl)-6-(2-thienyl)-4-(trifluoromethyl)nicotinonitrile,

2-(dimethylamino)-6-(2-thienyl)-4-(trifluoromethyl)nicotinonitrile,

3-Quinolinecarbonitrile,

2-amino-4-methyl- or 2-amino-4-methyl-3-quinolinecarbonitrile,

2-amino-4-(4-methoxyphenyl)-6-(2-thienyl)nicotinonitrile,

2-amino-6-cyclopropyl-4-(2-methoxyphenyl)nicotinonitrile,

2-amino-4-(2-fluorophenyl)-6-phenylnicotinonitrile,

(4bS,8aR)-2,4-diamino-4b,5,6,7,8,8a-hexahydro[1]benzofuro[2,3-b]pyridine-3-carbonitrile,

2-amino-4-(2-fluorophenyl)-5,5-dimethyl-6,8-dihydro-5H-pyrazolo[3,4-h]quinoline-3-carbonitrile,

2-amino-4-(2-furyl)-5-phenyl-6,8-dihydro-5H-pyrazolo[3,4-h]quinoline-3-carbonitrile,

3-amino-1,6-dimethyl-5,6,7,8-tetrahydro-2,6-naphthyridine-4-carbonitrile,

3-amino-1,7-dimethyl-5,6,7,8-tetrahydro-2,7-naphthyridine-4-carbonitrile,

2-amino-4-(2-fluorophenyl)-5-phenyl-6,8-dihyd ro-5H-pyrazolo[3,4-h]quinoline-3-carbonitrile,

2-amino-4-(2-fluorophenyl)-5-phenyl-6,8-dihydro-5H-pyrazolo[3,4-h]quinoline-3-carbonitrile,

4,6-diamino-2-(morpholin-4-ylmethyl)-2,3-dihydrofuro[2,3-b]pyridine-5-carbonitrile,

ethyl (4,6-diamino-5-cyano-2-oxo-2,3-dihydro-1H-pyrrolo[2,3-b]pyridin-1-yl)acetate,

2-amino-4-(2-methoxyphenyl)-6-(5-methyl-2-furyl)nicotinonitrile,

2-amino-6-methyl-4-(4-nitrophenyl)nicotinonitrile,

2-amino-4-(3,4-dimethoxyphenyl)-6-(5-methyl-2-furyl)nicotinonitrile,

2,4-diamino-6-[(4-methoxyphenyl)thio]nicotinonitrile,

4,6-diamino-2-(phenoxymethyl)-2,3-dihydrofuro[2,3-b]pyridine-5-carbonitrile,

4,6-diamino-3-phenyl-2,3-dihydrofuro[2,3-b]pyridine-5-carbonitrile,

4,6-diamino-2-[(2-methylphenoxy)methyl]-2,3-dihydrofuro[2,3-b]pyridine-5-carbonitrile,

2-amino-4-(2-furyl)-6-(4-methoxyphenyl)nicotinonitrile,

2-amino-4-(3-fluorophenyl)-5,6-dihydrobenzo[h]quinoline-3-carbonitrile,

2-amino-4-(4-methoxyphenyl)-6,7-dihydro-5H-cyclopenta[b]pyridine-3-carbonitrile,

2-amino-9-ethyl-9H-pyrido[2,3-b]indole-3-carbonitrile,

2-amino-6-isobutyl-4-(4-methylphenyl)nicotinonitrile,

1-(2-furyl)-3-[(3-hydroxypropyl)amino]-5,6,7,8-tetrahydroisoquinoline-4-carbonitrile,

2-azepan-1-yl-6-(4-fluorophenyl)-4-phenylnicotinonitrile,

2-amino-6-tert-butyl-4-(4-methylphenyl)nicotinonitrile,

2-amino-4-(4-bromophenyl)-6-methylnicotinonitrile,

2-amino-4-thien-2-yl-5,6,7,8,9,10-hexahydrocycloocta[b]pyridine-3-carbonitrile,

2-amino-4-(4-chlorophenyl)-6,7,8,9-tetrahydro-5H-cyclohepta[b]pyridine-3-carbonitrile,

2-(allylamino)-5-amino-7-(4-bromophenyl)thieno[3,2-b]pyridine-3,6-dicarbonitrile,

2-amino-4-pyridin-3-yl-5,6,7,8,9,10-hexahydrocycloocta[b]pyridine-3-carbonitrile,

2-amino-4-(4-bromophenyl)-6-tert-butylnicotinonitrile,

1-(2-furyl)-3-morpholin-4-yl-5,6,7,8-tetrahydroisoquinoline-4-carbonitrile,

2-amino-4-(4-methylphenyl)-6,7-dihydro-5H-cyclopenta[b]pyridine-3-carbonitrile,

2-amino-7,7-dimethyl-7,8-dihydro-5H-pyrano[4,3-b]pyridine-3-carbonitrile,

2-amino-6-isobutyl-4-(4-methoxyphenyl)nicotinonitrile,

4,6-diamino-2-oxo-1-phenyl-2,3-dihydro-1H-pyrrolo[2,3-b]pyridine-5-carbonitrile,

2-amino-4-(2-methoxyphenyl)-5,6-dimethylnicotinonitrile,

2-(dimethylamino)-4-(2-fluorophenyl)-6-(2-furyl)nicotinonitrile,

2-(dimethylamino)-4-(2-fluorophenyl)-6-(2-furyl)nicotinonitrile,

4-(2-fluorophenyl)-6-(2-furyl)-2-(methylamino)nicotinonitrile,

4-(2-fluorophenyl)-6-(2-furyl)-2-morpholin-4-ylnicotinonitrile,

tert-butyl N-[3-cyano-4-(2-fluorophenyl)-6-(2-furyl)pyridin-2-yl]glycinate,

2-(ethylamino)-4-(2-fluorophenyl)-6-(2-furyl)nicotinonitrile,

ethyl 4-[6-amino-5-cyano-4-(2-fluorophenyl)pyridin-2-yl]benzoate,

2-amino-6-(2-fluorophenyl)-4-(3-furyl)nicotinonitrile,

6-amino-4-(2-fluorophenyl)-2,2′-bipyridine-5-carbonitrile,

2-amino-4-(2-fluorophenyl)-6-thien-2-ylnicotinonitrile,

ethyl 6-amino-5-cyano-4-(2-fluorophenyl)pyridine-2-carboxylate,

2-amino-6-(2-furyl)-4-phenylnicotinonitrile,

ethyl 2-amino-3-cyano-4-(2-furyl)-5,6,7,8-tetrahydroquinoline-6-carboxylate,

2-amino-4-(2-furyl)-6-(4-hydroxyphenyl)-5-methylnicotinonitrile,

2-amino-4-(2-furyl)-6-(4-methoxyphenyl)-5-methylnicotinonitrile,

2-amino-6-(4-fluorophenyl)-4-(2-furyl)-5-methylnicotinonitrile,

2-amino-4-(2-furyl)-5,6-diphenylnicotinonitrile,

2-amino-4-(2-furyl)-5-methyl-6-phenylnicotinonitrile,

2-amino-6-(3,4-dimethylphenyl)-4-(2-furyl)nicotinonitrile,

2-amino-6-(4-fluorophenyl)-4-(2-furyl)nicotinonitrile,

2-amino-4-(3-fluorophenyl)-6-(3-hydroxyphenyl)nicotinonitrile,

6-amino-4-(3-fluorophenyl)-2,4′-bipyridine-5-carbonitrile,

6-amino-4-(2-fluorophenyl)-2,4′-bipyridine-5-carbonitrile,

2-amino-4-butyl-6-methylnicotinonitrile,

2-amino-6-methyl-4-propylnicotinonitrile,

2-amino-4-ethyl-6-methylnicotinonitrile, 2-amino-4,6-dimethylnicotinonitrile,

2-amino-4-[2-(hexyloxy)phenyl]-6,7-dihydro-5H-pyrazolo[3,4-h]quinoline-3-carbonitrile,

2-amino-4-[2-(beta-D-glucopyranosyloxy)phenyl]-6,7-dihydro-5H-pyrazolo[3,4-h]quinoline-3-carbonitrile,

4-[2-(allyloxy)phenyl]-2-amino-6,7-dihydro-5H-pyrazolo[3,4-h]quinoline-3-carbonitrile,

methyl [2-(2-amino-3-cyano-6,7-dihydro-5H-pyrazolo[3,4-h]quinolin-4-yl)phenoxy]acetate,

2-amino-4-(2-ethoxyphenyl)-6,7-dihydro-5H-pyrazolo[3,4-h]quinoline-3-carbonitrile,

ethyl 4-[2-amino-3-cyano-6-(2-furyl)pyridin-4-yl]-1H-pyrrole-2-carboxylate,

2-amino-6-methylnicotinonitrile,

2-amino-6-(4-cyanophenyl)-4-(2-furyl)nicotinonitrile,

2-amino-6-(4-fluorobenzyl)-4-(2-furyl)nicotinonitrile,

2-amino-5-(4-fluorophenyl)-4-(2-furyl)-6-methylnicotinonitrile,

2-amino-4-(2-furyl)-6-(4-methoxyphenyl)nicotinonitrile,

2-amino-4-(2-methylphenyl)-5,6,7,8-tetrahydroquinoline-3-carbonitrile,

2-amino-4-(4-methoxyphenyl)-5,6,7,8-tetrahydroquinoline-3-carbonitrile,

2-amino-4-phenyl-5,6,7,8-tetrahydroquinoline-3-carbonitrile,

2-amino-6-(4-methoxyphenyl)-4-(2-methylphenyl)nicotinonitrile,

2-amino-4,6-bis(4-methoxyphenyl)nicotinonitrile,

2-amino-4-(3-chlorophenyl)-6-(4-methoxyphenyl)nicotinonitrile,

2-amino-4-(2-chlorophenyl)-6-(4-methoxyphenyl)nicotinonitrile,

2-amino-4-(2-furyl)-5,6,7,8-tetrahydro-1,6-naphthyridine-3-carbonitrile,

2-amino-4-(2-furyl)-6-(4-methylphenyl)nicotinonitrile,

2-amino-4-(2-furyl)-6-phenylnicotinonitrile,

6-amino-4-(2-furyl)-2,3′-bipyridine-5-carbonitrile,

2-amino-6-(1,3-benzodioxol-5-yl)-4-(2-furyl)nicotinonitrile,

2-amino-4-isoquinolin-4-yl-6-(4-methoxyphenyl)nicotinonitrile,

2-amino-4-(1-benzothien-3-yl)-6-(4-methoxyphenyl)nicotinonitrile,

2-amino-6-(4-methoxyphenyl)-4-thien-3-ylnicotinonitrile,

2-amino-4-(3-furyl)-6-(4-methoxyphenyl)nicotinonitrile,

2-amino-6-(4-methoxyphenyl)-4-(1H-pyrrol-2-yl)nicotinonitrile,

2-amino-4-(2-furyl)-6-(1H-pyrrol-2-yl)nicotinonitrile,

2′-amino-6′-(4-methoxyphenyl)-3,4′-bipyridine-3′-carbonitrile,

2-amino-4-[2-(trifluoromethoxy)phenyl]-6,7-dihydro-5H-pyrazolo[3,4-h]quinoline-3-carbo nitrile,

2-amino-4-(2-furyl)-5H-thiochromeno[4,3-b]pyridine-3-carbonitrile,

2-amino-4-{4-[(2-cyanoethyl)(methyl)amino]phenyl}-6,7-dihydro-5H-pyrazolo[3,4-h]quinoline-3-carbonitrile,

2-amino-4-[2-(2-hydroxyethoxy)phenyl]-6,7-dihydro-5H-pyrazolo[3,4-h]quinoline-3-carbonitrile,

2-amino-4-(2-methylphenyl)-6,7-dihydro-5H-pyrazolo[3,4-h]quinoline-3-carbonitrile,

2-amino-4-[4-(dimethylamino)phenyl]-6,7-dihydro-5H-pyrazolo[3,4-h]quinoline-3-carbonit rile,

2-amino-4-(1H-indol-7-yl)-6,7-dihydro-5H-pyrazolo[3,4-h]quinoline-3-carbonitrile,

methyl 4-(2-amino-3-cyano-6,7-dihydro-5H-pyrazolo[3,4-h]quinolin-4-yl)benzoate,

methyl 2-(2-amino-3-cyano-6,7-dihydro-5H-pyrazolo[3,4-h]quinolin-4-yl)benzoate,

[2-(2-amino-3-cyano-6,7-dihydro-5H-pyrazolo[3,4-h]quinolin-4-yl)phenoxy]acetic acid,

2-amino-6-phenylnicotinonitrile,

2-amino-6-cyclohexylnicotinonitrile,

2-amino-4-(2-furyl)-6-(1-trityl-1H-pyrazol-4-yl)nicotinonitrile,

2-amino-4-(2-fluorophenyl)-6-(4-hydroxyphenyl)nicotinonitrile,

It should be understood that salts and prodrugs of the aminocyanopyridine compounds that are described herein, as well as isomeric forms, tautomers, racemic mixtures of the compounds, and the like, which have the same or similar activity as the compounds that are described, are to be considered to be included within the description of the compound.

A general method for the synthesis of the aminocyanopyridine MK-2 inhibiting compounds of the present invention can be found in Kambe, S. et al., Synthesis 5:366-368 (1980). Further details of the synthesis of these aminocyanopyridines are provided in the examples.

The MK-2 inhibiting activity of an aminocyanopyridine compound can be determined by any one of several methods that are well known to those having skill in the art of enzyme activity testing. One such method is described in detail in the general methods section of the examples. In addition, the efficacy of an aminocyanopyridine MK-2 inhibiting compound in therapeutic applications can be determined by testing for inhibition of TNFα production in cell culture and in animal model assays. In general, it is preferred that the aminocyanopyridine MK-2 inhibiting compounds of the present invention be capable of inhibiting the production and/or the release of TNFα in cell cultures and in animal models.

In another embodiment of the present invention, a pharmaceutical composition, which contains one or more of the aminocyanopyridine MK-2 inhibitors, can be formulated for the purpose of the prevention or treatment of a TNFα mediated disease or disorder. The pharmaceutical composition includes a aminocyanopyridine MK-2 inhibitor of the present invention and a pharmaceutically acceptable carrier.

In another embodiment, a kit can be produced that is suitable for use in the prevention or treatment of a TNFα mediated disease or disorder. The kit comprises a dosage form comprising an aminocyanopyridine MK-2 inhibitor in an amount which comprises a therapeutically effective amount.

As used herein, an “effective amount” means the dose or effective amount to be administered to a patient and the frequency of administration to the subject which is readily determined by one of ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used, the nature and severity of the illness to be treated, as well as the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of one of the present MK-2 inhibitors for use in therapy which will achieve the goal of improvement in the severity of pain and inflammation and the frequency of incidence, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp.1707-1711.

The frequency of dose will depend upon the half-life of the active components of the composition. If the active molecules have a short half life (e.g. from about 2 to 10 hours) it may be necessary to give one or more doses per day. Alternatively, if the active molecules have a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 100 mg dosage of an aminocyanopyridine MK-2 inhibitor taken once every two days would be expressed as a dosage rate of 50 mg/day. Similarly, the dosage rate of an ingredient where 50 mg is taken twice per day would be expressed as a dosage rate of 100 mg/day.

For purposes of calculation of dosage amounts, the weight of a normal adult human will be assumed to be 70 kg.

When the aminocyanopyridine MK-2 inhibitor is supplied along with a pharmaceutically acceptable carrier, the pharmaceutical compositions that are described above can be formed. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the present invention are the isomeric forms and tautomers and the pharmaceutically-acceptable salts of the aminocyanopyridine MK-2 inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group Ia) salts, alkaline earth metal (Group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trifluoroacetate, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The aminocyanopyridine compounds of the present invention are useful for, but not limited to, the prevention and treatment of diseases and disorders that are mediated by TNFα. For example, the aminocyanopyridine MK-2 inhibitors of the invention would be useful to treat arthritis, including, but not limited to, rheumatoid arthritis, spondyloarthopathies, gouty arthritis, osteoarthritis, systemic lupus erythematosus and juvenile arthritis. Such aminocyanopyridine MK-2 inhibitor compounds of the invention would be useful in the treatment of asthma, bronchitis, menstrual cramps, tendinitis, bursitis, connective tissue injuries or disorders, and skin related conditions such as psoriasis, eczema, burns and dermatitis.

The aminocyanopyridine MK-2 inhibitor compounds of the present invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Such aminocyanopyridine MK-2 inhibiting compounds would be useful in treating inflammation in diseases and conditions such as herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

The aminocyanopyridine MK-2 inhibitors would also be useful in the treatment of ophthalmic diseases, such as retinitis, retinopathies, conjunctivitis, uveitis, ocular photophobia, and of acute injury to the eye tissue. These compounds would also be useful in the treatment of pulmonary inflammation, such as that associated with viral infections and cystic fibrosis. The compounds would also be useful for the treatment of certain central nervous system disorders such as cortical dementias including Alzheimer's disease.

As used herein, the terms “TNFα mediated disease or disorder” are meant to include, without limitation, each of the symptoms or diseases that is mentioned above.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of pain and/or inflammation associated with, but not limited to, any of the diseases or disorders described herein. Besides being useful for human treatment, the present compounds are also useful for treatment of mammals, including horses, dogs, cats, rats, mice, sheep, pigs, etc.

The term “subject” for purposes of treatment includes any human or animal subject who is in need of the prevention of or treatment of any one of the TNFα mediated diseases or disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any human or animal subject, and preferably is a subject that is in need of prevention and/or treatment of a TNFα mediated disease or disorder. The subject may be a human subject who is at risk of obtaining a TNFα mediated disease or disorder, such as those described above. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

In particular, the pharmaceutical compositions of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the aminocyanopyridine MK-2 inhibitors in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing one or more of the present MK-2 inhibitors may be formulated with sweetening agents, for example glycerol, sorbitol or sucrose. Such formulations may also contain a demulcent, a preservative and flavoring and coloring agents.

The subject compositions can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-, or di-, glycerides. In addition, n-3 polyunsaturated fatty acids may find use in the preparation of injectables.

The subject compositions can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### General Information for Preparation Methods

Unless otherwise noted, reagents and solvents were used as received from commercial suppliers.

NMR Analysis

Proton nuclear magnetic resonance spectra were obtained on a Varian Unity Innova 400, a Varian Unity Innova 300 a Varian Unity 300, a Bruker AMX 500 or a Bruker AV-300 spectrometer. Chemical shifts are given in ppm (δ) and coupling constants, J, are reported in Hertz. Tetramethylsilane was used as an internal standard for proton spectra and the solvent peak was used as the reference peak for carbon spectra. Mass spectra were obtained on a Perkin Elmer Sciex 100 atmospheric pressure ionization (APCI) mass spectrometer, a Finnigan LCQ Duo LCMS ion trap electrospray ionization (ESI) mass spectrometer, a PerSeptive Biosystems Mariner TOF HPLC-MS (ESI), or a Waters ZQ mass spectrometer (ESI).

Determination of MK-2 IC₅₀

Recombinant MAPKAPK2 was phosphorylated at a concentration of 42-78 μM by incubation with 0.23 μM of active p38α in 50 mM HEPES, 0.1 mM EDTA, 10 mM magnesium acetate, and 0.25 mM ATP, pH 7.5 for one hour at 30° C.

The phosphorylation of HSP-peptide (KKKALSRQLSVAA) by MAPKAPK2 was measured using an anion exchange resin capture assay method. The reaction was carried out in 50 mM β-glycerolphosphate, 0.04% BSA, 10 mM magnesium acetate, 2% DMSO and 0.8 mM dithiotheritol, pH 7.5 in the presence of the HSP-peptide with 0.2 μCi[γ³³P]ATP and 0.03 mM ATP. The reaction was initiated by the addition of 15 nM MAPKAPK2 and was allowed to incubate at 30° C. for 30 min. The reaction was terminated and [γ³³P]ATP was removed from solution by the addition of 150 μl of AG 1×8 ion exchange resin in 900 mM sodium formate pH 3.0. A 50 μl aliquot of head volume was removed from the quenched reaction mixture and added to a 96-well plate, 150 μl of Microscint-40 (Packard) was added and the amount of phosphorylated-peptide was determined. Allow the Microscint to sit in the plates for 60 minutes prior to counting.

Compounds are evaluated as potential inhibitors of the MK2 kinase by measuring their effects on MK2 phosphorylation of the peptide substrate. Compounds may be screened initially at two concentrations prior to determination of IC₅₀ values. Screening results are expressed as percent inhibition at the concentrations of compound tested. For IC₅₀ value determinations, compounds are tested at six concentrations in ten-fold serial dilutions with each concentration tested in triplicate. Results are expressed as IC₅₀ values in micromolar. The assay is performed at a final concentration of 2% DMSO.

Preferred aminocyanopyridine MK-2 inhibiting compounds of the present invention provide IC₅₀ values for MK-2 inhibition of below 200 μM. One method that can be used for determining the MK-2 inhibition IC₅₀ value is that described just above. More preferred aminocyanopyridine MK-2 inhibiting compounds have the capability of providing MK-2 inhibition IC₅₀ values of below 100 μM, yet more preferred of below 50 μM, even more preferred of below 20 μM, yet more preferred of below 10 μM, and even more preferred of below 5 μM.

U937 Cell TNFα Release Assay

The human monocyte-like cell line, U937 (ATCC #CRL-1593.2), is cultured in RPMI1640 media with 10% heat-inactivated fetal calf serum (GIBCO), glutamine and pen/strep at 37° C. and 5% CO₂. Differentiation of U937 to monocytic/macrophage-like cells is induced by the addition of phorbol12-myristate 13-acetate (Sigma) at final concentration of 20 ng/ml to a culture of U937 cells at ˜0.5 million cells/ml and incubated for 24 hrs. The cells are centrifuged, washed with PBS and resuspended in fresh media without PMA and incubated for 24 hrs. Cells adherent to the culture flask are harvested by scraping, centrifugation, and resuspended in fresh media to 2 million cells/ml, and 0.2 ml is aliquoted to each of 96 wells in flat-bottom plate. Cells are then incubated for an additional 24 hrs to allow for recovery. The media is removed from the cells, and 0.1 ml of fresh media is added per well. 0.05 ml of serially diluted compound or control vehicle (Media with DMSO) is added to the cells. The final DMSO concentration does not exceed 1%. After 1 hr incubation, 0.05 ml of 400 ng/ml LPS (E Coli serotype 0111:B4, Sigma) in media is added for final concentration of 100 ng/ml. Cells are incubated at 37° C. for 4 hrs. After 4 hrs incubation, supernatants are harvest and assayed by ELISA for the presence of TNFα.

U937 Cell TNFα ELISA

ELISA plates (NUNC-Immuno™ Plate Maxisorb™ Surface) were coated with purified mouse monoclonal IgG1 anti-human TNFα antibody (R&D Systems #MAB610; 1.25 ug/ml in sodium bicarbonate pH 8.0, 0.1 ml/well) and incubated at 4° C. Coating solution was aspirated the following day and wells were blocked with 1 mg/ml gelatin in PBS (plus 1× thimerasol) for 2 days at 4° C. Prior to using, wells were washed 3× with wash buffer (PBS with 0.05% Tween). Cultured media samples were diluted in EIA buffer (5 mg/ml bovine γ-globulin, 1 mg/ml gelatin, 1 ml/l Tween-20, 1 mg/ml thimerasol in PBS), added to wells (0.1 ml/well) in triplicate and allowed to incubate for 1.5 hr at 37° C. in a humidified chamber. Plates were again washed and 0.1 ml/well of a mixture of rabbit anti-human TNFα polyclonal antibodies in EIA buffer (1:400 dilution of Sigma #T8300, and 1:400 dilution of Calbiochem #654250) was added for 1 hr at 37° C. Plates were washed as before and peroxidase-conjugated goat anti-rabbit IgG (H+L) antibody (Jackson lmmunoResearch #111-035-144, 1 ug/ml in EIA buffer, 0.1 ml/well) was added for 45 min. After final washing, plates were developed with peroxidase-ABTS solution (Kirkegaard/Perry #50-66-01, 0.1 ml/well). Enzymatic conversion of ABTS to colored product was measured after 5-30 minutes using a SpectroMax 340 spectrophotometer (Molecular Devices) at 405 nm. TNF levels were quantitated from a recombinant human TNFα (R&D Systems #210-TA-010) standard curve using a quadratic parameter fit generated by SoftMaxPRO software. ELISA sensitivity was approximately 30 pg TNF/ml. IC₅₀ values for compounds were generated using BioAssay Solver.

Preferred aminocyanopyridine MK-2 inhibiting compounds of the present invention provide TNFα release IC₅₀ values of below 200 μM in an in vitro cell assay. One method that can be used for determining TNFα release IC₅₀ in an in vitro cell assay is that described just above. More preferred aminocyanopyridine MK-2 inhibiting compounds have the capability of providing TNFα release IC₅₀ values of below 100 μM, yet more preferred of below 50 μM, even more preferred of below 20 μM, yet more preferred of below 10 μM, even more preferred of below 5 μM, and yet more preferred of below 1.

Lipopolysaccharide (LPS)-Induced TNFα Production

Adult male 225-250 gram Lewis rats (Harlan Sprague-Dawley) were used. Rats were fasted 18 hr prior to oral dosing, and allowed free access to water throughout the experiment. Each treatment group consisted of 5 animals.

Compounds were prepared as a suspension in a vehicle consisting of 0.5% methylcellulose, 0.025% Tween-20 in PBS. Compounds or vehicle were orally administered in a volume of 1 ml using an 18 gauge gavage needle. LPS (E. coli serotype 0111:B4, Lot #39H4103, Cat. # L-2630, Sigma) was administered 1-4 hr later by injection into the penile vein at a dose of 1 mg/kg in 0.5 ml sterile saline. Blood was collected in serum separator tubes via cardiac puncture 1.5 hr after LPS injection, a time point corresponding to maximal TNFα production. After clotting, serum was withdrawn and stored at −20° C. until assay by ELISA (described below).

Rat LPS TNFα ELISA

ELISA plates (NUNC-Immuno™ Plate Maxisorb™ Surface) were coated with 0.1 ml per well of an Protein G purified fraction of a 2.5 ug/ml of hamster anti-mouse/rat TNFα monoclonal antibody TN19.12 (2.5 ug/ml in PBS, 0.1 ml/well). The hybridoma cell line was kindly provided by Dr. Robert Schreiber, Washington University. Wells were blocked the following day with 1 mg/ml gelatin in PBS. Serum samples were diluted in a buffer consisting of 5 mg/ml bovine γ-globulin, 1 mg/ml gelatin, 1 ml/l Tween-20, 1 mg/ml thimerasol in PBS, and 0.1 ml of diluted serum was added wells in duplicate and allowed to incubate for 2 hr at 37° C. Plates were washed with PBS-Tween, and 0.1 ml per well of a 1:300 dilution of rabbit anti-mouse/rat TNFα antibody (BioSource International, Cat. #AMC3012) was added for 1.5 hr at 37° C. Plates were washed, and a 1:1000 fold dilution of peroxidase-conjugated donkey anti-rabbit IgG antibody (Jackson ImmunoResearch, Cat. #711-035-152) was added for 45 min. After washing, plates were developed with 0.1 ml of ABTS-peroxide solution (Kirkegaard/Perry, Cat. #50-66-01). Enzymatic conversion of ABTS to colored product was measured after ˜30 minutes using a SpectroMax 340 spectrophotometer (Molecular Devices Corp.) at 405 nm. TNF levels in serum were quantitated from a recombinant rat TNFα (BioSource International, Cat. #PRC₃₀₁₄.) standard curve using a quadratic parameter fit generated by SoftMaxPRO software. ELISA sensitivity was approximately 30 pg TNF/ml. Results are expressed in percent inhibition of the production of TNFα as compared to blood collected from control animals dosed only with vehicle.

Preferred aminocyanopyridine MK-2 inhibiting compounds of the present invention are capable of providing some degree of inhibition of TNFα in animals. That is, the degree of inhibition of TNFα in animals is over 0%. One method for determining the degree of inhibition of TNFα is the rat LPS assay that is described just above. More preferred aminocyanopyridine MK-2 inhibiting compounds have the capability of providing rat LPS TNFα inhibition values of at least about 25%, even more preferred of above 50%, yet more preferred of above 70%, and even more preferred of above 80%.

Synthesis of Aminocyanopyridine Compounds

A general method forthe synthesis of aminocyanopyridines can be found in Kambe, S. et al., “A simple method for the preparation of 2-amino-4-aryl-3-cyanopyridines by the condensation of malononitrile with aromatic aldehydes and alkyl ketones in the presence of ammonium acetate”, Synthesis 5:366-368 (1980). Further details of the synthesis of aminocyanopyridines of the present invention are provided below.

### EXAMPLE 1

This example illustrates the production of 2-amino-6-(3,4-dihydroxyphenyl)-4-(2-fluorophenyl)nicotinonitrile trifluoroacetate.

2-Fluorobenzaledhyde (5 mmol, 1.0 equiv., 530 82 L), 3,4-dihydroxyacetophenone (5 mmol, 1.0 equiv., 760 mg) malononitrile (5 mmol, 1.0 equiv., 290 μL) and ammonium acetate (7.5 mmol, 1.5 equiv., 578 mg) were combined in dichloroethane (10 mL) and heated to reflux for 4 hours. Dichloroethane was evaporated and the residue was purified by reverse phase chromatography. The product was isolated as an orange solid (145 mg, 8% yield). ¹H NMR (400 MHz, DMSO) δ 7.70 (d, 1H), 7.59-7.53 (m, 3H), 7.37 (d, 1H), 7.32 (t, 1H), 7.18 (s, 1H), 6.90 (d, 1H), 6.34 (bs, 1H) 3.21 (bs, 4H): m/z 322 (M+H).

### EXAMPLE 2

This example illustrates the production of 2-amino-4-(2-fluorophenyl)-6-(2-furyl)nicotinonitrile trifluoroacetate.

2-Fluorobenzaledhyde (2 mmol, 1.0 equiv., 210 μL), and malononitrile (2 mmol, 1.0 equiv., 126 μL) were combined in toluene (3 mL) and heated to 50° C. for 0.5 hours. 2-acetyl furan (2 mmol, 1.0 equiv., 146 mg) and ammonium acetate (3 mmol, 1.5 equiv., 230 mg) were added and the reaction stirred at 55° C. overnight. Amberlyst resin (1 g) was added and the reaction was diluted with dichloromethane. After shaking overnight, the resin was isolated by filtration and washed with dichloromethane and methanol. The resin was treated with 2M ammonia in methanol. After shaking overnight, the resin was removed by filtration and the filtrate concentrated under a stream of nitrogen. The residue was purified by reverse phase chromatography and the product was isolated as a brown solid (50 mg, 9%). ¹H NMR (300 MHz, DMSO) δ 7.78 (s, 1H), 7.65-7.75 (m, 2H), 7.43-7.35 (m, 2H), 7.22 (d, 1H), 7.14 (s, 1H), 6.67 (s, 1H) 6.48 (bs, 2H): m/z 280 (M+H).

### EXAMPLE 3

This example illustrates the production of 2-amino-6-(4-hydroxyphenyl)-4-(1H-imidazol-5-yl)nicotinonitrile trifluoroacetate.

Step 1: Production of 2-(1H-imidazol-5-ylmethylene)malononitrile.

1H-imidazole-5-carbaldehyde (20 mmol, 1.0 equiv., 1.92 g), and malononitrile (20 mmol, 1.0 equiv., 1.26 mL) were combined in trimethylorthoformate (30 mL) and triethylamine (7 mL). After stirring at room temperature overnight, the solvents were evaporated and the residue partitioned between 1M hydrochloric acid (HCl) and dichloromethane. The aqueous layer was neutralized with sodium bicarbonate and extracted with ethyl acetate (3×100 mL). The combined organic extracts were dried over magnesium sulfate (MgSO₄), filtered and evaporated to give the product as a yellow solid (2.58 g, 90%). ¹H NMR (400 MHz, Acetone) δ 12.11 (bs, 1H), 8.07 (s, 1H), 8.04 (s, 1H), 7.95 (s, 1H): m/z 143 (M−H).

Step 2: Production of 2-[(1-{[2-(trimethylsilyl)ethoxy]methyl}-1H-inidazol-5-yl)methylene)malononitrile;

2-(1H-imidazol-5-ylmethylene)malononitrile, (2 mmol, 1.0 equiv., 288 mg), prepared as described in Step 1, was added to a cool (0° C.) suspension of sodium hydride (60% in mineral oil, 1.1 equiv., 50 mg) in THF (15 mL). After 20 minutes, [2-(chloromethoxy)ethyl](trimethyl)silane (2.2 mmol, 1.1 equiv., 390 μL) was added and the solution warmed to room temperature overnight. The reaction was treated with water (5 mL) and concentrated the residue was extracted with ethyl acetate (25 mL) and the layers separated. Dried organic extract with MgSO₄, filtered and evaporated to give a brown solid. The product was purified by silica gel chromatography. The product was isolated as a yellow solid, (277 mg, 50%). ¹H NMR (400 MHz, CDCl₃) 7.98 (s, 1H), 7.76 (s, 1H), 5.34 (s, 2H) 3.52 (dd, 2H), 0.92 (dd, 2H), −0.01 (s, 9H): m/z 275 (M+H).

Step 3: Production of 2-amino-6-(4-hydroxyphenyl)-4-(1H-imidazol-5-yl)nicotinonitrile trifluoroacetate.

2-[(1-{[2-(trimethylsilyl)ethoxy]methyl}-1H-inidazol-5-yl)methylene)malononitrile (0.8 mmol, 1.0 equiv., 220 mg), prepared as described in Step 2, above, 4-hydroxyacetophenone (0.8 mmol, 1.0 equiv., 109 mg) and ammonium acetate (1.2 mmol, 1.5 equiv., 95 mg) were combined in toluene (3 mL) and benzene (1 mL) heated to 80° C. overnight. After cooling, Amberlyst resin (1 g) was added and the mixture heated to 50° C. overnight. The resin was isolated by filtration and washed with dichloromethane and methanol. The resin was treated with 2M ammonia in methanol. The resin was removed by filtration and the filtrate concentrated under a stream of nitrogen. The residue was purified by reverse phase chromatography and the product was isolated as a solid (25 mg, 11%). ¹H NMR (300 MHz, Acetone) δ 8.59 (s, 1H), 8.32 (s, 1H), 8.12 (d, 2H), 7.87 (s, 1H), 6.97 (d, 2H), 6.73 (bs, 1H): m/z 278 (M+H).

### EXAMPLE 4

This illustrates the production of 2-amino-6-(3-hydroxyphenyl)-4-(1H-imidazol-5-yl)nicotinonitrile trifluoroacetate.

2-amino-6-(3-hydroxyphenyl)-4-(1H-imidazol-5-yl)nicotinonitrile trifluoroacetate was prepared in the same manner as 2-amino-6-(4-hydroxyphenyl)-4-(1H-imidazol-5-yl)nicotinonitrile trifluoroacetate, as described in Example 3. The amount produced was 25 mg, at a yield of 11%. ¹H NMR (300 MHz, Acetone) δ 8.51 (s, 1H), 8.32 (s, 1H), 7.93 (s, 1H), 7.76 (t, 1H) 7.66 (d, 2H), 7.34 (t, 1H), 6.98 (dd, 1H), 6.59 (bs, 1H): m/z 278 (M+H). TNFα release assay IC50: 7.0 μM; Rat LPS assay: 41% inhibition of TNFα production at 20 mpk (IG).

### EXAMPLE 5

This illustrates the production of 2-amino-6-(2-furyl)-4-(1H-imidazol-5-yl)nicotinonitrile trifluoroacetate.

2-amino-6-(2-furyl)-4-(1H-imidazol-5-yl)nicotinonitrile trifluoroacetate was prepared in the same manner as 2-amino-6-(4-hydroxyphenyl)-4-(1H-imidazol-5-yl)nicotinonitrile trifluoroacetate, as described in Example 3. The amount produced was 20 mg, at a yield of 10%. ¹H NMR (300 MHz, Acetone) δ 8.40 (s, 1H), 8.29 (s, 1H), 7.81 (m, 2H), 7.27 (d, 1H), 6.70-6.68 (m, 2H): m/z 252 (M+H).

### EXAMPLE 6

This illustrates the production of the intermediate, 2-[1-(1-methyl-1H-imidazol-4-yl)ethylidene]malononitrile.

2-(1H-imidazol-5-ylmethylene)malononitrile (3.92 mmol, 1.0 equiv., 565 mg), prepared as described in Step 1 of Example 3, was dissolved in tetrahydrofuran (THF) and cooled to 0° C. Sodium hydride (60% in mineral oil, 1.1 equiv., 103 mg) as added followed by dimethylsulfate (4.31 mmol, 1.1 equiv., 410 μL). The solution warmed to room temperature overnight. The reaction was treated with water and extracted with ethyl acetate. The organic extract was dried with MgSO₄, filtered and evaporated to give a solid. The product was isolated as a white solid, (500 mg, 80%). ¹H NMR (300 MHz, Acetone) 8.01 (s, 2H), 7.85 (s, 1H), 3.92: m/z 159 (M+H).

### EXAMPLE 7

This illustrates the production of 2-amino-6-(2-furyl)-4-(1-methyl-1H-imidazol-4-yl)nicotinonitrile bis(trifluoroacetate).

2-[1-(1-methyl-1H-imidazol-4-yl)ethylidene]malononitrile (1.0 mmol, 1.0 equiv., 158 mg), 2-acetylfuran (1.0 mmol, 1.0 equiv., 100 μL) and ammonium acetate (1.5 mmol, 1.5 equiv., 115 mg) were combined in toluene (2 mL) and benzene (1 mL) heated to 70° C. overnight. After cooling, Amberlyst resin (1 g) was added and the mixture shaken overnight. The resin was isolated by filtration and washed with dichloromethane and methanol. The resin was treated with 2M ammonia in methanol. The resin was removed by filtration and the filtrate concentrated under a stream of nitrogen. The residue was purified by reverse phase chromatography and the product was isolated as a solid (35 mg, 13%). ¹H NMR (400 MHz, Acetone) δ 8.08 (s, 1H), 7.91 (s, 1H), 7.81 (s, 1H), 7.76 (s, 1H), 7.19 (d, 1H), 6.64 (d, 1H) 6.46 (bs, 2H), 3.94 (s, 3H): m/z 266 (M+H).

### EXAMPLE 8

This illustrates the production of 2-amino-4-(1-methyl-1H-imidazol-4-yl)-6-phenylnicotinonitrile bis(trifluoroacetate).

2-amino-4-(1-methyl-1H-imidazol-4-yl)-6-phenylnicotinonitrile bis(trifluoroacetate) was prepared in the same manner as 2-amino-6-(2-furyl)-4-(1-methyl-1H-imidazol-4-yl)nicotinonitrile bis(trifluoroacetate), as described in Example 7, with the production of 40 mg of solid material and with a yield of 13%. ¹H NMR (400 MHz, Acetone) δ 8.15 (bs, 4H), 7.91 (s, 1H), 7.48 (s, 3H), 4.00 (s, 3H): m/z 276 (M+H).

### EXAMPLES 9-58

This illustrates the production of aminocyanopyridine compounds of the present invention.

The compounds listed in the table below were prepared by the methods described in Kambe, S. et al., “A simple method for the preparation of 2-amino-4-aryl-3-cyanopyridines by the condensation of malononitrile with aromatic aldehydes and alkyl ketones in the presence of ammonium acetate”, Synthesis 5:366-368 (1980). NMR analysis was carried out for each compound and selected data is presented for each compound as shown in the table.

| Ex. No. | Compound name | m/z (M + H) |
|---|---|---|
| 9 | 4-[6-amino-5-cyano-4-(1H-imidazol-5-yl)pyridin-2-yl]benzoic | 306 |
| | acid hydrochloride | |
| 10 | 2-amino-6-(3,4-dihydroxyphenyl)-4-(2- | 322 |
| | fluorophenyl)nicotinonitrile | |
| 11 | 2-amino-4-(1H-imidazol-5-yl)-6-phenylnicotinonitrile | 262 |
| | trifluoroacetate | |
| 12 | 2-amino-4-(1H-imidazol-5-yl)-6-(4- | 292 |
| | methoxyphenyl)nicotinonitrile trifluoroacetate | |
| 13 | 8-ethoxy-2,4-bis(ethylamino)-5H-chromeno[2,3-b]pyridine-3- | 339 |
| | carbonitrile | |
| 14 | 2-amino-6-(3-chlorophenyl)-4-(1H-imidazol-5- | 296 |
| | yl)nicotinonitrile trifluoroacetate | |
| 15 | 4-[6-amino-5-cyano-4-(2-furyl)pyridin-2- | 341 |
| | yl]benzenesulfonamide trifluoroacetate | |
| 16 | 2-amino-4-(2-fluorophenyl)-6-(3-hydroxyphenyl)nicotinonitrile | 306 |
| | trifluoroacetate | |
| 17 | 2-amino-4-(2-bromophenyl)-6-(2-furyl)nicotinonitrile | 340 |
| | trifluoroacetate | |
| 18 | 2-amino-4-(2-fluorophenyl)-6-(4-hydroxyphenyl)nicotinonitrile | 306 |
| | trifluoroacetate | |
| 19 | 2-amino-6-(4-chlorophenyl)-4-(1H-imidazol-5- | 296 |
| | yl)nicotinonitrile trifluoroacetate | |
| 20 | 2-amino-4-(1H-imidazol-5-yl)-6-[4- | 340 |
| | (methylsulfonyl)phenyl]nicotinonitrile trifluoroacetate | |
| 21 | ethyl 4-[6-amino-5-cyano-4-(1H-imidazol-5-yl)pyridin-2- | 334 |
| | yl]benzoate trifluoroacetate | |
| 22 | 2-amino-4-cyclopropyl-6-methylnicotinonitrile trifluoroacetate | 174 |
| 23 | 2-amino-6-(2-furyl)-4-(4-phenoxyphenyl)nicotinonitrile | 354 |
| | trifluoroacetate | |
| 24 | 4-[2-amino-3-cyano-6-(2-furyl)pyridin-4-yl]phenylboronic acid | 306 |
| | trifluoroacetate | |
| 25 | 4-[2-amino-3-cyano-6-(2-furyl)pyridin-4-yl]benzoic acid | 306 |
| | trifluoroacetate | |
| 26 | 2-amino-4-(2-fluorophenyl)-6-(4- | 320 |
| | methoxyphenyl)nicotinonitrile trifluoroacetate | |
| 27 | 2-amino-4-(3-fluorophenyl)-6-(4-hydroxyphenyl)nicotinonitrile | 306 |
| | trifluoroacetate | |
| 28 | 2-amino-4-(3-fluorophenyl)-6-(4- | 320 |
| | methoxyphenyl)nicotinonitrile trifluoroacetate | |
| 29 | 2-[2-amino-3-cyano-6-(2-furyl)pyridin-4-yl]phenylboronic acid | 306 |
| | trifluoroacetate | |
| 30 | 2-amino-6-(2-furyl)-4-[4-(trifluoromethyl)phenyl]nicotinonitrile | 330 |
| | trifluoroacetate | |
| 31 | 2-amino-4-(4-bromophenyl)-6-(2-furyl)nicotinonitrile | 340 |
| | trifluoroacetate | |
| 32 | 2-amino-4-[2-fluoro-4-(trifluoromethyl)phenyl]-6-(2- | 348 |
| | furyl)nicotinonitrile trifluoroacetate | |
| 33 | 2-amino-4-(3-fluorophenyl)-6-(2-furyl)nicotinonitrile | 280 |
| | trifluoroacetate | |
| 34 | 2-amino-4-(4-fluorophenyl)-6-(2-furyl)nicotinonitrile | 280 |
| | trifluoroacetate | |
| 35 | 2-amino-6-(4-methoxyphenyl)-4-thien-3-ylnicotinonitrile | 308 |
| | trifluoroacetate | |
| 36 | 2-amino-4-(3-furyl)-6-(4-methoxyphenyl)nicotinonitrile | 292 |
| | trifluoroacetate | |
| 37 | 2-amino-6-(4-methoxyphenyl)-4-(1H-pyrrol-2-yl)nicotinonitrile | 291 |
| | trifluoroacetate | |
| 38 | 2-amino-6-(4-methoxyphenyl)-4-thien-2-ylnicotinonitrile | 308 |
| | trifluoroacetate | |
| 39 | 2-amino-4-(3-chlorophenyl)-6-(4- | 336 |
| | methoxyphenyl)nicotinonitrile trifluoroacetate | |
| 40 | 2-amino-4-(2-chlorophenyl)-6-(4- | 336 |
| | methoxyphenyl)nicotinonitrile trifluoroacetate | |
| 41 | 2′-amino-6′-(4-methoxyphenyl)-3,4′-bipyridine-3′-carbonitrile | 303 |
| | trifluoroacetate | |
| 42 | 2-amino-4-isoquinolin-4-yl-6-(4-methoxyphenyl)nicotinonitrile | 353 |
| | trifluoroacetate | |
| 43 | 2-amino-4-(1-benzothien-3-yl)-6-(4- | 358 |
| | methoxyphenyl)nicotinonitrile trifluoroacetate | |
| 44 | 2-amino-4-(2-furyl)-6-(4-methoxyphenyl)nicotinonitrile | 292 |
| | trifluoroacetate | |
| 45 | 2-amino-4-(2-methylphenyl)-5,6,7,8-tetrahydroquinoline-3- | 263 |
| | carbonitrile trifluoroacetate | |
| 46 | 2-amino-4-(4-methoxyphenyl)-5,6,7,8-tetrahydroquinoline-3- | 280 |
| | carbonitrile trifluoroacetate | |
| 47 | 2-amino-4-phenyl-5,6,7,8-tetrahydroquinoline-3-carbonitrile | 250 |
| 48 | 2-amino-6-(4-methoxyphenyl)-4-(2- | 316 |
| | methylphenyl)nicotinonitrile trifluoroacetate | |
| 49 | 2-amino-4,6-bis(4-methoxyphenyl)nicotinonitrile | 332 |
| | trifluoroacetate | |
| 50 | 2-amino-6-(4-methoxyphenyl)-4-phenylnicotinonitrile | 302 |
| | trifluoroacetate | |
| 51 | 2-amino-4-butyl-6-methylnicotinonitrile trifluoroacetate | 190 |
| 52 | 2-amino-6-methyl-4-propylnicotinonitrile trifluoroacetate | 176 |
| 53 | 2-amino-4-ethyl-6-methylnicotinonitrile trifluoroacetate | 162 |
| 54 | 2-amino-4,6-dimethylnicotinonitrile trifluoroacetate | 148 |
| 55 | 6-amino-4-(3-fluorophenyl)-2,4′-bipyridine-5-carbonitrile | 291 |
| | trifluoroacetate | |
| 56 | 2-amino-4-(3-fluorophenyl)-6-(3-hydroxyphenyl)nicotinonitrile | 306 |
| | trifluoroacetate | |
| 57 | 2-amino-4-(3-fluorophenyl)-6-(3-hydroxyphenyl)nicotinonitrile | 306 |
| | trifluoroacetate | |
| 58 | 6-amino-4-(2-fluorophenyl)-2,4′-bipyridine-5-carbonitrile | 291 |
| | trifluoroacetate | |

### EXAMPLE 59

This illustrates the production of 4-[2-amino-3-cyano-6-(2-furyl)pyridin-4-yl]-1H-pyrrole-2-carboxamide.

A mixture of malononitrile (20 mmol, 1.32 g), ethyl 4-formylpyrrole-2-carboxylate (20 mmol, 3.34 g), 2-acetylfuran (20 mmol, 2.2 g) and ammonium acetate (30 mmol, 2.32 g) in toluene (25 mL) was heated under reflux for 24 hours with azeotropic removal of water. After cooling to room temperature, the reaction mixture was evaporated under reduced pressure to dryness and the residue was stirred with ethanol (15 ml) for 4 hours. The resultant precipitate was collected by filtration, washed with aqueous ethanol and air-dried. Recrystallization of the solid from tetrahydrofuran gave a yellow-brown powder (2.25 g, 35% yield): ¹H NMR (400 MHZ, DMSO) δ 12.42 (s, 1H), 7.836 (s, 1H), 7.776 (d, 1H), 7.404 (d, 1H), 7.220 (s, 1H), 7.195 (d, 1H), 6.797 (s, 2H), 6.642(dd, 1H), 4.257 (q, 2H), 1.277 (t, 3H).

To a suspension of the above solid (5 mmol, 1.6 g) in ethanol (50 mL) was added aqueous sodium hydroxide(10% wt/volume, 15 mmol, 6 ml) and the mixture was warmed at 60° C. for 5 hours. The resultant solution was kept at room temperature overnight and then evaporated under reduced pressure. The residue was dissolved in warm water (50 ml), then acidified with 5% HCl solution to pH=3. The resultant precipitate was collected by filtration, washed with water and dried under vacuum to give a greylsh powder. To a solution of the above solid (1 mmol, 0.294 g) in dry dimethylformamide (12 ml) was added 1,1′-carbonyldiimidazole (1.2 mmol, 0.195 g) in one portion and the mixture was stirred at 50° C. for 2 hours. After cooling to room temperature, ammonia was bubbled into the reaction mixture for 30 minutes and then kept at room temperature for 48 hours. The mixture was evaporated in vacuo to dryness and the residue was stirred with water (10 ml). The resultant precipitate was collected by filtration, washed successively with water and ether and recrystallized from methanol to give the product as a gray powder (0.182 g, 62% yield): ¹H NMR (400 MHz, DMSO) δ 7.812 (s, 1H), 7.459 (d, 1H), 7.147 (s, 1H), 7.128 (d, 1H), 6.915 (d, 1H), 6.620 (m, 3H); m/z 294 (M+H).

### EXAMPLES 60-75

This illustrates the production of aminocyanopyridine compounds of the present invention.

The compounds listed in the table below were prepared by the methods described in Kambe, S. et al., “A simple method for the preparation of 2-amino-4-aryl-3-cyanopyridines by the condensation of malononitrile with aromatic aldehydes and alkyl ketones in the presence of ammonium acetate”, Synthesis 5:366-368 (1980). NMR analysis was carried out for each compound and selected data is presented for each compound as shown in the table.

| Ex. No. | Compound name | m/z (M + H) |
|---|---|---|
| 60 | 4,6-diamino-2-(trifluoromethyl)-2,3- | 245 |
| | dihydrofuro[2,3-b]pyridine-5-carbonitrile or | |
| | 6N009 | |
| 61 | 4,6-diamino-2-(chloromethyl)-2,3- | 225 |
| | dihydrofuro[2,3-b]pyridine-5-carbonitrile | |
| 62 | 4-[2-amino-3-cyano-6-(2-furyl)pyridin-4-yl]- | 295 |
| | 1H-pyrrole-2-carboxylate | |
| 63 | 4,6-diamino-2-[(4- | 313 |
| | methoxyphenoxy)methyl]-2,3- | |
| | dihydrofuro[2,3-b]pyridine-5-carbonitrile | |
| 64 | 4,6-diamino-2-(hydroxymethyl)-2,3- | 207 |
| | dihydrofuro[2,3-b]pyridine-5-carbonitrile | |
| 65 | 2,4-diamino-6-[(4- | 273 |
| | methoxyphenyl)thio]nicotinonitrile | |
| 66 | 4,6-diamino-2-(phenoxymethyl)-2,3- | 283 |
| | dihydrofuro[2,3-b]pyridine-5-carbonitrile | |
| 67 | 4,6-diamino-2-[(2-methylphenoxy)methyl]- | 297 |
| | 2,3-dihydrofuro[2,3-b]pyridine-5-carbonitrile | |
| 68 | 2-amino-7,9-dimethyl-5-oxo-5H- | 266 |
| | chromeno[2,3-b]pyridine-3-carbonitrile | |
| 69 | 2-amino-7-isopropyl-5-oxo-5H- | 280 |
| | chromeno[2,3-b]pyridine-3-carbonitrile | |
| 70 | 2-amino-7-ethyl-5-oxo-5H-chromeno[2,3- | 266 |
| | b]pyridine-3-carbonitrile | |
| 71 | 2-amino-7-methyl-5-oxo-5H-chromeno[2,3- | 252 |
| | b]pyridine-3-carbonitrile | |
| 72 | 2-amino-7-chloro-5-oxo-5H-chromeno[2,3- | 272 |
| | b]pyridine-3-carbonitrile | |
| 73 | 2-amino-7-bromo-5-oxo-5H-chromeno[2,3- | 316, 318 |
| | b]pyridine-3-carbonitrile | |
| 74 | 2-amino-5-oxo-5H-chromeno[2,3- | 238 |
| | b]pyridine-3-carbonitrile | |
| 75 | ethyl 4-[2-amino-3-cyano-6-(2-furyl)pyridin- | 323 |
| | 4-yl]-1H-pyrrole-2-carboxylate | |

### EXAMPLE 76

This illustrates the production of 2-amino-6-(2-furyl)-4-(1H-imidazol-5-yl)nicotinonitrile trifluoroacetate.

Step 1: Production of 2-amino-6-(2-furyl)-4-(1-{[2-(trimethylsilyl)ethoxy]methyl}-1H-imidazol-4-yl)nicotinonitrile.

To a solution of 2-Acetylfuran (0.96 g, 8.71 mmol) and 2-[(1-{[2-(trimethylsilyl)ethoxy]methyl}-1H-imidazol-5-yl)methylene]malononitrile (2.0 g, 7.3 mmol) in benzene (15 mL) at room temperature was added ammonium acetate (1.08 g, 14.1 mmol). After heating to reflux for 10 hrs the reaction was cooled to room temperature and diluted with ethyl acetate and water. The layers were separated and the organic layer washed with brine and dried (Na₂SO₄). The solvent was removed to give a solid, which after chromatography (silica, 30% ethyl acetate/hexane) gave the desired product (0.78 g, 38%). ¹H NMR (300 MHz, d⁶-DMSO) δ 8.14 (s, 1H), 8.02 (s, 1H), 7.88 (s, 1H), 7.57 (s, 1H), 7.10 (d, J =3.3Hz, 1H), 6.81 (bm, 2H), 6.67 (m, 1H), 5.44 (s, 2H), 3.53 (t, J=7.5Hz, 2H), 0.86 (t, J=7.5Hz, 2H), 0.05 (s, 9H): m/z 382 (M+H).

Step 2: Production of 2-amino-6-(2-furyl)-4-(1H-imidazol-5-yl)nicotinonitrile trifluoroacetate.

To a round bottom flask containing 2-amino-6-(2-furyl)-4-(1-{[2-(trimethylsilyl)ethoxy]methyl}-1H-imidazol-4yl)nicotinonitrile (0.42 g, 1.10 mmol), prepared as described in Step 1, above, was added 0.5 M HCl/ethyl alcohol (EtOH) (15 mL) at room temperature. The reaction was heated to reflux for 5 hrs and then allowed to cool. A precipitate formed upon cooling and was filtered. The solid was collected and purified by reverse phase high pressure liquid chromatography (RP-HPLC) (H₂O:CH₃CN+j0.05% TFA) to give the desired product after lypholization (0.22 g, 61% yield). ¹H NMR (300 MHz, d⁶-DMSO) δ 8.46 (bs, 1H), 8.11 (s, 1H), 7.91 (d, J=1.2Hz, 1H), 7.48 (s, 1H), 7.13 (d, J=3.6Hz, 1H), 6.69 (dd, J=1.8, 3.3Hz, 1H), 3.7 (bm, 3H): m/z 252 (M+H).

### EXAMPLE 77

This illustrates the production of ethyl 4-[6-amino-5-cyano-4-(2-fluorophenyl)pyridin-2-yl]benzoate.

To a solution of ethyl 4-acetylbenzoate (1.12 g, 5.83 mmol) and 2-(2-fluorobenzylidene)malononitrile (1.0 g, 5.81 mmol) in benzene at room temperature was added ammonium acetate (0.67 g, 8.69 mmol). The reaction mixture was heated to reflux for 4 hrs and then allowed to cool to room temperature. The reaction mixture was poured into ethanol and the precipitate filtered to give a light yellow solid (0.30 g, 14% yield). ¹H NMR (300 MHz, d⁶-DMSO) δ 8.24 (d, J=8.1Hz, 2H), 8.04 (d, J=8.1 Hz, 2H), 7.60-7.58 (bm, 2H), 7.40-7.34 (bm, 4H), 7.17 (bs, 1H), 4.34 (q, 2H), 1.32 (t, 3H): m/z 362 (M+H).

### EXAMPLE 78

This illustrates the production of 4-[6-amino-5-cyano-4-(2-fluorophenyl)pyridin-2-yl]benzoic acid trifluoroacetate.

To a solution of ethyl-4-[6-amino-5-cyano-4-(2-fluorophenyl)pyridin-2-yl]benzoate (0.20 g, 0.55 mmol) in THF/H₂O (9:1) was added aqueous lithium hydroxide (LiOH.H₂O) at room temperature. The reaction was heated to reflux for 4 hrs and the solvent removed in vacuo to give a solid, which was purified by RP-HPLC to give the desired product (0.091 g, 50% yield). ¹H NMR (300 MHz, d⁶-DMSO) δ 8.27(d, J=8.4 Hz, 2H), 8.08 (d, J=8.4 Hz, 2H), 7.66-7.62 (bm, 2H), 7.52-7.40 (bm, 3H), 7.21 (bs, 1H), 4.81 (bs, 2H): m/z 334 (M+H).

### EXAMPLE 79

This illustrates the production of 2-amino-4-(2-furyl)-6-(1H-pyrazol-3-yl)nicotinonitrile trifluoroacetate.

Step 1: Production of 1-(1H-pyrazol-5-yl)-1-ethanone.

To a solution of potassium hydroxide (KOH) (18 g in 50 mL of water) was added diethyl ether. The solution was cooled to 0° C. and MNNG (1-Methyl-3-1-nitrosoguanidine, 4.0 g) was added slowly to generate CH₂N₂. After this addition was complete the diazomethane (CH₂N₂) in diethyl ether was transferred to a solution of 3-Butyn-2-one (4.0 g, 0.058 mol) in ether via pipet. The reaction was stirred at room temperature for 4 hrs and the solvent removed in vacuo to give an oil, which on high vacuum turned to a solid (1.71 g, 26% yield). ¹H NMR (300 MHz, CDCl₃) δ 7.68 (d, J=2.1 Hz, 1H), 6.84 (d, J=2.1 Hz, 1H), 2.60 (s, 3H).

Step 2: Production of 2-amino-4-(2-furyl)-6-(1H-pyrazol-3-yl)nicotinonitrile trifluoroacetate.

To a solution of 1-(1H-pyrazol-5-yl)-1-ethanone (0.64 g, 5.80 mmol), prepared as described above in Step 1, furaldehyde (0.48 mL, 5.80 mmol), and malononitrile (0.38 g, 5.80 mmol) in benzene (15 mL) at room temperature was added ammonium acetate (1.11 g, 14.5 mmol). The reaction was heated to reflux for 10 hrs and then allowed to cool to room temperature. The mixture was diluted with water and ethyl acetate. The layers were separated and the organic layer washed with brine and dried (Na₂SO₄). The solvent was removed to give a brown solid, which after RP-HPLC (H₂O:CH₃CN+0.05% TFA) gave the desired product (185 mg, 12% yield). ¹H NMR (300 MHz, CD₃OD) δ 8.0 (d, J=1.2 Hz, 1H), 7.81 (d, J=2.1 Hz, 1H), 7.61 (s, 1H), 7.46 (d, J=3.6 Hz, 1H), 6.84 (d, J=2.1 Hz, 1H), 6.78-6.76 (m, 1H); m/z 252 (M+H).

### EXAMPLES 80-91

This illustrates the production of aminocyanopyridine compounds of the present invention.

The compounds listed in the table below were prepared by the methods described in Kambe, S. et al., “A simple method for the preparation of 2-amino-4-aryl-3-cyanopyridines by the condensation of malononitrile with aromatic aldehydes and alkyl ketones in the presence of ammonium acetate”, Synthesis 5:366-368 (1980). NMR analysis was carried out for each compound and selected data is presented for each compound as shown in the table.

| | | m/z |
|---|---|---|
| Ex. No. | Compound name | (M + H) |
| 80 | 2-amino-4-(1H-imidazol-4-yl)-6- | 262 |
| | phenylnicotinonitrile trifluoroacetate hydrate | |
| 81 | 2-amino-4-(2-fluorophenyl)-6-(1H-pyrrol-2- | 279 |
| | yl)nicotinonitrile trifluoroacetate hydrate | |
| 82 | 2-amino-6-(3-chlorophenyl)-4-(1H-imidazol-4- | 296 |
| | yl)nicotinonitrile trifluoroacetate hydrate | |
| 83 | 2-amino-4-(2-fluorophenyl)-6-phenylnicotinonitrile | 290 |
| 84 | ethyl 4-[6-amino-5-cyano-4-(2- | 334 |
| | fluorophenyl)pyridin-2-yl]benzoate | |
| 85 | 2-amino-6-(2-fluorophenyl)-4-(3- | 280 |
| | furyl)nicotinonitrile trifluoroacetate | |
| 86 | 2-amino-4-(2-fluorophenyl)-6-thien-2- | 296 |
| | ylnicotinonitrile hydrate | |
| 87 | 6-amino-4-(2-fluorophenyl)-2,2′-bipyridine-5- | 291 |
| | carbonitrile trifluoroacetate | |
| 88 | 2-amino-4-(2-furyl)-6-(1H-pyrazol-4- | 252 |
| | yl)nicotinonitrile bis(trifluoroacetate) | |
| 89 | 2-amino-4-(2-furyl)-6-(1-trityl-1H-pyrazol-4- | 494 |
| | yl)nicotinonitrile | |
| 90 | 2-amino-4-(2-fluorophenyl)-6-tetrahydrofuran-2- | 284 |
| | ylnicotinonitrile | |
| 91 | ethyl 6-amino-5-cyano-4-(2-fluorophenyl)pyridine- | 286 |
| | 2-carboxylate | |

### EXAMPLE 92

This illustrates the production of 2-amino-4-(2-furyl)-8-hydroxy-5,6-dihydrobenzo[h]quinoline-3-carbonitrile trifluoroacetate.

A glass vial was charged with 6-hydroxy-2-tetralone (0.49 g, 3 mmol), malononitrile, (0. g, 3 mmol), ammonium acetate (0. g, 6 mmol), furaldehyde (0. g, 3 mmol) and a magnetic stirring bar. Benzene (6 mL) was added to the vial, which was capped and heated to 80 degrees Celsius for 18 hours. The vial was then cooled to room temperature, and a 1:2 mixture of methanol and dichloromethane (15 mL) was added followed by 8 g of Amberlyst resin. The mixture was agitated for 24 h, then the resin was filtered and washed with dichloromethane (3×15 mL). A 2 M solution of ammonia in methanol (15 mL) was added to the resin, and the mixture was agitated overnight at room temperature. The resin was filtered and the filtrate collected in a tared flask. The resin was washed sequentially with a 1:1 mixture of methanol and dichloromethane (2×15 mL), 2 M ammonia in methanol (2×15 mL), and a 1:1 mixture of methanol and dichloromethane (2×15 mL). The combined filtrates were concentrated in vacuo, and the residue was purified by reverse phase chromatography. The product was isolated as a tan solid (10.4 mg,1% yield). ¹H NMR (400 MHz, DMSO) δ 2.70 (m, 4H), 6.63 (d, 1H), 6.70 (dd, 1H), 6.73 (d, 1H), 6.87 (d, 1H), 7.91 (d, 1H), 7.96 (d, 1H); m/z 304 (M+H); HRMS (M+H) calculated for C₁₈H₁₄N₃O₂: 304.1086, found 304.1086.

### EXAMPLE 93

This illustrates the production of 2-amino-4-(2-furyl)-6,8-dihydro-5H-pyrrolo[3,4-h]quinoline-3-carbonitrile trifluoroacetate.

This material was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a tan solid (171.9 mg, 17% yield). ¹H NMR (400 MHz, DMSO) δ 2.60 (m, 2H), 2.74 (m, 2H), 6.65 (s, 1H), 6.73 (dd, 1H), 6.90 (d, 1H), 7.30 (s, 1H), 7.95 (s, 1H), 11.9 (br s, 1H); m/z 277 (M+H); HRMS (M+H) calculated for C₁₆H₁₃N₄O: 277.1089, found 277.1078.

### EXAMPLE 94

This illustrates the production of 2-amino-4-(2-furyl)-6,7-dihydro-5H-pyrazolo[3,4-h]quinoline-3-carbonitrile bis(trifluoroacetate).

This material was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a tan solid (248 mg, 17% yield). ¹H NMR (400 MHz, DMSO) δ 2.75-2.90 (m, 4H), 6.73 (dd, 1H), 6.88 (d, 1H), 7.92 (s, 1H), 7.95 (d, 1H); m/z 278 (M+H); HRMS (M+H) calculated for C₁₅H₁₂N₅O: 278.1042, found 278.1058.

### EXAMPLE 95

This illustrates the production of 2-amino-4-(2-fluorophenyl)-5,6-dihydrobenzo[h]quinoline-3-carbonitrile trifluoroacetate.

This material was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a tan solid (49.1 mg, 4% yield). ¹H NMR (400 MHz, DMSO) δ 2.38-2.48 (m, 2H), 2.75-2.82 (m, 2H), 7.25-7.30 (m, 2H), 7.35-7.47 (m, 5H), 7.55-7.64 (m, 1H), 8.16-8.22 (m, 1H); m/z 316 (M+H); ); HRMS (M+H) calculated for C₂₀H₁₅FN₃: 316.1250, found 316.1248.

### EXAMPLE 96

This illustrates the production of 2-amino-3-cyano-4-(2-furyl)-5,6-dihydrobenzo[h]quinoline-8-carboxylic acid trifluoroacetate.

This material was prepared in a manner similar to that used to produce 2-amino-4-(2-furyl)-8-hydroxy-5 ,6-dihydrobenzo[h]quinoline-3-carbonitrile trifluoroacetate, as described in Example 92. The product was isolated as a tan solid (30.1 mg, 5% yield). ¹H NMR (400 MHz, DMSO) δ 2.80-2.93 (m, 4H), 6.77 (dd, 1H), 6.98 (dd, 7.87 (dd, 1H), 7.92 (d, 1H), 7.95 (d, 1H), 7.99 (dd, 1H), 8.23 (d, 1H) ); m/z 332 (M+H); HRMS (M+H) calculated for C₁₉H₁₄N₃O₃: 332.1035, found 332.1032.

### EXAMPLE 97

This illustrates the production of 2-amino-3-cyano-4-(4H-1,2,4-triazol-3-yl)-5,6-dihydrobenzo[h]quinoline-8-carboxylic acid bis(trifluoroacetate).

This material was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a tan solid (29.4 mg, 4% yield). ¹H NMR (400 MHz, DMSO) δ 2.72-2.92 (m, 4H), 7.86 (s, 1H), 7.94 (d, 1H), 8.27 (d, 1H), 8.78 (br s, 1H); m/z 333 (M+H); HRMS (M+H) calculated for C₁₇H₁₃N₆O₂: 333.1100, found 333.1083.

### EXAMPLE 98

This illustrates the production of 2-amino-4-(2-furyl)-5,6-dihydro-1,8-phenanthroline-3-carbonitrile bis(trifluoroacetate).

2-amino-4-(2-furyl)-5,6-dihydro-1,8-phenanth roline-3-carbonitrile bis(trifluoroacetate) was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a tan solid (205 mg, 12% yield). ¹H NMR (400 MHz, DMSO) δ 2.85-2.98 (m, 4H), 6.79 (dd, 1H), 7.04 (dd, 1H), 8.02 (dd, 1H), 8.19 (1H), 8.76 (d, 1H), 8.77 (s, 1H); m/z 289 (M+H); HRMS (M+H) calculated for C₁₇H₁₃N₄O: 289.1089, found 289.1069.

### EXAMPLE 99

This illustrates the production of 2-amino-4-(2-fluorophenyl)-6,8-dihydro-5H-pyrazolo[3,4-h]quinoline-3-carbonitrile bis(trifluoroacetate).

2-amino-4-(2-fluorophenyl)-6,8-dihydro-5H-pyrazolo[3,4-h]quinoline-3-carbonitrile bis(trifluoroacetate) was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a yellow solid (173.7 mg, 17% yield). ¹H NMR (400 MHz, DMSO) δ 2.50-2.60 (m, 2H), 2.72-2.78 (m, 2H), 7.36-7.48 (m, 3H), 7.55-7.63 (m, 1H), 7.97 (s, 1H); m/z 306 (M+H); HRMS (M+H) calculated for C₁₇H₁₃FN₅: 306.1150, found 306.1178.

### EXAMPLE 100

This illustrates the production of 2-amino-4-phenyl-6,8-dihydro-5H-pyrazolo[3,4-h]quinoline-3-carbonitrile bis(trifluoroacetate).

This material was prepared in a manner similar to that used to produce 2-amino-4-(2-furyl) -8-hydroxy-5,6-dihydrobenzo[h]quinoline-3-carbonitrile trifluoroacetate, as described in Example 92. The product was isolated as a yellow solid (242 mg, 24% yield). ¹H NMR (400 MHz, DMSO) δ 2.50-2.62 (m, 2H), 2.69-2.76 (m, 2H), 7.36-7.46 (m, 2H), 7.50-7.59 m, 3H), 7.96 (s, 1H); m/z 288 (M+H); HRMS (M+H) calculated for C₁₇H₁₄N₅: 288.1244, found 288.1253. TNFα release assay IC₅₀=17.7 μM.

### EXAMPLE 101

This illustrates the production of 2-amino-3-cyano-4-(2-furyl)-5,6-dihydrobenzo[h]quinoline-8-carboxylic acid trifluoroacetate.

Step 1: (Preparation of 5-oxo-5,6,7,8-tetrahydronaphthalene-2-yl-trifluoromethanesulfonate)—A round bottomed flask was charged with 6-hydroxy-1-tetralone (7.87 g, 48.5 mmol), pyridine (97 mL) and a magnetic stirring bar. The flask was sealed under nitrogen, and triflic anhydride (8.24 mL, 49 mmol) was added dropwise over 30 minutes. The mixture was stirred at room temperature for 7 days, then the mixture was diluted with diethyl ether. The organic layer was washed with water (1×100 ml), 5% aqueous hydrogen chloride (2×100 mL), and brine (1×100 mL). The organic layer was then dried over magnesium sulfate and concentrated in vacuo. The product was purified via flash column chromatography (0-20% ethyl acetate/hexane) to give 11.72 g of product as a white solid (81% yield). ¹H NMR (400 MHz, DMSO) δ 2.22 (quintet, 2H), 2.72 (t, 2H), 3.06 (t, 2H), 7.22 (s, 1H), 7.24 (d, 1H), 8.17 (d, 1H); HRMS (M+H) calculated for C₁₇H₁₀F₃O₅S: 295.0246, found 295.0285.

Step 2: (Preparation of methyl 5-oxo-5,6,7,8-tetrahydronaphthalene-2-carboxylate)—A three-necked round bottomed flask was charged with5-oxo-5,6,7,8-tetrahydronaphthalene-2-yl-trifluoromethanesulfonate, prepared as described in Step 1, (9.98 g, 33.9 mmol), bis(diphenylphosphonyl)propane (0.42 4,1 mmol), palladium acetate (0.23 g, 1 mmol), methanol (34 mL), dimethylformamide (68 mL), triethylamine (9.5 mL, 68.3 mmol) and a magnetic stirring bar. The flask was fitted with a condenser and septa, then carbon monoxide was bubbled through the solution for 15 minutes. The flask was placed under a nitrogen atmosphere and heated to 70 degrees Celsius for 8 hours. The mixture was diluted with ethyl acetate (200 mL) and washed with water (1×100 mL), 5% aqueous hydrogen chloride (2×200 mL) and brine (1×100 mL). The organic layer was dried over magnesium sulfate and concentrated in vacuo. The residue was purified by flash column chromatography (0-30% ethyl acetate/hexane) to give 4.08 g of product as a yellow solid (59% yield). ¹H NMR (400 MHz, DMSO) δ 2.21 (quintet, 2H), 2.74 (t, 2H), 3.06 (t, 2H), 3.98 (s, 3h), 7.30 (s, 1H), 7.97 (d, 1H), 7.99 (s, 1H), 8.12 (d, 1H); m/z 205 (M+H); HRMS (M+H) calculated for C₁₂H₁₃O₃: 205.0859, found 205.0882.

Step 3: (Preparation of 2-amino-3-cyano-4-(2-furyl)-5,6-dihydrobenzo[h]quinoline-8-carboxylic acid trifluoroacetate)—A glass vial was charged with methyl 5-oxo-5,6,7,8-tetrahydronaphthalene-2-carboxylate, as prepared in Step 2, above, (1.03 g, 5.06 mmol), malononitrile (0.363, 5.5 mmol), 2-furaldehyde (0.42 mL, 5.07 mmol), ammonium acetate (0.794 g, 10.3 mmol), toluene (10 mL) and a magnetic stirring bar. The vial was capped and heated to 80 degrees Celsius for 24 hours. The vial was cooled to room temperature, then the reaction mixture was diluted with a 1:1 mixture of dichloromethane/methanol (20 mL), and amberlyst resin (20 g) was added to the flask. The slurry was agitated for 72 hours at room temperature, then the resin was collected by vacuum filtration and washed with dichloromethane (3×30 mL). The resin was then combined with 2 M ammonia in methanol and agitated for 4 hours at room temperature. The resin was filtered and washed with a 1:1 mixture of dichloromethane/2M ammonia in methanol (6×30 mL). The combined filtrates were concentrated in vacuo. The residue was treated with ethanol (6 mL) and 2 M aqueous lithium hydroxide (6 mL), at 50 degrees Celsius for 1 hour. The mixture was concentrated in vacuo, and the residue purified by preparative reversed-phase HPLC giving 0.3 g of product as a white solid (18% yield). ¹H NMR (300 MHz, DMSO) δ 2.80-2.96 (m, 4H), 6.79 (m, 1H), 7.00 (d, 1H), 7.89 (s, 1H), 7.95 (d, 1H), 8.01 (s, 1H), 8.26 (s, 1H); m/z 332 (M+H); HRMS (M+H) calculated for C₁₉H₁₄N₃O₃: 332.1030, found 332.1039.

### EXAMPLE 102

This illustrates the preparation of 2-amino-4-(2,3-difluorophenyl)-6,7-dihydro-5H-pyrazolo[3,4-h]quinoline-3-carbonitrile bis(trifluoroacetate).

2-amino-4-(2,3-difluorophenyl)-6,7-dihydro-5H-pyrazolo[3,4-h]quinoline-3-carbonitrile bis(trifluoroacetate) was prepared in a manner similar to that used to produce 2-amino-4-(2-furyl)-8-hydroxy-5,6-dihydrobenzo[h]quinoline-3-carbonitrile trifluoroacetate, as described in Example 106. The product was isolated as a yellow solid (205.7 mg, 17% yield). ¹H NMR (400 MHz, DMSO) δ 2.55-2.60 (m, 2H), 2.72-2.80 (m, 2H), 6.81 (br s, 1H), 7.25-7.32 (m, 1H), 7.38-7.46 (m, 1H), 7.58-7.68 (m, 1H), 7.97 (s, 1H); m/z 324 (M+H); HRMS (M+H) calculated for C₁₇H₁₂F₂N₅: 324.1055, found 324.1030. TNFα release assay IC₅₀=4.0 μM; Rat LPS Assay 83% inhibition at 20 mpk (IG).

### EXAMPLE 103

This illustrates the preparation of 2-amino-4-(2,4-difluorophenyl)-6,7-dihydro-5H-pyrazolo[3,4-h]quinoline-3-carbonitrile bis(trifluoroacetate).

2-amino-4-(2,4-difluorophenyl)-6,7-dihydro-5H-pyrazolo[3,4-h]quinoline-3-carbonitrile bis(trifluoroacetate) was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a yellow solid (149.1 mg, 13% yield). ¹H NMR (400 MHz, DMSO) δ 2.55-2.60 (m, 2H), 2.72-2.80 (m, 2H), 6.78 (br s, 1H), 7.31 (td, 1H), 7.47-7.58 (m, 2H), 7.96 (s, 1H); m/z 324 (M+H); HRMS (M+H) calculated for C₁₇H₁₂F₂N₅: 324.1055, found 324.1074.

### EXAMPLE 104

This illustrates the preparation of 2-amino-4-(2,6-difluorophenyl)-6,7-dihydro-5H-pyrazolo[3,4-h]quinoline-3-carbonitrile bis(trifluoroacetate).

2-amino-4-(2,6-difluorophenyl)-6,7-dihydro-5H-pyrazolo[3,4-h]quinoline-3-carbonitrile bis(trifluoroacetate) was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a white solid (137.7 mg, 12% yield). ¹H NMR (400 MHz, DMSO) δ 2.55-2.60 (m, 2H), 2.72-2.80 (m, 2H), 6.85 (br s, 1H), 7.33-7.40 (m, 2H), 7.62-7.73 (m, 1H), 7.98 (s, 1H); m/z 324 (M+H); HRMS (M+H) calculated for C₁₇H₁₂F₂N₅: 324.1055, found 324.1098.

### EXAMPLE 105

This illustrates the preparation of 8-amino-6-(2-furyl)-4,5-dihydro-1H-pyrazolo[4,3-h]quinoline-7-carbonitrile.

8-amino-6-(2-furyl)-4,5-dihydro-1H-pyrazolo[4,3-h]quinoline-7-carbonitrile was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a yellow solid (51 mg, 8% yield). ¹H NMR (400 MHz, DMSO) δ 2.67 (t, 2H), 2.83 (t, 2H), 6.76 (dd, 1H), 6.93 (d, 1H), 7.57 (s, 1H), 7.98 (d, 1H); m/z 278 (M+H); HRMS (M+H) calculated for C₁₅₇H₁₂N₅O: 278.101036, found 278:1051. TNFα release assay IC₅₀=0.9 μM.

### EXAMPLE 106

This illustrates the preparation of 2-amino-4-(2-furyl)-6-(1H-pyrazol-3-yl)nicotinonitrile trifluoroacetate.

2-amino-4-(2-furyl)-6-(1H-pyrazol-3-yl)nicotinonitrile trifluoroacetate was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a brown solid (110 mg, 6% yield). ¹H NMR (300 MHz, DMSO) δ 6.76 (dd, 1H), 6.84 (br s, 1H), 6.95 (s, 1H), 7.46 (d, 1H), 7.64 (s, 1H), 7.86 (s, 1H), 8.03 (s, 1H); m/z 253 (M+H); HRMS (M+H) calculated for C₁₃H₁₀ N₅O: 252.0880, found 252.0855. TNFα release assay IC₅₀=4.0 μM.

### EXAMPLE 107

This illustrates the preparation of 8-amino-6-(2-furyl)-4,5-dihydro-1H-pyrazolo[4,3-h]quinoline-7-carbonitrile trifluoroacetate.

8-amino-6-(2-furyl)-4,5-dihydro-1H-pyrazolo[4,3-h]quinoline-7-carbonitrile trifluoroacetate was prepared in a manner similar to that used to produce 2-amino-4-(2-furyl)-8-hydroxy-5,6-dihydrobenzo[h]quinoline-3-carbonitrile trifluoroacetate, as described in Example 92. The product was isolated as a tan solid (379 mg, 38% yield). ¹H NMR (300 MHz, DMSO) δ 2.69 (t, 2H), 2.84 (t, 2H), 6.76 (dd, 1H), 6.94 dd, 1H), 7.58 (s, 1H), 7.99 (dd, 1H); m/z 278 (M+H); HRMS (M+H) calculated for C₁₅H₁₂N₅O: 278.1036, found 278.1054.

### EXAMPLES 108-174

This illustrates the production of aminocyanopyridine compounds of the present invention.

The compounds listed in the table below were prepared by the methods described in Kambe, S. et al., “A simple method for the preparation of 2-amino-4-aryl-3-cyanopyridines by the condensation of malononitrile with aromatic aldehydes and alkyl ketones in the presence of ammonium acetate”, Synthesis 5:366-368 (1980). NMR analysis was carried out for each compound and selected data is presented for each compound as shown in the table.

| Ex. | | m/z | HRMS | HRMS | Formula |
|---|---|---|---|---|---|
| No. | Compound Name | (M + H) | Theor. | Found | Calcd for |
| 108 | 2-amino-4-(3- | 306 | 306.115 | 306.1168 | C₁₇H₁₃FN₅ |
| | fluorophenyl)-6,8- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 109 | N-{4-[6-amino-5- | 355 | 355.0859 | 355.0853 | C₁₇H₁₅N₄O₃S |
| | cyano-4-(2- | | | | |
| | furyl)pyridin-2- | | | | |
| | yl]phenyl}methanesulfonamide | | | | |
| | trifluoroacetate | | | | |
| 110 | 2-amino-4-(2-furyl)- | 377 | 277.1089 | 277.1063 | C₁₆H₁₃N₄O |
| | 6,7-dihydro-5H- | | | | |
| | pyrrolo[2,3- | | | | |
| | h]quinoline-3- | | | | |
| | carbonitrile | | | | |
| | trifluoroacetate | | | | |
| 111 | 2-amino-4-(4- | 318 | 318.1349 | 318.1349 | C₁₈H₁₆N₅O |
| | methoxyphenyl)- | | | | |
| | 6,7-dihydro-5H- | | | | |
| | pyrazolo[3,4- | | | | |
| | h]quinoline-3- | | | | |
| | carbonitrile | | | | |
| | bis(trifluoroacetate) | | | | |
| 112 | 2-amino-4-(2,5- | 324 | 324.1055 | 324.1098 | C₁₇H₁₂F₂N₅ |
| | difluorophenyl)-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 113 | 2-amino-4-(4- | 306 | 306.115 | 306.1155 | C₁₇H₁₃FN₅ |
| | fluorophenyl)-6,8- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 114 | 2-amino-4-(4H- | 289 | 289.1202 | 289.1173 | C₁₆H₁₃N₆ |
| | 1,2,4-triazol-3-yl)- | | | | |
| | 5,6- | | | | |
| | dihydrobenzo[h]quinoline- | | | | |
| | 3-carbonitrile | | | | |
| | bis(trifluoroacetate) | | | | |
| 115 | 2-amino-6-(4- | 293 | 293.1151 | 293.1137 | C₁₅H₁₃N₆O |
| | methoxyphenyl)-4- | | | | |
| | (4H-1,2,4-triazol-3- | | | | |
| | yl)nicotinonitrile | | | | |
| | bis(trifluoroacetate) | | | | |
| 116 | 2-amino-4-(2- | 280 | 280.0881 | 280.0916 | C₁₆H₁₁FN₃O |
| | fluorophenyl)-6-(3- | | | | |
| | furyl)nicotinonitrile | | | | |
| | trifluoroacetate | | | | |
| 117 | 8-amino-6-(2-furyl)- | 278 | 278.1036 | 278.1018 | C₁₅H₁₂N₅O |
| | 4,5-dihydro-2H- | | | | |
| | pyrazolo[4,3- | | | | |
| | h]quinoline-7- | | | | |
| | carbonitrile | | | | |
| 118 | 2-amino-4-(3- | 318 | 318.1349 | 318.1361 | C₁₈H₁₆N₅O |
| | methoxyphenyl) | | | | |
| | 6,7-dihydro-5H- | | | | |
| | pyrazolo[3,4- | | | | |
| | h]quinoline-3- | | | | |
| | carbonitrile | | | | |
| | bis(trifluoroacetate) | | | | |
| 119 | 2-amino-4-(2-furyl)- | 292 | 292.1198 | 292.1201 | C₁₆H₁₄N₅O |
| | 7-methyl-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 120 | N-[4-(2-amino-3- | 303 | 303.1353 | 303.1399 | C₁₉H₁₇N₆O |
| | cyano-6,7-dihydro- | | | | |
| | 5H-pyrazolo[3,4- | | | | |
| | h]quinolin-4- | | | | |
| | yl)phenyl]acetamide | | | | |
| | bis(trifluoroacetate) | | | | |
| 121 | 6-amino-4-[(4- | 351 | 351.1063 | 351.1078 | C₁₆H₁₄F₃N₄O₂ |
| | methoxyphenyl)amino]- | | | | |
| | 2- | | | | |
| | (trifluoromethyl)- | | | | |
| | 2,3-dihydrofuro[2,3- | | | | |
| | b]pyridine-5- | | | | |
| | carbonitrile | | | | |
| 122 | 4,6-diamino-2- | 205 | 205.1089 | 205.1056 | C₁₀H₁₃N₄O |
| | ethyl-2,3- | | | | |
| | dihydrofuro[2,3- | | | | |
| | b]pyridine-5- | | | | |
| | carbonitrile | | | | |
| | trifluoroacetate | | | | |
| 123 | 3-(2-amino-3- | 332 | 332.1142 | 332.1148 | C₁₈H₁₄N₅O₂ |
| | cyano-6,7-dihydro- | | | | |
| | 5H-pyrazolo[3,4- | | | | |
| | h]quinolin-4- | | | | |
| | yl)benzoic acid | | | | |
| | bis(trifluoroacetate) | | | | |
| 124 | 2-amino-4-(1,3- | 332 | 332.1142 | 332.1124 | C₁₈H₁₄N₅O₂ |
| | benzodioxol-4-yl)- | | | | |
| | 6,7-dihydro-5H- | | | | |
| | pyrazolo[3,4- | | | | |
| | h]quinoline-3- | | | | |
| | carbonitrile | | | | |
| | bis(trifluoroacetate) | | | | |
| 125 | 4,6-diamino-2- | 191 | 191.0933 | 191.0896 | C₉H₁₁N₄O |
| | methyl-2,3- | | | | |
| | dihydrofuro[2,3- | | | | |
| | b]pyridine-5- | | | | |
| | carbonitrile | | | | |
| | trifluoroacetate | | | | |
| 126 | 2,8-diamino-4-(2- | 303 | 303.1246 | 303.1237 | C₁₈H₁₅N₄O |
| | furyl)-5,6- | | | | |
| | dihydrobenzo[h]quinoline- | | | | |
| | 3-carbonitrile | | | | |
| | trifluoroacetate | | | | |
| 127 | 4,6-diamino-2- | 233 | 233.1402 | 233.1378 | C₁₂H₁₇N₄O |
| | butyl-2,3- | | | | |
| | dihydrofuro[2,3- | | | | |
| | b]pyridine-5- | | | | |
| | carbonitrile | | | | |
| | trifluoroacetate | | | | |
| 128 | 2-amino-4-(4- | 313 | 313.1196 | 313.1244 | C₁₈H₁₃N₆ |
| | cyanophenyl)-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 129 | 2-amino-4-(2- | 322 | 322.0854 | 322.089 | C₁₇H₁₃CIN₅ |
| | chlorophenyl)-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 130 | 2-amino-4-pyridin- | 289 | 289.1196 | 289.1209 | C₁₆H₁₃N₆ |
| | 3-yl-6,8-dihydro- | | | | |
| | 5H-pyrazolo[3,4- | | | | |
| | h]quinoline-3- | | | | |
| | carbonitrile | | | | |
| | tris(trifluoroacetate) | | | | |
| 131 | 2-amino-4-(2-furyl)- | 304 | 304.1086 | 304.1076 | C₁₈H₁₄N₃O₂ |
| | 7-hydroxy-5,6- | | | | |
| | dihydrobenzo[h]quinoline- | | | | |
| | 3-carbonitrile | | | | |
| | trifluoroacetate | | | | |
| 132 | 2-amino-4-(2-furyl)- | 301 | 301.1084 | 301.1078 | C₁₈H₁₃N₄O |
| | 6-(1H-indol-3- | | | | |
| | yl)nicotinonitrile | | | | |
| | trifluoroacetate | | | | |
| 133 | 2-amino-4-pyridin- | 289 | 289.1196 | 289.1218 | C₁₆H₁₃N₆ |
| | 4-yl-6,8-dihydro- | | | | |
| | 5H-pyrazolo[3,4- | | | | |
| | h]quinoline-3- | | | | |
| | carbonitrile | | | | |
| | tris(trifluoroacetate) | | | | |
| 134 | 2-amino-4-[2- | 354 | 354.1161 | 354.1162 | C₁₈H₁₄F₂N₅O |
| | (difluoromethoxy)phenyl]- | | | | |
| | 6,7-dihydro- | | | | |
| | 5H-pyrazolo[3,4- | | | | |
| | h]quinoline-3- | | | | |
| | carbonitrile | | | | |
| | bis(trifluoroacetate) | | | | |
| 135 | 4,6-diamino-2- | 245 | 245.1039 | 245.1019 | C₁₂H₁₃N₄O₂ |
| | [(prop-2- | | | | |
| | ynyloxy)methyl]- | | | | |
| | 2,3-dihydrofuro[2,3- | | | | |
| | b]pyridine-5- | | | | |
| | carbonitrile | | | | |
| | trifluoroacetate | | | | |
| 136 | 2-[(allyloxy)methyl]- | 247 | 247.1195 | 247.1179 | C₁₂H₁₅N₄O₂ |
| | 4,6-diamino-2,3- | | | | |
| | dihydrofuro[2,3- | | | | |
| | b]pyridine-5- | | | | |
| | carbonitrile | | | | |
| | trifluoroacetate | | | | |
| 137 | 4,6-diamino-2- | 221 | 221.1039 | 221.1015 | C₁₀H₁₃N₄O₂ |
| | (methoxymethyl)- | | | | |
| | 2,3-dihydrofuro[2,3- | | | | |
| | b]pyridine-5- | | | | |
| | carbonitrile | | | | |
| | trifluoroacetate | | | | |
| 138 | 2-amino-4-(2-furyl)- | 302 | 302.1293 | 302.1269 | C₁₉H₁₆N₃O |
| | 6-methyl-5,6- | | | | |
| | dihydrobenzo[h]quinoline- | | | | |
| | 3-carbonitrile | | | | |
| | trifluoroacetate | | | | |
| 139 | 4,6-diamino-2- | 249 | 249.1352 | 249.1336 | C₁₂H₁₇N₄O₂ |
| | (isopropoxymethyl)- | | | | |
| | 2,3-dihydrofuro[2,3- | | | | |
| | b]pyridine-5- | | | | |
| | carbonitrile | | | | |
| | trifluoroacetate | | | | |
| 140 | 4,6-diamino-2- | 235 | 235.1195 | 235.118 | C₁₁H₁₅N₄O₂ |
| | (ethoxymethyl)-2,3- | | | | |
| | dihydrofuro[2,3- | | | | |
| | b]pyridine-5- | | | | |
| | carbonitrile | | | | |
| | trifluoroacetate | | | | |
| 141 | 4,6-diamino-2- | 307 | 307.0813 | 307.0819 | C₁₁H₁₁F₄N₄O₂ |
| | [(1,1,2,2- | | | | |
| | tetrafluoroethoxy)methyl]- | | | | |
| | 2,3- | | | | |
| | dihydrofuro[2,3- | | | | |
| | b]pyridine-5- | | | | |
| | carbonitrile | | | | |
| 142 | 2-amino-4-(2- | 318 | 318.1349 | 318.1357 | C₁₈H₁₆N₅O |
| | methoxyphenyl)- | | | | |
| | 6,8-dihydro-5H- | | | | |
| | pyrazolo[3,4- | | | | |
| | h]quinoline-3- | | | | |
| | carbonitrile | | | | |
| | bis(trifluoroacetate) | | | | |
| 143 | 4-(2-amino-3- | 332 | 332.1142 | 332.1153 | C₁₈H₁₄N₅O₂ |
| | cyano-6,7-dihydro- | | | | |
| | 5H-pyrazolo[3,4- | | | | |
| | h]quinolin-4- | | | | |
| | yl)benzoic acid | | | | |
| | bis(trifluoroacetate) | | | | |
| 144 | 4,6-diamino-2-(tert- | 263 | 263.1503 | 263.1506 | C₁₃H₁₉N₄O₂ |
| | butoxymethyl)-2,3- | | | | |
| | dihydrofuro[2,3- | | | | |
| | b]pyridine-5- | | | | |
| | carbonitrile | | | | |
| 145 | methyl 3-(2-amino- | 346 | 346.1299 | 346.1318 | C₁₉H₁₆N₅O₂ |
| | 3-cyano-6,7- | | | | |
| | dihydro-5H- | | | | |
| | pyrazolo[3,4- | | | | |
| | h]quinolin-4- | | | | |
| | yl)benzoate | | | | |
| | bis(trifluoroacetate) | | | | |
| 146 | 4,6-diamino-3- | 253 | 253.1038 | 253.1082 | C₁₄H₁₃N₄O |
| | phenyl-2,3- | | | | |
| | dihydrofuro[2,3- | | | | |
| | b]pyridine-5- | | | | |
| | carbonitrile | | | | |
| | trifluoroacetate | | | | |
| 147 | 4,6-diamino-3-vinyl- | 203 | 203.0933 | 203.0904 | C₁₀H₁₁N₄O |
| | 2,3-dihydrofuro[2,3- | | | | |
| | b]pyridine-5- | | | | |
| | carbonitrile | | | | |
| | trifluoroacetate | | | | |
| 148 | 4,6-diamino-2- | 283 | 283.1167 | 283.1195 | C₁₅H₁₅N₄O₂ |
| | (phenoxymethyl)- | | | | |
| | 2,3-dihydrofuro[2,3- | | | | |
| | b]pyridine-5- | | | | |
| | carbonitrile | | | | |
| | trifluoroacetate | | | | |
| 149 | 2-amino-4-(2-furyl)- | 316 | 316.145 | 316.1441 | C₂₀H₁₈N₃O |
| | 7,9-dimethyl-5,6- | | | | |
| | dihydrobenzo[h]quinoline- | | | | |
| | 3-carbonitrile | | | | |
| | trifluoroacetate | | | | |
| 150 | 2-amino-4-(2-furyl)- | 318 | 318.1243 | 318.124 | C₁₉H₁₆N₃O₂ |
| | 7-methoxy-5,6- | | | | |
| | dihydrobenzo[h]quinoline- | | | | |
| | 3-carbonitrile | | | | |
| | trifluoroacetate | | | | |
| 151 | 2-amino-4-(2-furyl)- | 348 | 348.1348 | 348.1351 | C₂₀H₁₈N₃O₃ |
| | 8,9-dimethoxy-5,6- | | | | |
| | dihydrobenzo[h]quinoline- | | | | |
| | 3-carbonitrile | | | | |
| | trifluoroacetate | | | | |
| 152 | 2-amino-4-(2-furyl)- | 318 | 318.1243 | 318.1232 | C₁₉H₁₆N₃O₂ |
| | 8-methoxy-5,6- | | | | |
| | dihydrobenzo[h]quinoline- | | | | |
| | 3-carbonitrile | | | | |
| | trifluoroacetate | | | | |
| 153 | 2-amino-4-(2-furyl)- | 318 | 318.1243 | 318.1243 | C₁₉H₁₆N₃O₂ |
| | 9-methoxy-5,6- | | | | |
| | dihydrobenzo[h]quinoline- | | | | |
| | 3-carbonitrile | | | | |
| | trifluoroacetate | | | | |
| 154 | 2-amino-4-(2-furyl)- | 274 | 274.098 | 274.1051 | C₁₇H₁₂N₃O |
| | 5H-indeno[1,2- | | | | |
| | b]pyridine-3- | | | | |
| | carbonitrile | | | | |
| | trifluoroacetate | | | | |
| 155 | 2-amino-4-(2-furyl)- | 302 | 302.1293 | 302.1285 | C₁₉H₁₆N₃O |
| | 6,7-dihydro-5H- | | | | |
| | benzo[6,7]cyclohepta | | | | |
| | [1,2-b]pyridine-3- | | | | |
| | carbonitrile | | | | |
| | trifluoroacetate | | | | |
| 156 | 2-amino-4-(3- | 316 | 316.125 | 316.149 | C₂₀H₁₅FN₃ |
| | fluorophenyl)-5,6- | | | | |
| | dihydrobenzo[h]quinoline- | | | | |
| | 3-carbonitrile | | | | |
| | trifluoroacetate | | | | |
| 157 | 2-amino-4-(2- | 332 | 332.1506 | 332.1507 | C₁₉H₁₈N₅O |
| | ethoxyphenyl)-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 158 | methyl[2-(2-amino- | 376 | 376.1404 | 376.1403 | C₂₀H₁₈N₅O₃ |
| | 3-cyano-6,7- | | | | |
| | dihydro-5H- | | | | |
| | pyrazolo[3,4- | | | | |
| | h]quinolin-4- | | | | |
| | yl)phenoxy]acetate | | | | |
| | bis(trifluoroacetate) | | | | |
| 159 | 4-[2- | 344 | 344.1506 | 344.1507 | C₂₀H₁₈N₅O |
| | (allyloxy)phenyl]-2- | | | | |
| | amino-6,7-dihydro- | | | | |
| | 5H-pyrazolo[3,4- | | | | |
| | h]quinoline-3- | | | | |
| | carbonitrile | | | | |
| | bis(trifluoroacetate) | | | | |
| 160 | 2-amino-4-[2-(beta- | 466 | 466.1721 | 466.1742 | C₂₃H₂₄N₅O₆ |
| | D- | | | | |
| | glucopyranosyloxy) | | | | |
| | phenyl]-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 161 | 2-amino-4-[2- | 388 | 388.2132 | 388.2136 | C₂₃H₂₆N₅O |
| | (hexyloxy)phenyl]- | | | | |
| | 6,7-dihydro-5H- | | | | |
| | pyrazolo[3,4- | | | | |
| | h]quinoline-3- | | | | |
| | carbonitrile | | | | |
| | bis(trifluoroacetate) | | | | |
| 162 | methyl 2-(2-amino- | 346 | 346.1299 | 346.1345 | C₁₉H₁₆N₅O₂ |
| | 3-cyano-6,7- | | | | |
| | dihydro-5H- | | | | |
| | pyrazolo[3,4- | | | | |
| | h]quinolin-4- | | | | |
| | yl)benzoate | | | | |
| | bis(trifluoroacetate) | | | | |
| 163 | 2-amino-4-(1H- | 327 | 327.1353 | 327.164 | C₁₉H₁₅N₆ |
| | indol-7-yl)-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 164 | methyl 4-(2-amino- | 346 | 346.1299 | 346.1329 | C₁₉H₁₆N₅O₂ |
| | 3-cyano-6,7- | | | | |
| | dihydro-5H- | | | | |
| | pyrazolo[3,4- | | | | |
| | h]quinolin-4- | | | | |
| | yl)benzoate | | | | |
| | bis(trifluoroacetate) | | | | |
| 165 | 2-amino-4-[4- | 331 | 331.1666 | 331.1684 | C₁₉H₁₉N₆ |
| | (dimethylamino)phenyl]- | | | | |
| | 6,7-dihydro-5H- | | | | |
| | pyrazolo[3,4- | | | | |
| | h]quinoline-3- | | | | |
| | carbonitrile | | | | |
| | bis(trifluoroacetate) | | | | |
| 166 | 2-amino-4-(2- | 302 | 302.14 | 302.1408 | C₁₈H₁₆N₅ |
| | methylphenyl)-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 167 | 2-amino-4-[2-(2- | 348 | 348.1455 | 348.149 | C₁₉H₁₈N₅O₂ |
| | hydroxyethoxy)phenyl]- | | | | |
| | 6,7-dihydro-5H- | | | | |
| | pyrazolo[3,4- | | | | |
| | h]quinoline-3- | | | | |
| | carbonitrile | | | | |
| | bis(trifluoroacetate) | | | | |
| 168 | 2-amino-4-{4-[(2- | 370 | 370.1775 | 370.1754 | C₂₁H₂₀N₇ |
| | cyanoethyl)(methyl) | | | | |
| | amino]phenyl}-6,7- | | | | |
| | dihydro-5H- | | | | |
| | pyrazolo[3,4- | | | | |
| | h]quinoline-3- | | | | |
| | carbonitrile | | | | |
| | bis(trifluoroacetate) | | | | |
| 169 | 2-amino-4-(2-furyl)- | 306 | 306.0696 | 306.07 | C₁₇H₁₂N₃OS |
| | 5H- | | | | |
| | thiochromeno[4,3- | | | | |
| | b]pyridine-3- | | | | |
| | carbonitrile | | | | |
| | trifluoroacetate | | | | |
| 170 | 2-amino-4-[2- | 372 | 372.1067 | 372.1095 | C₁₈H₁₃F₃N₅O |
| | (trifluoromethoxy)phenyl]- | | | | |
| | 6,7-dihydro- | | | | |
| | 5H-pyrazolo[3,4- | | | | |
| | h]quinoline-3- | | | | |
| | carbonitrile | | | | |
| | bis(trifluoroacetate) | | | | |
| 171 | [2-(2-amino-3- | 362 | 362.1248 | 362.1233 | C₁₉H₁₆N₅O₃ |
| | cyano-6,7-dihydro- | | | | |
| | 5H-pyrazolo[3,4- | | | | |
| | h]quinolin-4- | | | | |
| | yl)phenoxy]acetic | | | | |
| | acid | | | | |
| | bis(trifluoroacetate) | | | | |
| 172 | 2-(2-amino-3- | 332 | 332.1142 | 332.1131 | C₁₈H₁₄N₅O₂ |
| | cyano-6,7-dihydro- | | | | |
| | 5H-pyrazolo[3,4- | | | | |
| | h]quinolin-4- | | | | |
| | yl)benzoic acid | | | | |
| | bis(trifluoroacetate) | | | | |
| 173 | 2-amino-4-[2- | 354 | 354.1161 | 354.1163 | C₁₈H₁₄F₂N₅O |
| | (difluoromethoxy)phenyl]- | | | | |
| | 6,7-dihydro- | | | | |
| | 5H-pyrazolo[3,4- | | | | |
| | h]quinoline-3- | | | | |
| | carbonitrile | | | | |
| 174 | 4,6-diamino-2- | 276 | 276.1455 | 276.1455 | C₁₃H₁₈N₅O₂ |
| | (morpholin-4- | | | | |
| | ylmethyl)-2,3- | | | | |
| | dihydrofuro[2,3- | | | | |
| | b]pyridine-5- | | | | |
| | carbonitrile | | | | |

### EXAMPLE 175

This illustrates the preparation of 4-[6-amino-5-cyano-4-(2-furyl)pyridin-2-yl]benzoic acid trifluoroacetate.

A glass vial was charged with 4-acetylbenzoic acid (0.33 g, 2 mmol), malononitrile, (0.12 g, 3 mmol), ammonium acetate (0.23 g, 6 mmol), furaldehyde (0.19 g, 3 mmol) and a magnetic stirring bar. Toluene (3 mL) was added to the vial, which was capped and heated to 80 degrees Celsius for 18 hours. The vial was then cooled to room temperature, and a 1:2 mixture of methanol and dichloromethane (15 mL) was added followed by 8 g of Amberlyst resin. The mixture was agitated for 24 h, then the resin was filtered and washed with dichloromethane (3×15 mL). A 2 M solution of ammonia in methanol (15 mL) was added to the resin, and the mixture was agitated overnight at room temperature. The resin was filtered and the filtrate collected in a tared flask. The resin was washed sequentially with a 1:1 mixture of methanol and dichloromethane (2×15 mL), 2 M ammonia in methanol (2×15 mL), and a 1:1 mixture of methanol and dichloromethane (2×15 mL). The combined filtrates were concentrated in vacuo, and the residue was purified by reverse phase chromatography. The product was isolated as a tan solid (9.1 mg, 1% yield). ¹H NMR (300 MHz, CDCl₃—CD₃OD) δ 6.60 (dd, 1H), 7.49 (d, 1H), 7.54 (s, 1H), 7.663 (d, 1H), 8.02 (d, 2H), 8.12 (d, 2H); m/z 306 (M+H); HRMS (M+H) calculated for C₁₇Hl₃N₃O₃: 306.0879, found 306.0874.

### EXAMPLES 176-213

This illustrates the production of aminocyanopyridine compounds of the present invention.

The compounds listed in the table below were prepared by the methods described in Kambe, S. et al., “A simple method for the preparation of 2-amino-4-aryl-3-cyanopyridines by the condensation of malononitrile with aromatic aldehydes and alkyl ketones in the presence of ammonium acetate”, Synthesis 5:366-368 (1980). NMR analysis was carried out for each compound and selected data is presented for each compound as shown in the table.

| Ex. | | m/z | HRMS | HRMS | Formula |
|---|---|---|---|---|---|
| No. | Compound name | (M + H) | Theor. | Found | Calcd for |
| 176 | 2-amino-4-(2-furyl)- | 283 | 283.1559 | 283.1577 | C₁₆H₁₉N₄O |
| | 6-propyl-5,6,7,8- | | | | |
| | tetrahydro-1,6- | | | | |
| | naphthyridine-3- | | | | |
| | carbonitrile | | | | |
| | bis(trifluoroacetate) | | | | |
| 177 | 2-amino-4-(2-furyl)- | 346 | 346.0803 | 346.0831 | C₁₇H₁₁F₃N₃O₂ |
| | 6-[4- | | | | |
| | (trifluoromethoxy)phenyl] | | | | |
| | nicotinonitrile | | | | |
| | trifluoroacetate | | | | |
| 178 | 2-amino-4-(2-furyl)- | 276 | 276.1137 | 276.116 | C₁₇H₁₄N₃O |
| | 6-methyl-5- | | | | |
| | phenylnicotinonitrile | | | | |
| | trifluoroacetate | | | | |
| 179 | 2-amino-6-benzyl-4- | 276 | 276.1137 | 276.117 | C₁₇H₁₄N₃O |
| | (2-furyl)nicotinonitrile | | | | |
| | trifluoroacetate | | | | |
| 180 | 2-amino-4-(2-furyl)- | 242 | 242.1293 | 242.1319 | C₁₄H₁₆N₃O |
| | 6- | | | | |
| | isobutylnicotinonitrile | | | | |
| 181 | 2-amino-4-(2-furyl)- | 240 | 240.1137 | 240.1154 | C₁₄H₁₄N₃O |
| | 5,6,7,8- | | | | |
| | tetrahydroquinoline- | | | | |
| | 3-carbonitrile | | | | |
| 182 | 2-amino-5-(4- | 294 | 294.1043 | 294.1053 | C₁₇H₁₃FN₃O |
| | fluorophenyl)-4-(2- | | | | |
| | furyl)-6- | | | | |
| | methylnicotinonitrile | | | | |
| | trifluoroacetate | | | | |
| 183 | 2-amino-6-(4- | 294 | 294.1043 | 294.1063 | C₁₇H₁₃FN₃O |
| | fluorobenzyl)-4-(2- | | | | |
| | furyl)nicotinonitrile | | | | |
| | trifluoroacetate | | | | |
| 184 | 2-amino-6-(4- | 280 | 280.0886 | 280.0904 | C₁₆H₁₁FN₃O |
| | fluorophenyl)-4-(2- | | | | |
| | furyl)nicotinonitrile | | | | |
| | trifluoroacetate | | | | |
| 185 | 2-amino-4-(2-furyl)- | 252 | 252.1137 | 252.1136 | C₁₅H₁₄N₃O |
| | 5,6,7,8-tetrahydro- | | | | |
| | 5,8- | | | | |
| | methanoquinoline-3- | | | | |
| | carbonitrile | | | | |
| | trifluoroacetate | | | | |
| 186 | 2-amino-6-(3,4- | 290 | 290.1293 | 290.1292 | C₁₈H₁₆N₃O |
| | dimethylphenyl)-4- | | | | |
| | (2-furyl)nicotinonitrile | | | | |
| | trifluoroacetate | | | | |
| 187 | 2-amino-4-(2-furyl)- | 288 | 288.1137 | 288.1139 | C₁₈H₁₄N₃O |
| | 5,6- | | | | |
| | dihydrobenzo[h]quinoline- | | | | |
| | 3-carbonitrile | | | | |
| | trifluoroacetate | | | | |
| 188 | 2-amino-4-(2-furyl)- | 276 | 276.1137 | 276.1143 | C₁₇H₁₄N₃O |
| | 5-methyl-6- | | | | |
| | phenylnicotinonitrile | | | | |
| | trifluoroacetate | | | | |
| 189 | 2-amino-4-(2-furyl)- | 338 | 338.1293 | 338.1294 | C₂₂H₁₆N₃O |
| | 5,6- | | | | |
| | diphenylnicotinonitrile | | | | |
| | trifluoroacetate | | | | |
| 190 | 2-amino-6-(4- | 294 | 294.1043 | 294.1044 | C₁₇H₁₃FN₃O |
| | fluorophenyl)-4-(2- | | | | |
| | furyl)-5- | | | | |
| | methylnicotinonitrile | | | | |
| | trifluoroacetate | | | | |
| 191 | 2-amino-4-(2-furyl)- | 306 | 306.1243 | 306.1235 | C₁₈H₁₆N₃O₂ |
| | 6-(4- | | | | |
| | methoxyphenyl)-5- | | | | |
| | methylnicotinonitrile | | | | |
| | trifluoroacetate | | | | |
| 192 | 2-amino-4-(2-furyl)- | 278 | 278.093 | 278.093 | C₁₆H₁₂N₃O₂ |
| | 6-(3- | | | | |
| | hydroxyphenyl)nicotinonitrile | | | | |
| | trifluoroacetate | | | | |
| 193 | 2-amino-4-(2-furyl)- | 292 | 292.1086 | 292.1086 | C₁₇H₁₄N₃O₂ |
| | 6-(4-hydroxyphenyl)- | | | | |
| | 5- | | | | |
| | methylnicotinonitrile | | | | |
| | trifluoroacetate | | | | |
| 194 | 2-amino-4-(2-furyl)- | 278 | 278.093 | 278.0934 | C₁₆H₁₂N₃O₂ |
| | 6-(4- | | | | |
| | hydroxyphenyl)nicotinonitrile | | | | |
| | trifluoroacetate | | | | |
| 195 | 2-amino-4-(2-furyl)- | 241 | 241.1089 | 241.1076 | C₁₃H₁₃N₄O₂ |
| | 5,6,7,8-tetrahydro- | | | | |
| | 1,6-naphthyridine-3- | | | | |
| | carbonitrile | | | | |
| | bis(trifluoroacetate) | | | | |
| 196 | 2-amino-4-(2-furyl)- | 328 | 328.1086 | 328.1095 | C₂₀H₁₄N₃O₂ |
| | 6-(8-hydroxy-1- | | | | |
| | naphthyl)nicotinonitrile | | | | |
| | trifluoroacetate | | | | |
| 197 | ethyl 2-amino-3- | 312 | 312.1348 | 312.1342 | C₁₇H₁₈N₃O₂ |
| | cyano-4-(2-furyl)- | | | | |
| | 5,6,7,8- | | | | |
| | tetrahydroquinoline- | | | | |
| | 6-carboxylate | | | | |
| | trifluoroacetate | | | | |
| 198 | 2-amino-6-(4- | 287 | 287.0933 | 287.0941 | C₁₇H₁₁N₄O |
| | cyanophenyl)-4-(2- | | | | |
| | furyl)nicotinonitrile | | | | |
| | trifluoroacetate | | | | |
| 199 | 2-amino-4-(2-furyl)- | 265 | 265.1089 | 265.1123 | C₁₅H₁₃N₄O |
| | 6-(1-methyl-1H- | | | | |
| | pyrrol-2- | | | | |
| | yl)nicotinonitrile | | | | |
| 200 | 2-amino-4,6-di(2- | 252 | 252.0773 | 252.0751 | C₁₄H₁₀N₃O₃ |
| | furyl)nicotinonitrile | | | | |
| 201 | 2-amino-4-(2-furyl)- | 251 | 251.0933 | 251.0928 | C₁₄H₁₁N₄O |
| | 6-(1H-pyrrol-2- | | | | |
| | yl)nicotinonitrile | | | | |
| 202 | 2-amino-4-(2-furyl)- | 328 | 328.1198 | 328.1194 | C₁₉H₁₄N₅O |
| | 6-[4-(1H-imidazol-1- | | | | |
| | yl)phenyl]nicotinonitrile | | | | |
| 203 | 2-amino-4-(2-furyl)- | 269 | 269.0497 | 269.0479 | C₁₃H₉N₄O |
| | 6-(1,3-thiazol-2- | | | | |
| | yl)nicotinonitrile | | | | |
| | bis(trifluoroacetate) | | | | |
| 204 | 2-amino-4-(2-furyl) | 268 | 268.0545 | 268.0545 | C₁₄H₁₀N₃O |
| | 6-thien-3- | | | | |
| | ylnicotinonitrile | | | | |
| 205 | 2-amino-6-(1,3- | 306 | 306.0879 | 306.0888 | C₁₇H₁₂N₃O₃ |
| | benzodioxol-5-yl)-4- | | | | |
| | (2-furyl)nicotinonitrile | | | | |
| 206 | 6-amino-4-(2-furyl)- | 326 | 263.0933 | 263.0945 | C₁₅H₁₁N₄O |
| | 2,2′-bipyridine-5- | | | | |
| | carbonitrile | | | | |
| | bis(trifluoroacetate) | | | | |
| 207 | 6-amino-4-(2-furyl)- | 263 | 263.0933 | 263.0935 | C₁₅H₁₁N₄O |
| | 2,3′-bipyridine-5- | | | | |
| | carbonitrile | | | | |
| 208 | 6-amino-4-(2-furyl)- | 263 | 263.0933 | 263.0928 | C₁₅H₁₁N₄O |
| | 2,4′-bipyridine-5- | | | | |
| | carbonitrile | | | | |
| | bis(trifluoroacetate) | | | | |
| 209 | 2-amino-4-(2-furyl)- | 262 | 262.098 | 262.0971 | C₁₆H₁₂N₃O |
| | 6- | | | | |
| | phenylnicotinonitrile | | | | |
| 210 | 2-amino-4-(2-furyl)- | 276 | 276.1137 | 276.1121 | C₁₇H₁₄N₃O |
| | 6-(4- | | | | |
| | methylphenyl)nicotinonitrile | | | | |
| 211 | 2-amino-4-(2-furyl)- | 265 | 265.1089 | 265.1088 | C₁₅H₁₃N₄O |
| | 6-(1-methyl-1H- | | | | |
| | pyrrol-3- | | | | |
| | yl)nicotinonitrile | | | | |
| 212 | 2-amino-4-(2-furyl)- | 301 | 301.1089 | 301.1107 | C₁₅H₁₃N₄O |
| | 6-(1H-indol-3- | | | | |
| | yl)nicotinonitrile | | | | |
| 213 | 2-amino-4-(2- | 286 | — | — | C₁₈H₁₂N₃O |
| | furyl)benzo[h]quinoline- | | | | |
| | 3-carbonitrile | | | | |
| | trifluoroacetate | | | | |

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. An aminocyanopyridine compound, or a pharmaceutically acceptable salt or tautomer or isomer thereof, the compound having the structure: 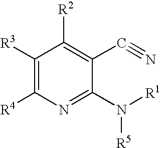
wherein:
R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-( C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;
R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy, 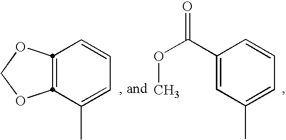
with the proviso that when R² is aryl, it is not substituted with nitro;
R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except that when R² is heteroaryl, R³ is other than cyano, and
where the R² and R³ groups are such that they optionally join to form a ring system selected from: 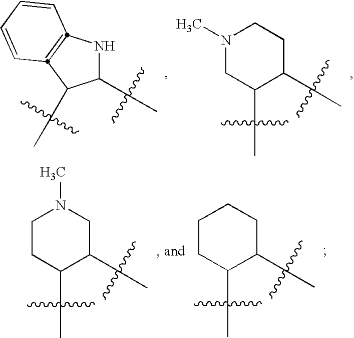
R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups are optionally substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy 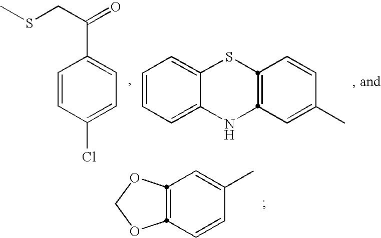
wherein the R³ and R⁴ groups are such that they optionally join to form a ring system selected from: 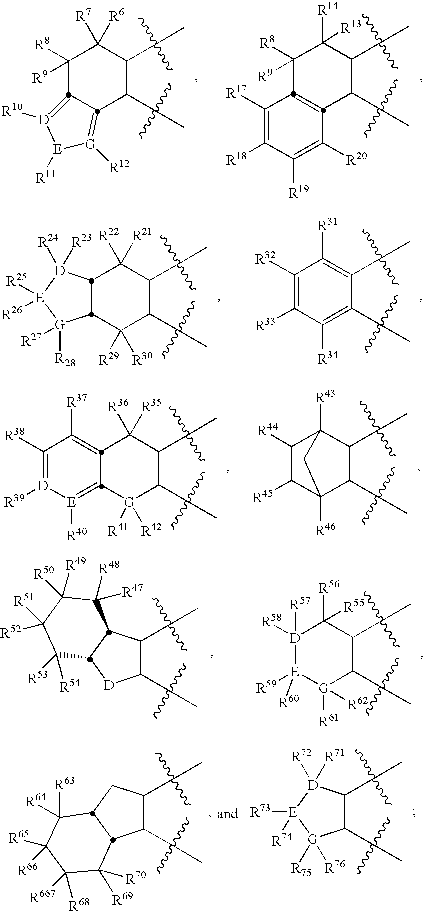
D, E and G are each independently selected from carbon, oxygen, sulfur, and nitrogen;
R⁵ is selected from the group consisting of —H, and C₁-C₅ alkyl, provided that at least one of R¹, R², R³, R⁴ and R⁵ is other than hydrogen; and
wherein the R¹ and R⁵ groups optionally join to form a piperidyl ring or a oxaxinyl ring;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl-ethyl ester, pyridyl C₁-C₄ alkyl, pyridyl C₁-C₄ alkoxy, —COO—CH₂—CH₃, with the proviso that when E is —N—, R³⁸ is other than cyano, and that when G is —N—, R³⁶ is —H; and
wherein R³⁸ and R³⁹ are such that they optionally join to form a ring system of the type selected from: 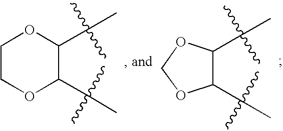
with the proviso that when R¹, R³ and R⁵ are hydrogen:
R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};
where Z₁ and Z₂ are each independently selected from the group consisting of hydrogen, alkoxycarbonyl, alkyl, alkylcarbonyl, benzyl, benzyloxycarbonyl, and formyl;
R^{A} is selected from the group consisting of aryl and arylalkyl;
R^{B} is selected from the group consisting of aryl, arylalkoxy, arylalkyl, aryloxy, heterocycle, and heterocyclealkyl; and
R⁴ is other than alkenyl, alkoxyalkynyl, alkyl, alkynyl, cycloalkyl, aryl, arylalkyl, heterocycle, heterocyclealkyl, or —R_{C}R_{D}R_{E};
where R_{C} is selected from the group consisting of aryl, arylalkyl, heterocycle and heterocyclealkyl;
R_{D} is selected from the group consisting of aryl, arylalkoxy, arylalkoxylmino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxylmino, heterocycleoxylminoalkyl, and heterocyclesulfonyl; and
R_{E} is absent or selected from the group consisting of aryl, arylalkoxy, arylalkoxylmino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxylmino, heterocycleoxylminoalkyl, and heterocyclesulfonyl.

2. The compound according to claim 1, having the structure: 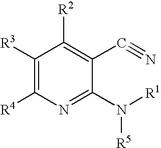
wherein:
R¹ is selected from the group consisting of —H, methyl, ethyl, propyl, butyl, —(CH₂)COOH, phenyl, pyridyl, dimethylaminoethyl, methoxyethyl, tetramethylaminoethyl, carboxymethyl, and phenylacetyl;
R² is selected from the group consisting of —H, methyl, ethyl, propyl, butyl, amino, phenyl, methoxy, carboxy, carboxymethyl, hydroxyethylamino, propylamino, ethylamino, methylamino, methoxyethyl, ethoxyethylamino, aminoethylamino, benzylamino, dimethylaminoethylamino, phthaloaminoethyl, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), tetramethylaminoethyl, acetylaminophenyl, methoxyphenylamino, carboxyphenyl, carboxy-3-isopyrryl, cyanophenyl, cyclopropyl, phenoxyphenyl, pyridyl, dihydroxybromophenyl, difluoromethoxyphenyl, trifluoromethylphenyl, trifluoromethylfluorophenyl, hydroxyphenyl, methylaminomethyl, methylaminoethyl, thiophyl, pyrryl, aminomethyl, 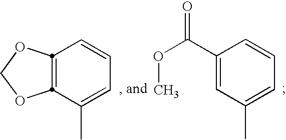
R³ is selected from the group consisting of —H, methyl, ethyl, propyl, isopropyl, cyano, aminomethyl, phenyl, fluorophenyl, and amino, except that when R² is pyrryl, R³ is other than cyano;
wherein the R² and R³ groups are such that they optionally join to form a ring system selected from: 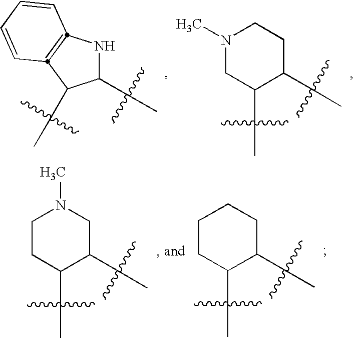
R⁴ is selected from the group consisting of —H, methyl, ethyl, propyl, hydroxy, furyl, methylfuryl, methylimidazolyl, phenyl, hydroxyphenyl, carboxyphenyl, pyrazolyl, hydroxy, dihydroxyphenyl, methoxyphenyl, chlorophenyl, bromophenyl, fluorophenyl, dichlorophenyl, dihydroxyborophenyl, thienyl, pyrryl, N-methylpyrryl, pyridyl, methylthio, methylsulfonylphenyl, carboethoxyphenyl, methoxy, carbamylphenyl, mercapto, N-isoimidazoylphenyl, isopropyl, amino, hydroxynaphthyl, thiazoyl, carboxymethylphenyl, trifluoromethylphenyl, methylphenyl, cyanophenyl, dimethylphenyl, fluorobenzhydryl, methoxyfuryl, aminosulfonylphenyl, 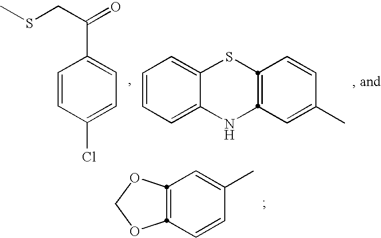
wherein the R³ and R⁴ groups are such that they optionally join to form a ring system selected from: 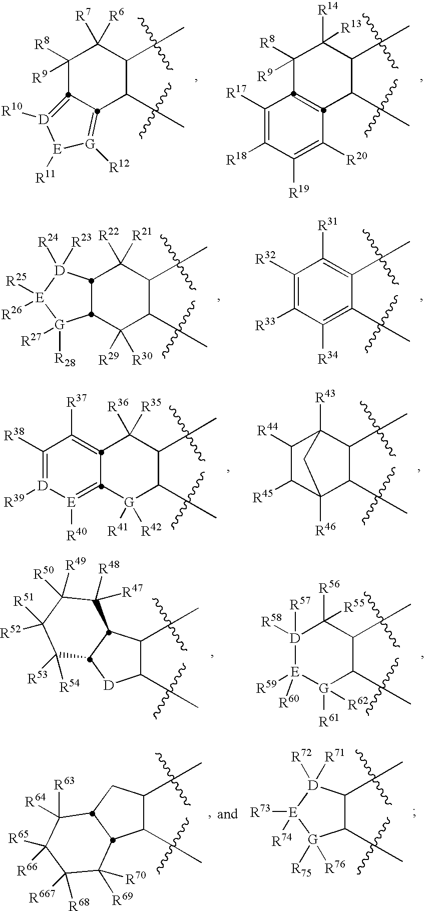
D, E and G are each independently selected from the group consisting of carbon, oxygen, sulfur, and nitrogen;
R⁵ is selected from the group consisting of —H, and C₁-C₅ alkyl, provided that at least one of R¹, R², R³, R⁴ and R⁵ is other than hydrogen; and
wherein the R¹ and R⁵ groups optionally join to form a piperidyl ring;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, and R⁷⁶ are each optionally present and are each independently selected from the group consisting of —H, methyl, ethyl, propyl, butyl, isobutyl, amino, nitro, hydroxy, methoxy, ethoxy, propoxy, 2-propenoxy, oxo, carboxy, bromo, chloro, fluoro, trifluoromethyl, chloromethyl, hydroxymethyl, dicyanomethyl, 2-fluorophenyl, 3-fluorophenyl, hydroxyethoxy, ethoxyethoxy, —(CH₂)—O—(C₆H₄)—O—(CH₃), carboxymethoxy, isopropylcarboxymethoxy, isobutylcarboxymethoxy, methylamino, dimethylamino, aminoethoxy, diaminoethoxy, dimethylaminoethoxy, cyanomethoxymethyl, 2-propenoxymethyl, methoxymethyl, isopropoxymethyl, ethoxymethyl, —(CH₂)—O—(CF₂)—CHF₂, isobutoxymethyl, benzoyl, phenyl, N-morpholinyl, morpholinylethoxy, pyrrolidylethoxy, N-pyrrolidylethoxy, oxo, ethylcarboxy, carboxymethyl-ethyl ester, pyridylmethyl, 4-pyridylmethoxy, 2-pyridylmethyl, and —COO—CH₂—CH₃, with the proviso that when G is —N—, R³⁶ is —H; and
wherein R³⁸ and R³⁹ are such that they optionally join to form a ring system of the type selected from:

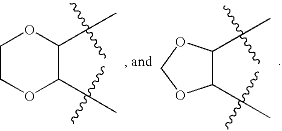

3. The compound according to claim 2, wherein:
R¹ is selected from the group consisting of —H, methyl, ethyl, —(CH₂)COOH, and phenyl;
R² is selected from the group consisting of —H, methyl, ethyl, amino, phenyl, methoxy, carboxy, hydroxyethylamino, propylamino, ethylamino, methylamino, methoxyethyl, ethoxyethylamino, aminoethylamino, benzylamino, dimethylaminoethylamino, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), acetylaminophenyl, methoxyphenylamino, carboxyphenyl, cyanophenyl, cyclopropyl, phenoxyphenyl, pyridyl, dihydroxybromophenyl, difluoromethoxyphenyl, trifluoromethylphenyl, trifluoromethylfluorophenyl, hydroxyphenyl, 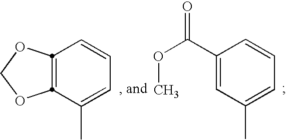
R³ is selected from the group consisting of —H, methyl, ethyl, propyl, isopropyl, cyano, and aminomethyl;
wherein the R² and R³ groups are such that they optionally join to form a ring system selected from: 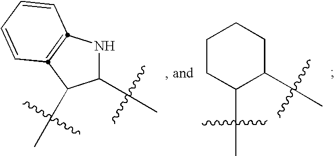
R⁴ is selected from the group consisting of —H, methyl, ethyl, propyl, hydroxy, furyl, indolyl, methylfuryl, methylimidazolyl, phenyl, hydroxyphenyl, carboxyphenyl, pyrazolyl, hydroxy, dihydroxyphenyl, methoxyphenyl, chlorophenyl, dichlorophenyl, dihydroxyborophenyl, thienyl, pyrryl, N-methylpyrryl, pyridyl, methylthio, methylsulfonylphenyl, carboethoxyphenyl, methoxy, carbamylphenyl, N-isoimidazoylphenyl, amino, hydroxynaphthyl, thiazoyl, carboxymethylphenyl, aminosulfonylphenyl, and 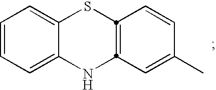
wherein the R³ and R⁴ groups are such that they can join to form a ring system selected from: 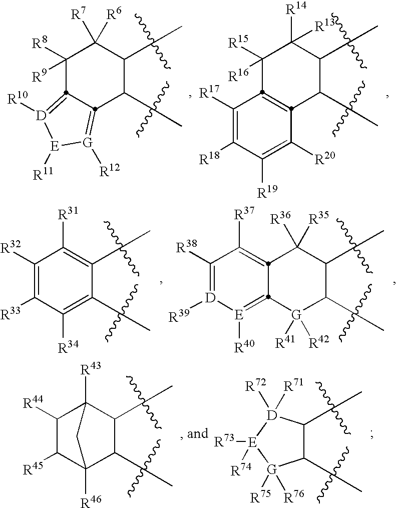
D, E and G are each independently selected from the group consisting of carbon, oxygen, sulfur, and nitrogen;
R⁵ is selected from the group consisting of —H, and C₁-C₅ alkyl, provided that at least one of R¹, R², R³, R⁴ and R⁵ is other than hydrogen;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁷¹, R⁷², R⁷³, R⁷⁴, R⁷⁵, and R⁷⁶ are each optionally present and are each independently selected from the group consisting of —H, methyl, ethyl, butyl, amino, nitro, hydroxy, methoxy, ethoxy, oxo, 2-propenoxy, carboxy, bromo, chloro, fluoro, trifluoromethyl, chloromethyl, hydroxymethyl, dicyanomethyl, hydroxyethoxy, ethoxyethoxy, —(CH₂)—O—(C₆H₄)—O—(CH₃), carboxymethoxy, isopropylcarboxymethoxy, methylamino, dimethylamino, aminoethoxy, diaminoethoxy, cyanomethoxymethyl, methoxymethyl, isopropoxymethyl, ethoxymethyl, —(CH₂)—O—(CF₂)—CHF₂, isobutoxymethyl, phenyl, morpholinylethoxy, pyrrolidylethoxy, N-pyrrolidylethoxy, and pyridylmethyl, with the proviso that when G is —N—, R³⁶ is —H; and
wherein R³⁸ and R³⁹ are such that they optionally join to form a ring system of the type selected from:

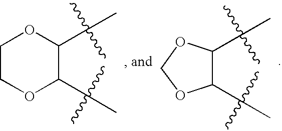

4. The compound according to claim 2, wherein:
R¹ is selected from the group consisting of —H, methyl, and ethyl;
R² is selected from the group consisting of —H, methyl, amino, phenyl, methoxy, hydroxyethylamino, propylamino, ethylamino, methylamino, methoxyethyl, ethoxyethylamino, aminoethylamino, benzylamino, dimethylaminoethylamino, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), acetylaminophenyl, methoxyphenylamino, carboxyphenyl, cyanophenyl, cyclopropyl, phenoxyphenyl, pyridyl, dihydroxybromophenyl, difluoromethoxyphenyl, and 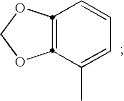
R³ is selected from the group consisting of —H, methyl, ethyl, propyl, isopropyl, and cyano;
wherein the R² and R³ groups are such that they optionally join to form a ring system selected from: 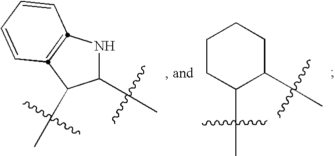
R⁴ is selected from the group consisting of —H, methyl, ethyl, propyl, hydroxy, furyl, indolyl, methylfuryl, methylimidazolyl, phenyl, hydroxyphenyl, carboxyphenyl, pyrazolyl, hydroxy, dihydroxyphenyl, methoxyphenyl, chlorophenyl, dichlorophenyl, dihydroxyborophenyl, thienyl, pyrryl, N-methylpyrryl, pyridyl, methylthio, methylsulfonylphenyl, carboethoxyphenyl, methoxy, carbamylphenyl, amino, and aminosulfonylphenyl;
wherein the R³ and R⁴ groups are such that they optionally join to form a ring system selected from: 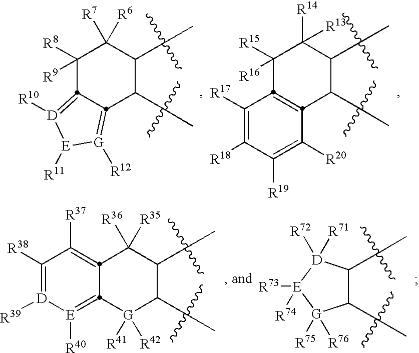
D, E and G are each independently selected from the group consisting of carbon, oxygen, sulfur, and nitrogen;
R⁵ is —H, provided that at least one of R¹, R², R³, R⁴ and R⁵ is other than hydrogen;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R³⁵, R³⁶, R³⁷, R³⁸, R³⁹, R⁴⁰, R⁴¹, R⁴², R⁷¹, R⁷², R⁷³, R⁷⁴, R⁷⁵, and R⁷⁶ are each optionally present and are each independently selected from the group consisting of —H, methyl, ethyl, butyl, amino, nitro, hydroxy, methoxy, ethoxy, oxo, 2-propenoxy, carboxy, bromo, fluoro, trifluoromethyl, chloromethyl, dicyanomethyl, hydroxyethoxy, ethoxyethoxy, —(CH₂)—O—(C₆H₄)—O—(CH₃), carboxymethoxy, isopropylcarboxymethoxy, methylamino, dimethylamino, aminoethoxy, diaminoethoxy, phenyl, morpholinylethoxy, pyrrolidylethoxy, N-pyrrolidylethoxy, and pyridylmethyl, with the proviso that when G is —N—, R³⁶ is —H; and
wherein R³⁸ and R³⁹ are such that they can join to form a ring system consisting of:

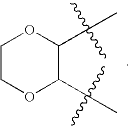

5. The compound according to claim 2, wherein:
R¹ is selected from the group consisting of —H, methyl, and ethyl;
R² is selected from the group consisting of —H, methyl, amino, phenyl, methoxy, hydroxyethylamino, propylamino, ethylamino, methylamino, methoxyethyl, ethoxyethylamino, aminoethylamino, benzylamino, dimethylaminoethylamino, fluorophenyl, difluorophenyl, chlorophenyl, bromophenyl, furyl, carbamylpyrryl, methyl-1,3-isodiazoyl, 1,3-isodiazoyl, 1,3,4-triazoyl, methoxyphenyl, —S(CH₃), acetylaminophenyl, methoxyphenylamino, carboxyphenyl, and 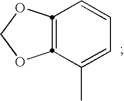
R³ is selected from the group consisting of —H, methyl, ethyl, propyl, and isopropyl;
wherein the R² and R³ groups are optionally such that they join to form: 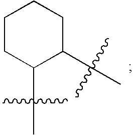
R⁴ is selected from the group consisting of —H, methyl, ethyl, propyl, furyl, indolyl, methylfuryl, methylimidazolyl, phenyl, hydroxyphenyl, carboxyphenyl, pyrazolyl, hydroxy, dihydroxyphenyl, methoxyphenyl, chlorophenyl, dichlorophenyl, dihydroxyborophenyl, thienyl, pyrryl, N-methylpyrryl, pyridyl, methylthio, methylsulfonylphenyl, carboethoxyphenyl, and aminosulfonylphenyl;
wherein the R³ and R⁴ groups are such that they optionally join to form a ring system selected from: 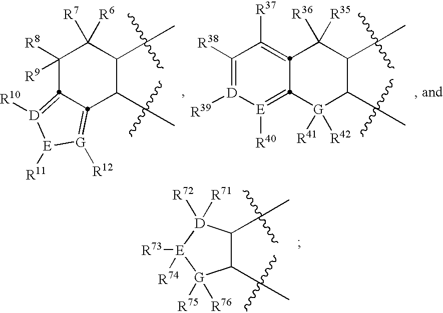
D, E and G are each independently selected from the group consisting of carbon, oxygen, sulfur, and nitrogen;
R⁵ is —H, provided that at least one of R¹, R², R³, R⁴ and R⁵ is other than hydrogen;
R⁶, R⁷, R⁸, R⁹, R¹⁰, R¹¹, R¹², R³⁵, R³⁶, R³⁷, R³⁸, R³⁹, R⁴⁰, R⁴¹, R⁴², R⁷¹, R⁷², R⁷³, R⁷⁴, R⁷⁵, and R⁷⁶ are each optionally present and are each independently selected from the group consisting of —H, methyl, ethyl, butyl, amino, nitro, hydroxy, methoxy, ethoxy, oxo, 2-propenoxy, carboxy, bromo, fluoro, trifluoromethyl, chloromethyl, dicyanomethyl, hydroxyethoxy, ethoxyethoxy, carboxymethoxy, isopropylcarboxymethoxy, methylamino, dimethylamino, aminoethoxy, diaminoethoxy, morpholinylethoxy, pyrrolidylethoxy, N-pyrrolidylethoxy, and pyridylmethyl, with the proviso that when G is —N—, R³⁶ is —H; and
wherein R³⁸ and R³⁹ are such that they optionally join to form a ring system consisting of:

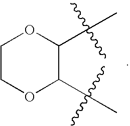

6. The compound according to claim 2, wherein:
R¹ is —H;
R² is selected from the group consisting of amino, phenyl, fluorophenyl, difluorophenyl, furyl, carbamylpyrryl, methyl-1,3-isodiazoyl, 1,3-isodiazoyl, 1,3,4-triazoyl, methoxyphenyl, acetylaminophenyl, methoxyphenylamino, and carboxyphenyl;
R³ is selected from the group consisting of —H, methyl, ethyl, and propyl;
R⁴ is selected from the group consisting of methyl, ethyl, propyl, furyl, phenyl, hydroxyphenyl, carboxyphenyl, pyrazolyl, hydroxy, dihydroxyphenyl, methoxyphenyl, chlorophenyl, dihydroxyborophenyl, and aminosulfonylphenyl;
wherein the R³ and R⁴ groups are such that they optionally join to form a ring system selected from: 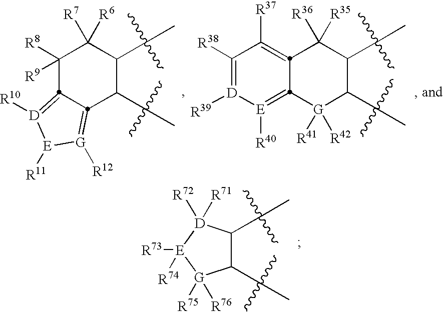
D, E and G are each independently selected from the group consisting of carbon, oxygen, sulfur, and nitrogen;
R⁵ is —H, provided that at least one of R¹, R², R³, R⁴ and R⁵ is other than hydrogen;
R⁶, R⁷, R⁸, R⁹, R¹⁰, R¹¹, R¹², R³⁵, R³⁶, R³⁷, R³⁸, R³⁹, R⁴⁰, R⁴¹, R⁴², R⁷¹, R⁷², R⁷³, R⁷⁴, R⁷⁵, and R⁷⁶ are each optionally present and are each independently selected from the group consisting of —H, amino, nitro, hydroxy, methoxy, ethoxy, oxo, 2-propenoxy, carboxy, bromo, fluoro, trifluoromethyl, chloromethyl, dicyanomethyl, hydroxyethoxy, ethoxyethoxy, carboxymethoxy, isopropylcarboxymethoxy, methylamino, dimethylamino, aminoethoxy, diaminoethoxy, morpholinylethoxy, pyrrolidylethoxy, and pyridylmethyl, with the proviso that when G is —N—, R³⁶ is —H; and
wherein R³⁸ and R³⁹ optionally are such that they optionally join to form:

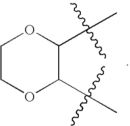

7. The compound according to claim 2, wherein the aminocyanopyridine MK-2 inhibiting compound comprises at least one compound that is selected from the group consisting of:
2-amino-4-(2-fluorophenyl)-6,8-dihydro-5H-pyrazolo[3,4-h]quinoline-3-carbonitrile,
2-amino-4-(2-furyl)-6,7-dihydro-5H-pyrazolo[3,4-h]quinoline-3-carbonitrile,
2-amino-4-(2,3-difluorophenyl)-6,7-dihydro-5H-pyrazolo[3,4-h]quinoline-3-carbonitrile,
8-amino-6-(2-furyl)-4,5-dihydro-1H-pyrazolo[4,3-h]quinoline-7-carbonitrile,
2-amino-3-cyano-4-(2-furyl)-5,6-dihydrobenzo[h]quinoline-8-carboxylic acid,
4-[2-amino-3-cyano-6-(2-furyl)pyridin-4-yl]-1H-pyrrole-2-carboxamide,
2-amino-4-phenyl-6,8-dihydro-5H-pyrazolo[3,4-h]quinoline-3-carbonitrile,
2-amino-6-(2-furyl)-4-(1-methyl-1H-imidazol-4-yl)nicotinonitrile,
8-amino-6-(2-furyl)-4,5-dihydro-1H-pyrazolo[4,3-h]quinoline-7-carbonitrile,
2-amino-4-(2-furyl)-8-hydroxy-5,6-dihydrobenzo[h]quinoline-3-carbonitrile,
2-amino-4-(2,6-difluorophenyl)-6,7-dihydro-5H-pyrazolo[3,4-h]quinoline-3-carbonitrile,
2-amino-6-(4-hydroxyphenyl)-4-(1H-imidazol-5-yl)nicotinonitrile,
2-amino-4-(2-fluorophenyl)-6-(2-furyl)nicotinonitrile, 2-amino-4-(2-fluorophenyl)-6-(2-furyl)nicotinonitrile,
2-amino-4-(2-fluorophenyl)-5,6-dihydrobenzo[h]quinoline-3-carbonitrile,
4-[6-amino-5-cyano-4-(2-furyl)pyridin-2-yl]benzoic acid,
2-amino-6-(2-furyl)-4-(1H-imidazol-5-yl)nicotinonitrile,
2-amino-4-(2-furyl)-6-(1H-pyrazol-3-yl)nicotinonitrile,
2-amino-3-cyano-4-(4H-1,2,4-triazol-3-yl)-5,6-dihydrobenzo[h]quinoline-8-carboxylic acid,
2-amino-6-(3-hydroxyphenyl)-4-(1H-imidazol-5-yl)nicotinonitrile,
2-amino-6-(2-furyl)-4-(1H-imidazol-4-yl)nicotinonitrile,
2-amino-4-(2,4-difluorophenyl)-6,7-dihydro-5H-pyrazolo[3,4-h]quinoline-3-carbonitrile,
4,6-diamino-2-(trifluoromethyl)-2,3-dihydrofuro[2,3-b]pyridine-5-carbonitrile,
2-amino-4-(2-furyl)-6,8-dihydro-5H-pyrrolo[3,4-h]quinoline-3-carbonitrile,
4-[6-amino-5-cyano-4-(2-fluorophenyl)pyridin-2-yl]benzoic acid,
2-amino-4-(2-furyl)-5,6-dihydro-1,8-phenanthroline-3-carbonitrile,
2-amino-6-(3,4-dihydroxyphenyl)-4-(2-fluorophenyl)nicotinonitrile,
2-amino-4-(1-methyl-1H-imidazol-4-yl)-6-phenylnicotinonitrile,
2-amino-4-(2-furyl)-6-(1H-pyrazol-3-yl)nicotinonitrile,
4-[6-amino-5-cyano-4-(1H-imidazol-5-yl)pyridin-2-yl]benzoic acid,
2-amino-4-(3-fluorophenyl)-6,8-dihydro-5H-pyrazolo[3,4-h]quinoline-3-carbonitrile,
2-amino-6-(3,4-dihydroxyphenyl)-4-(2-fluorophenyl)nicotinonitrile,
N-{4-[6-amino-5-cyano-4-(2-furyl)pyridin-2-yl]phenyl}methanesulfonamide,
2-amino-4-(2-furyl)-6,7-dihydro-5H-pyrrolo[2,3-h]quinoline-3-carbonitrile,
2-amino-4-(1H-imidazol-5-yl)-6-phenylnicotinonitrile,
2-amino-4-(2-furyl)-5,6-dihydrobenzo[h]quinoline-3-carbonitrile,
2-amino-4-(1H-imidazol-5-yl)-6-(4-methoxyphenyl)nicotinonitrile,
2-amino-6-(3-chlorophenyl)-4-(1H-imidazol-5-yl)nicotinonitrile,
2-amino-4-(2-furyl)-6-(1H-pyrazol-4-yl)nicotinonitrile,
2-amino-4-(4-methoxyphenyl)-6,7-dihydro-5H-pyrazolo[3,4-h]quinoline-3-carbonitrile,
2-amino-4-(2,5-difluorophenyl)-6,7-dihydro-5H-pyrazolo[3,4-h]quinoline-3-carbonitrile,
2-amino-4-(4-fluorophenyl)-6,8-dihydro-5H-pyrazolo[3,4-h]quinoline-3-carbonitrile,
2-amino-4-(4H-1,2,4-triazol-3-yl)-5,6-dihydrobenzo[h]quinoline-3-carbonitrile,
4,6-diamino-2-(chloromethyl)-2,3-dihydrofuro[2,3-b]pyridine-5-carbonitrile,
2-amino-4-(1H-imidazol-4-yl)-6-phenylnicotinonitrile,
4-[6-amino-5-cyano-4-(2-furyl)pyridin-2-yl]benzenesulfonamide,
4-[6-amino-5-cyano-4-(2-furyl)pyridin-2-yl]phenylboronic acid,
2-amino-6-(4-methoxyphenyl)-4-(4H-1,2,4-triazol-3-yl)nicotinonitrile,
2-amino-4-(2-fluorophenyl)-6-(3-furyl)nicotinonitrile,
2-amino-6-(2-furyl)-4-(methylthio)nicotinonitrile,
2-amino-4-(2-fluorophenyl)-6-(3-hydroxyphenyl)nicotinonitrile,
8-amino-6-(2-furyl)-4,5-dihydro-2H-pyrazolo[4,3-h]quinoline-7-carbonitrile,
2-amino-4-(2-bromophenyl)-6-(2-furyl)nicotinonitrile,
2-amino-4-(2-fluorophenyl)-6-(4-hydroxyphenyl)nicotinonitrile,
2-amino-4-phenyl-6-thien-2-ylnicotinonitrile,
2-amino-4-(3-methoxyphenyl)-6,7-dihydro-5H-pyrazolo[3,4-h]quinoline-3-carbonitrile,
2-amino-4-(2-furyl)-7-methyl-6,7-dihydro-5H-pyrazolo[3,4-h]quinoline-3-carbonitrile,
2-amino-4-(2-fluorophenyl)-6-(1H-pyrrol-2-yl)nicotinonitrile,
2-amino-4-(2-furyl)-5-methyl-6,8-dihydro-5H-pyrazolo[3,4-h]quinoline-3-carbonitrile,
2-amino-4-(2-furyl)-6-(1-methyl-1H-pyrrol-3-yl)nicotinonitrile,
3-amino-5,6,7,8-tetrahydroisoquinoline-4-carbonitrile,
N-[4-(2-amino-3-cyano-6,7-dihydro-5H-pyrazolo[3,4-h]quinolin-4-yl)phenyl]acetamide,
6-amino-4-[(4-methoxyphenyl)amino]-2-(trifluoromethyl)-2,3-dihydrofuro[2 ,3-b]pyridine-5-carbonitrile,
4-[6-amino-5-cyano-4-(2-furyl)pyridin-2-yl]-N-(tert-butyl)benzenesulfonamide,
4,6-diamino-2-ethyl-2,3-dihydrofuro[2,3-b]pyridine-5-carbonitrile,
6-amino-4-(2-furyl)-2,4′-bipyridine-5-carbonitrile,
2,4-diamino-6-(methylthio)nicotinonitrile,
3-(2-amino-3-cyano-6,7-dihydro-5H-pyrazolo[3,4-h]quinolin-4-yl)benzoic acid,
2-amino-6-(4-chlorophenyl)-4-(1H-imidazol-5-yl)nicotinonitrile,
2-amino-4-(1,3-benzodioxol-4-yl)-6,7-dihydro-5H-pyrazolo[3,4-h]quinoline-3-carbonitrile,
4,6-diamino-2-methyl-2,3-dihydrofuro[2,3-b]pyridine-5-carbonitrile,
2-amino-4-(1H-imidazol-5-yl)-6-[4-(methylsulfonyl)phenyl]nicotinonitrile,
2,4-diaminoquinoline-3-carbonitrile,
2,8-diamino-4-(2-furyl)-5,6-dihydrobenzo[h]quinoline-3-carbonitrile,
2-amino-4,6-di(2-furyl)nicotinonitrile,
4,6-diamino-2-butyl-2,3-dihydrofuro[2,3-b]pyridine-5-carbonitrile,
ethyl 4-[6-amino-5-cyano-4-(1H-imidazol-5-yl)pyridin-2-yl]benzoate,
2,4-diamino-6-methoxynicotinonitrile,
2-amino-4-methylnicotinonitrile,
2-amino-4-(4-cyanophenyl)-6,7-dihydro-5H-pyrazolo[3,4-h]quinoline-3-carbonitrile,
2-amino-4-cyclopropyl-6-methylnicotinonitrile,
2-amino-4-(2-furyl)-6-(1-methyl-1H-pyrrol-2-yl)nicotinonitrile,
2-amino-4-(2-chlorophenyl)-6,7-dihydro-5H-pyrazolo[3,4-h]quinoline-3-carbonitrile,
2-amino-6-(2-furyl)-4-(4-phenoxyphenyl)nicotinonitrile,
2-amino-4-pyridin-3-yl-6,8-dihydro-5H-pyrazolo[3,4-h]quinoline-3-carbonitrile,
2-amino-6-{[2-(4-chlorophenyl)-2-oxoethyl]thio}-4-(2-furyl)pyridine-3,5-dicarbonitrile,
4-[2-amino-3-cyano-6-(2-furyl)pyridin-4-yl]phenylboronic acid,
2-amino-6-(3-chlorophenyl)-4-(1H-imidazol-4-yl)nicotinonitrile,
4-(6-amino-5-cyano-4-phenylpyridin-2-yl)-N-(tert-butyl)benzenesulfonamide,
2-amino-4-methoxynicotinonitrile,
4-[2-amino-3-cyano-6-(2-furyl)pyridin-4-yl]benzoic acid,
4,6-diamino-2-[(4-methoxyphenoxy)methyl]-2,3-dihydrofuro[2,3-b]pyridine-5-carbonitrile,
2-amino-4-(2-fluorophenyl)-6-(4-methoxyphenyl)nicotinonitrile,
4-[6-amino-5-cyano-4-(2-fluorophenyl)pyridin-2-yl]-N-(tert-butyl)benzenesulfonamide,
(2,4-diamino-3-cyano-5H-chromeno[2,3-b]pyridin-9-yl)oxy]acetic acid,
3-Pyridinecarbonitrile, 2-Amino-4-Methylm
2-amino-6-(2-furyl)nicotinonitrile,
2-amino-4-(2-furyl)-6-(3-hydroxyphenyl)nicotinonitrile,
4-[6-amino-5-cyano-4-(2-furyl)pyridin-2-yl]benzamide,
2-amino-4-(2-furyl)-7-hydroxy-5,6-dihydrobenzo[h]quinoline-3-carbonitrile,
2-amino-4-(2-furyl)-6-(1H-indol-3-yl)nicotinonitrile,
2-amino-4-pyridin-4-yl-6,8-dihydro-5H-pyrazolo[3,4-h]quinoline-3-carbonitrile,
2-amino-4-(3-fluorophenyl)-6-(4-hydroxyphenyl)nicotinonitrile,
2-amino-4-[2-(difluoromethoxy)phenyl]-6,7-dihydro-5H-pyrazolo[3,4-h]quinoline-3-carbonitrile,
2-amino-4-(2-furyl)-6-thien-3-ylnicotinonitrile,
2-amino-4-(3-fluorophenyl)-6-(4-methoxyphenyl)nicotinonitrile,
2-[2-amino-3-cyano-6-(2-furyl)pyridin-4-yl]phenylboronic acid,
2,4-diamino-6-propylpyridine-3,5-dicarbonitrile,
4,6-diamino-2-[(prop-2-ynyloxy)methyl]-2,3-dihydrofuro[2,3-b]pyridine-5-carbonitrile,
4,6-diamino-2-(hydroxymethyl)-2,3-dihydrofuro[2,3-b]pyridine-5-carbonitrile,
2-amino-6-(2-furyl)-4-[4-(trifluoromethyl)phenyl]nicotinonitrile,
5-amino-7-methylthieno[3,2-b]pyridine-6-carbonitrile,
2-amino-4-(2-furyl)-5,5-dimethyl-6,8-dihydro-5H-pyrazolo[3,4-h]quinoline-3-carbonitrile,
N-[3-cyano-4-(2-fluorophenyl)-6-(2-furyl)pyridin-2-yl]glycine,
2-[(allyloxy)methyl]-4,6-diamino-2,3-dihydrofuro[2,3-b]pyridine-5-carbonitrile,
2-amino-4-(2-furyl)-6-methyl-5,6-dihydrobenzo[h]quinoline-3-carbonitrile,
4,6-diamino-2-(methoxymethyl)-2,3-dihydrofuro[2,3-b]pyridine-5-carbonitrile,
2-amino-4-(2-furyl)-6-(1H-indol-3-yl)nicotinonitrile,
2-amino-4-(2-furyl)-6-[4-(1H-imidazol-1-yl)phenyl]nicotinonitrile,
2-amino-4-(2-furyl)-6-(4-hydroxyphenyl)nicotinonitrile,
2-amino-4-(2-furyl)-5,6,7,8-tetrahydro-5,8-methanoquinoline-3-carbonitrile,
4,6-diamino-2-(isopropoxymethyl)-2,3-dihydrofuro[2,3-b]pyridine-5-carbonitrile,
3-[6-amino-5-cyano-4-(2-furyl)pyridin-2-yl]phenylboronic acid,
4,6-diamino-2-(ethoxymethyl)-2,3-dihydrofuro[2,3-b]pyridine-5-carbonitrile,
2-amino-4-(4-bromophenyl)-6-(2-furyl)nicotinonitrile,
4,6-diamino-2-[(1,1,2,2-tetrafluoroethoxy)methyl]-2,3-dihydrofuro[2,3-b]pyridine-5-carbonitrile,
2-amino-4-[2-fluoro-4-(trifluoromethyl)phenyl]-6-(2-furyl)nicotinonitrile,
2-amino-4-(2-methoxyphenyl)-6,8-dihydro-5H-pyrazolo[3,4-h]quinoline-3-carbonitrile,
2-amino-4-(2-fluorophenyl)-5-methyl-6,8-dihydro-5H-pyrazolo[3,4-h]quinoline-3-carbonitrile,
3,6-diamino-4-ethyl-1H-pyrazolo[3,4-b]pyridine-5-carbonitrile,
6-amino-4-(2-furyl)-2,2′-bipyridine-5-carbonitrile,
2-amino-4-(2-furyl)-6-(8-hydroxy-1-naphthyl)nicotinonitrile,
4-(2-amino-3-cyano-6,7-dihydro-5H-pyrazolo[3,4-h]quinolin-4-yl)benzoic acid,
2-amino-6-(3,4-dichlorophenyl)-4-(2-furyl)nicotinonitrile,
2-amino-4-(2-furyl)-6-(10H-phenothiazin-2-yl)nicotinonitrile,
sodium 2-amino-3-cyano-4-quinolinecarboxylate,
2-anilino-4-(2-fluorophenyl)-6-(2-furyl)nicotinonitrile,
2-amino-4-(3-fluorophenyl)-6-(2-furyl)nicotinonitrile,
2-amino-4-(4-fluorophenyl)-6-(2-furyl)nicotinonitrile,
4,6-diamino-2-(tert-butoxymethyl)-2,3-dihydrofuro[2,3-b]pyridine-5-carbonitrile,
2-amino-4-(2-furyl)-6-(1,3-thiazol-2-yl)nicotinonitrile,
4-(2-fluorophenyl)-6-(2-furyl)-2-piperidin-1-ylnicotinonitrile,
2-amino-6-(4-chlorophenyl)-4-(2-furyl)nicotinonitrile,
2-amino-6-(4-hydroxyphenyl)-4-(2-methoxyphenyl)nicotinonitrile,
2-amino-6-(2-furyl)-4-(2-hydroxyphenyl)nicotinonitrile,
methyl 3-(2-amino-3-cyano-6,7-dihydro-5H-pyrazolo[3,4-h]quinolin-4-yl)benzoate,
2-amino-4-(2-chlorophenyl)-6-(5-methyl-2-furyl)nicotinonitrile,
3,6-diamino-2-benzoylthieno[2,3-b]pyridine-5-carbonitrile,
methyl 4-[6-amino-5-cyano-4-(2-furyl)pyridin-2-yl]benzoate,
2-aminonicotinonitrile,
2-amino-4-(2-furyl)-8-{[2-(trimethylsilyl)ethoxy]methyl}-6,8-dihydro-5H-pyrazolo[3,4-h]quinoline-3-carbonitrile,
3-amino-5H-pyrido[4,3-b]indole-4-carbonitrile,
2-(2-amino-3-cyano-6,7-dihydro-5H-pyrazolo[3,4-h]quinolin-4-yl)benzoic acid,
2-amino-6-(4-methoxyphenyl)-4-phenylnicotinonitrile,
2-amino-4-(2-furyl)-5,6,7,8-tetrahydroquinoline-3-carbonitrile,
2-amino-4-(2-furyl)-6-isobutylnicotinonitrile,
2-amino-6-benzyl-4-(2-furyl)nicotinonitrile,
2-amino-4-(2-furyl)-6-methyl-5-phenylnicotinonitrile,
2-amino-4-(2-furyl)-6-[4-(trifluoromethoxy)phenyl]nicotinonitrile,
2-amino-4-(2-furyl)-6-propyl-5,6,7,8-tetrahydro-1,6-naphthyridine-3-carbonitrile,
2-amino-4-(2-furyl)benzo[h]quinoline-3-carbonitrile,
2-amino-6-(4-methoxyphenyl)-4-thien-2-ylnicotinonitrile,
2-amino-4-(2-fluorophenyl)-6-tetrahydrofuran-2-ylnicotinonitrile,
ethyl 6-amino-5-cyano-4-(2-furyl)pyridine-2-carboxylate,
2-amino-4-(2-furyl)-9-methoxy-5,6-dihydrobenzo[h]quinoline-3-carbonitrile,
2-amino-4-(2-furyl)-8-methoxy-5,6-dihydrobenzo[h]quinoline-3-carbonitrile,
2-amino-4-(2-furyl)-8,9-dimethoxy-5,6-dihydrobenzo[h]quinoline-3-carbonitrile,
2-amino-4-(2-furyl)-7-methoxy-5,6-dihydrobenzo[h]quinoline-3-carbonitrile,
2-amino-4-(2-furyl)-7,9-dimethyl-5,6-dihydrobenzo[h]quinoline-3-carbonitrile,
ethyl 4-[6-amino-5-cyano-4-(2-furyl)pyridin-2-yl]benzoate,
2-amino-6-(3-bromophenyl)-4-(2-furyl)nicotinonitrile,
2-amino-4-(2-furyl)-6-[4-(trifluoromethyl)phenyl]nicotinonitrile,
2-amino-4-(2-furyl)-6-[3-(trifluoromethyl)phenyl]nicotinonitrile,
2-amino-4-(2-furyl)-6-[4-(methylsulfonyl)phenyl]nicotinonitrile,
4,6-diamino-2-(phenoxymethyl)-2,3-dihydrofuro[2,3-b]pyridine-5-carbonitrile,
4,6-diamino-3-phenyl-2,3-dihydrofuro[2,3-b]pyridine-5-carbonitrile,
4,6-diamino-3-vinyl-2,3-dihydrofuro[2,3-b]pyridine-5-carbonitrile,
2-amino-4-(2-fluorophenyl)-5-methyl-6,8-dihydro-5H-pyrazolo[3,4-h]quinoline-3-carbonitrile,
3-amino-1-methyl-5,6,7,8-tetrahydroisoquinoline-4-carbonitrile,
2-amino-4-(2-fluorophenyl)-5,5-dimethyl-6,8-dihydro-5H-pyrazolo[3,4-h]quinoline-3-carbonitrile,
2-amino-4-(2-fluorophenyl)-6-(3-hydroxyphenyl)nicotinonitrile,
2-amino-4-[2-(difluoromethoxy)phenyl]-6,7-dihydro-5H-pyrazolo[3,4-h]quinoline-3-carbonitrile,
2-(benzylamino)-4-(2-fluorophenyl)-6-(2-furyl)nicotinonitrile,
2-amino-4-(2-furyl)-6,7-dihydro-5H-benzo[6,7]cyclohepta[1,2-b]pyridine-3-carbonitrile,
2-amino-4-(2-furyl)-5H-indeno[1,2-b]pyridine-3-carbonitrile,
3-amino-1-methyl-5,6,7,8-tetrahydroisoquinoline-4-carbonitrile,
2-amino-4-(2-fluorophenyl)-6-(3-hydroxyphenyl)nicotinonitrile,
2-amino-4-(2-thienyl)-5,6,7,8-tetrahydro-3-quinolinecarbonitrile,
2-amino-4-(3-fluorophenyl)-5,6,7,8-tetrahydro-3-quinolinecarbonitrile,
2-(1-piperidinyl)-6-(2-thienyl)-4-(trifluoromethyl)nicotinonitrile,
2-(dimethylamino)-6-(2-thienyl)-4-(trifluoromethyl)nicotinonitrile,
3-Quinolinecarbonitrile,
2-amino-4-methyl- or 2-amino-4-methyl-3-quinolinecarbonitrile,
2-amino-4-(4-methoxyphenyl)-6-(2-thienyl)nicotinonitrile,
2-amino-6-cyclopropyl-4-(2-methoxyphenyl)nicotinonitrile,
2-amino-4-(2-fluorophenyl)-6-phenylnicotinonitrile,
(4bS,8aR)-2,4-diamino-4b,5,6,7,8,8a-hexahydro[1]benzofuro[2,3-b]pyridine-3-carbonitrile,
2-amino-4-(2-fluorophenyl)-5,5-dimethyl-6,8-dihydro-5H-pyrazolo[3,4-h]quinoline-3-carbonitrile,
2-amino-4-(2-furyl)-5-phenyl-6,8-dihydro-5H-pyrazolo[3,4-h]qu inoline-3-carbonitrile,
3-amino-1,6-dimethyl-5,6,7,8-tetrahydro-2,6-naphthyridine-4-carbonitrile,
3-amino-1,7-dimethyl-5,6,7,8-tetrahydro-2,7-naphthyridine-4-carbonitrile,
2-amino-4-(2-fluorophenyl)-5-phenyl-6,8-dihydro-5H-pyrazolo[3,4-h]quinoline-3-carbonitrile,
2-amino-4-(2-fluorophenyl)-5-phenyl-6,8-dihydro-5H-pyrazolo[3,4-h]quinoline-3-carbonitrile,
4,6-diamino-2-(morpholin-4-ylmethyl)-2,3-dihydrofuro[2,3-b]pyridine-5-carbonitrile,
ethyl (4,6-diamino-5-cyano-2-oxo-2,3-dihydro-1H-pyrrolo[2,3-b]pyridin-1-yl)acetate,
2-amino-4-(2-methoxyphenyl)-6-(5-methyl-2-furyl)nicotinonitrile,
2-amino-6-methyl-4-(4-nitrophenyl)nicotinonitrile,
2-amino-4-(3,4-dimethoxyphenyl)-6-(5-methyl-2-furyl)nicotinonitrile,
2,4-diamino-6-[(4-methoxyphenyl)thio]nicotinonitrile,
4,6-diamino-2-(phenoxymethyl)-2,3-dihydrofuro[2,3-b]pyridine-5-carbonitrile,
4,6-diamino-3-phenyl-2,3-dihydrofuro[2,3-b]pyridine-5-carbonitrile,
4,6-diamino-2-[(2-methylphenoxy)methyl]-2,3-dihydrofuro[2,3-b]pyridine-5-carbonitrile,
2-amino-4-(2-furyl)-6-(4-methoxyphenyl)nicotinonitrile,
2-amino-4-(3-fluorophenyl)-5,6-dihydrobenzo[h]quinoline-3-carbonitrile,
2-amino-4-(4-methoxyphenyl)-6,7-dihydro-5H-cyclopenta[b]pyridine-3-carbonitrile,
2-amino-9-ethyl-9H-pyrido[2,3-b]indole-3-carbonitrile,
2-amino-6-isobutyl-4-(4-methylphenyl)nicotinonitrile,
1-(2-furyl)-3-[(3-hydroxypropyl)amino]-5,6,7,8-tetrahydroisoquinoline-4-carbonitrile,
2-azepan-1-yl-6-(4-fluorophenyl)-4-phenylnicotinonitrile,
2-amino-6-tert-butyl-4-(4-methylphenyl)nicotinonitrile,
2-amino-4-(4-bromophenyl)-6-methylnicotinonitrile,
2-amino-4-thien-2-yl-5,6,7,8,9,10-hexahydrocycloocta[b]pyridine-3-carbonitrile,
2-amino-4-(4-chlorophenyl)-6,7,8,9-tetrahydro-5H-cyclohepta[b]pyridine-3-carbonitrile,
2-(allylamino)-5-amino-7-(4-bromophenyl)thieno[3,2-b]pyridine-3,6-dicarbonitrile,
2-amino-4-pyridin-3-yl-5,6,7,8,9,10-hexahydrocycloocta[b]pyridine-3-carbonitrile,
2-amino-4-(4-bromophenyl)-6-tert-butylnicotinonitrile,
1-(2-furyl)-3-morpholin-4-yl-5,6,7,8-tetrahydroisoquinoline-4-carbonitrile,
2-amino-4-(4-methylphenyl)-6,7-dihydro-5H-cyclopenta[b]pyridine-3-carbonitrile,
2-amino-7,7-dimethyl-7,8-dihydro-5H-pyrano[4,3-b]pyridine-3-carbonitrile,
2-amino-6-isobutyl-4-(4-methoxyphenyl)nicotinonitrile,
4,6-diamino-2-oxo-1-phenyl-2,3-dihydro-1H-pyrrolo[2,3-b]pyridine-5-carbonitrile,
2-amino-4-(2-methoxyphenyl)-5,6-dimethylnicotinonitrile,
2-(dimethylamino)-4-(2-fluorophenyl)-6-(2-furyl)nicotinonitrile,
2-(dimethylamino)-4-(2-fluorophenyl)-6-(2-furyl)nicotinonitrile,
4-(2-fluorophenyl)-6-(2-furyl)-2-(methylamino)nicotinonitrile,
4-(2-fluorophenyl)-6-(2-furyl)-2-morpholin-4-ylnicotinonitrile,
tert-butyl N-[3-cyano-4-(2-fluorophenyl)-6-(2-furyl)pyridin-2-yl]glycinate,
2-(ethylamino)-4-(2-fluorophenyl)-6-(2-furyl)nicotinonitrile,
ethyl 4-[6-amino-5-cyano-4-(2-fluorophenyl)pyridin-2-yl]benzoate,
2-amino-6-(2-fluorophenyl)-4-(3-furyl)nicotinonitrile,
6-amino-4-(2-fluorophenyl)-2,2′-bipyridine-5-carbonitrile,
2-amino-4-(2-fluorophenyl)-6-thien-2-ylnicotinonitrile,
ethyl 6-amino-5-cyano-4-(2-fluorophenyl)pyridine-2-carboxylate,
2-amino-6-(2-furyl)-4-phenylnicotinonitrile,
ethyl 2-amino-3-cyano-4-(2-furyl)-5,6,7,8-tetrahydroquinoline-6-carboxylate,
2-amino-4-(2-furyl)-6-(4-hydroxyphenyl)-5-methylnicotinonitrile,
2-amino-4-(2-furyl)-6-(4-methoxyphenyl)-5-methylnicotinonitrile,
2-amino-6-(4-fluorophenyl)-4-(2-furyl)-5-methylnicotinonitrile,
2-amino-4-(2-furyl)-5,6-diphenylnicotinonitrile,
2-amino-4-(2-furyl)-5-methyl-6-phenylnicotinonitrile,
2-amino-6-(3,4-dimethylphenyl)-4-(2-furyl)nicotinonitrile,
2-amino-6-(4-fluorophenyl)-4-(2-furyl)nicotinonitrile,
2-amino-4-(3-fluorophenyl)-6-(3-hydroxyphenyl)nicotinonitrile,
6-amino-4-(3-fluorophenyl)-2,4′-bipyridine-5-carbonitrile,
6-amino-4-(2-fluorophenyl)-2,4′-bipyridine-5-carbonitrile,
2-amino-4-butyl-6-methylnicotinonitrile,
2-amino-6-methyl-4-propylnicotinonitrile,
2-amino-4-ethyl-6-methylnicotinonitrile, 2-amino-4,6-dimethylnicotinonitrile,
2-amino-4-[2-(hexyloxy)phenyl]-6,7-dihydro-5H-pyrazolo[3,4-h]quinoline-3-carbonitrile,
2-amino-4-[2-(beta-D-glucopyranosyloxy)phenyl]-6,7-dihydro-5H-pyrazolo[3,4-h]quinoline-3-carbonitrile,
4-[2-(allyloxy)phenyl]-2-amino-6,7-dihydro-5H-pyrazolo[3,4-h]quinoline-3-carbonitrile,
methyl [2-(2-amino-3-cyano-6,7-dihydro-5H-pyrazolo[3,4-h]quinolin-4-yl)phenoxy]acetate,
2-amino-4-(2-ethoxyphenyl)-6,7-dihydro-5H-pyrazolo[3,4-h]quinoline-3-carbonitrile,
ethyl 4-[2-amino-3-cyano-6-(2-furyl)pyridin-4-yl]-1H-pyrrole-2-carboxylate,
2-amino-6-methylnicotinonitrile,
2-amino-6-(4-cyanophenyl)-4-(2-furyl)nicotinonitrile,
2-amino-6-(4-fluorobenzyl)-4-(2-furyl)nicotinonitrile,
2-amino-5-(4-fluorophenyl)-4-(2-furyl)-6-methylnicotinonitrile,
2-amino-4-(2-furyl)-6-(4-methoxyphenyl)nicotinonitrile,
2-amino-4-(2-methylphenyl)-5,6,7,8-tetrahydroquinoline-3-carbonitrile,
2-amino-4-(4-methoxyphenyl)-5,6,7,8-tetrahydroquinoline-3-carbonitrile,
2-amino-4-phenyl-5,6,7,8-tetrahydroquinoline-3-carbonitrile,
2-amino-6-(4-methoxyphenyl)-4-(2-methylphenyl)nicotinonitrile,
2-amino-4,6-bis(4-methoxyphenyl)nicotinonitrile,
2-amino-4-(3-chlorophenyl)-6-(4-methoxyphenyl)nicotinonitrile,
2-amino-4-(2-chlorophenyl)-6-(4-methoxyphenyl)nicotinonitrile,
2-amino-4-(2-furyl)-5,6,7,8-tetrahydro-1,6-naphthyridine-3-carbonitrile,
2-amino-4-(2-furyl)-6-(4-methylphenyl)nicotinonitrile,
2-amino-4-(2-furyl)-6-phenylnicotinonitrile,
6-amino-4-(2-furyl)-2,3′-bipyridine-5-carbonitrile,
2-amino-6-(1,3-benzodioxol-5-yl)-4-(2-furyl)nicotinonitrile,
2-amino-4-isoquinolin-4-yl-6-(4-methoxyphenyl)nicotinonitrile,
2-amino-4-(1-benzothien-3-yl)-6-(4-methoxyphenyl)nicotinonitrile,
2-amino-6-(4-methoxyphenyl)-4-thien-3-ylnicotinonitrile,
2-amino-4-(3-furyl)-6-(4-methoxyphenyl)nicotinonitrile,
2-amino-6-(4-methoxyphenyl)-4-(1H-pyrrol-2-yl)nicotinonitrile,
2-amino-4-(2-furyl)-6-(1H-pyrrol-2-yl)nicotinonitrile,
2′-amino-6′-(4-methoxyphenyl)-3,4′-bipyridine-3′-carbonitrile,
2-amino-4-[2-(trifluoromethoxy)phenyl]-6,7-dihydro-5H-pyrazolo[3,4-h]quinoline-3-carbo nitrile,
2-amino-4-(2-furyl)-5H-thiochromeno[4,3-b]pyridine-3-carbonitrile,
2-amino-4-{4-[(2-cyanoethyl)(methyl)amino]phenyl}-6,7-dihydro-5H-pyrazolo[3,4-h]quinoline-3-carbonitrile,
2-amino-4-[2-(2-hydroxyethoxy)phenyl]-6,7-dihydro-5H-pyrazolo[3,4-h]quinoline-3-carbonitrile,
2-amino-4-(2-methylphenyl)-6,7-dihydro-5H-pyrazolo[3,4-h]quinoline-3-carbonitrile,
2-amino-4-[4-(dimethylamino)phenyl]-6,7-dihydro-5H-pyrazolo[3,4-h]quinoline-3-carbonit rile,
2-amino-4-(1H-indol-7-yl)-6,7-dihydro-5H-pyrazolo[3,4-h]quinoline-3-carbonitrile,
methyl 4-(2-amino-3-cyano-6,7-dihydro-5H-pyrazolo[3,4-h]quinolin-4-yl)benzoate,
methyl 2-(2-amino-3-cyano-6,7-dihydro-5H-pyrazolo[3,4-h]quinolin-4-yl)benzoate,
[2-(2-amino-3-cyano-6,7-dihydro-5H-pyrazolo[3,4-h]quinolin-4-yl)phenoxy]acetic acid,
2-amino-6-phenylnicotinonitrile,
2-amino-6-cyclohexylnicotinonitrile,
2-amino-4-(2-furyl)-6-(1-trityl-1H-pyrazol-4-yl)nicotinonitrile, and
2-amino-4-(2-fluorophenyl)-6-(4-hydroxyphenyl)nicotinonitrile,

8. The compound according to claim 1, wherein the aminocyanopyridine MK-2 inhibiting compound comprises at least one compound that is selected from the group consisting of:
2-amino-4-(2-fluorophenyl)-6,8-dihydro-5H-pyrazolo[3,4-h]quinoline-3-carbonitrile,
2-amino-4-(2-furyl)-6,7-dihydro-5H-pyrazolo[3,4-h]quinoline-3-carbonitrile,
2-amino-4-(2,3-difluorophenyl)-6,7-dihydro-5H-pyrazolo[3,4-h]quinoline-3-carbonitrile,
8-amino-6-(2-furyl)-4,5-dihydro-1H-pyrazolo[4,3-h]quinoline-7-carbonitrile,
2-amino-3-cyano-4-(2-furyl)-5,6-dihydrobenzo[h]quinoline-8-carboxylic acid,
4-[2-amino-3-cyano-6-(2-furyl)pyridin-4-yl]-1H-pyrrole-2-carboxamide,
2-amino-4-phenyl-6,8-dihydro-5H-pyrazolo[3,4-h]quinoline-3-carbonitrile,
2-amino-6-(2-furyl)-4-(1-methyl-1H-imidazol-4-yl)nicotinonitrile,
8-amino-6-(2-furyl)-4,5-dihydro-1H-pyrazolo[4,3-h]quinoline-7-carbonitrile,
2-amino-4-(2-furyl)-8-hydroxy-5,6-dihydrobenzo[h]quinoline-3-carbonitrile,
2-amino-4-(2,6-difluorophenyl)-6,7-dihydro-5H-pyrazolo[3,4-h]quinoline-3-carbonitrile,
2-amino-6-(4-hydroxyphenyl)-4-(1H-imidazol-5-yl)nicotinonitrile,
2-amino-4-(2-fluorophenyl)-6-(2-furyl)nicotinonitrile, 2-amino-4-(2-fluorophenyl)-6-(2-furyl)nicotinonitrile,
2-amino-4-(2-fluorophenyl)-5,6-dihydrobenzo[h]quinoline-3-carbonitrile,
4-[6-amino-5-cyano-4-(2-furyl)pyridin-2-yl]benzoic acid,
2-amino-6-(2-furyl)-4-(1H-imidazol-5-yl)nicotinonitrile,
2-amino-4-(2-furyl)-6-(1H-pyrazol-3-yl)nicotinonitrile,
2-amino-3-cyano-4-(4H-1,2,4-triazol-3-yl)-5,6-dihydrobenzo[h]quinoline-8-carboxylic acid,
2-amino-6-(3-hydroxyphenyl)-4-(1H-imidazol-5-yl)nicotinonitrile,
2-amino-6-(2-furyl)-4-(1H-imidazol-4-yl)nicotinonitrile,
2-amino-4-(2,4-difluorophenyl)-6,7-dihydro-5H-pyrazolo[3,4-h]quinoline-3-carbonitrile, 4,6-diamino-2-(trifluoromethyl)-2,3-dihydrofuro[2,3-b]pyridine-5-carbonitrile,
2-amino-4-(2-furyl)-6,8-dihydro-5H-pyrrolo[3,4-h]quinoline-3-carbonitrile,
4-[6-amino-5-cyano-4-(2-fluorophenyl)pyridin-2-yl]benzoic acid,
2-amino-4-(2-furyl)-5,6-dihydro-1,8-phenanthroline-3-carbonitrile,
2-amino-6-(3,4-dihydroxyphenyl)-4-(2-fluorophenyl)nicotinonitrile,
2-amino-4-(1-methyl-1H-imidazol-4-yl)-6-phenylnicotinonitrile,
2-amino-4-(2-furyl)-6-(1H-pyrazol-3-yl)nicotinonitrile,
4-[6-amino-5-cyano-4-(1H-imidazol-5-yl)pyridin-2-yl]benzoic acid,
2-amino-4-(3-fluorophenyl)-6,8-dihydro-5H-pyrazolo[3,4-h]quinoline-3-carbonitrile,
2-amino-6-(3,4-dihydroxyphenyl)-4-(2-fluorophenyl)nicotinonitrile,
N-{4-[6-amino-5-cyano-4-(2-furyl)pyridin-2-yl]phenyl}methanesulfonamide,
2-amino-4-(2-furyl)-6,7-dihydro-5H-pyrrolo[2,3-h]quinoline-3-carbonitrile,
2-amino-4-(1H-imidazol-5-yl)-6-phenylnicotinonitrile,
2-amino-4-(2-furyl)-5,6-dihydrobenzo[h]quinoline-3-carbonitrile,
2-amino-4-(1H-imidazol-5-yl)-6-(4-methoxyphenyl)nicotinonitrile,
2-amino-6-(3-chlorophenyl)-4-(1H-imidazol-5-yl)nicotinonitrile,
2-amino-4-(2-furyl)-6-(1H-pyrazol-4-yl)nicotinonitrile,
2-amino-4-(4-methoxyphenyl)-6,7-dihydro-5H-pyrazolo[3,4-h]quinoline-3-carbonitrile,
2-amino-4-(2,5-difluorophenyl)-6,7-dihydro-5H-pyrazolo[3,4-h]quinoline-3-carbonitrile,
2-amino-4-(4-fluorophenyl)-6,8-dihydro-5H-pyrazolo[3,4-h]quinoline-3-carbonitrile,
2-amino-4-(4H-1,2,4-triazol-3-yl)-5,6-dihydrobenzo[h]quinoline-3-carbonitrile,
4,6-diamino-2-(chloromethyl)-2,3-dihydrofuro[2,3-b]pyridine-5-carbonitrile,
2-amino-4-(1H-imidazol-4-yl)-6-phenylnicotinonitrile,
4-[6-amino-5-cyano-4-(2-furyl)pyridin-2-yl]benzenesulfonamide,
4-[6-amino-5-cyano-4-(2-furyl)pyridin-2-yl]phenylboronic acid,
2-amino-6-(4-methoxyphenyl)-4-(4H-1,2,4-triazol-3-yl)nicotinonitrile,
2-amino-4-(2-fluorophenyl)-6-(3-furyl)nicotinonitrile,
2-amino-6-(2-furyl)-4-(methylthio)nicotinonitrile,
2-amino-4-(2-fluorophenyl)-6-(3-hydroxyphenyl)nicotinonitrile,
8-amino-6-(2-furyl)-4,5-dihydro-2H-pyrazolo[4,3-h]quinoline-7-carbonitrile,
2-amino-4-(2-bromophenyl)-6-(2-furyl)nicotinonitrile,
2-amino-4-(2-fluorophenyl)-6-(4-hydroxyphenyl)nicotinonitrile,
2-amino-4-phenyl-6-thien-2-ylnicotinonitrile,
2-amino-4-(3-methoxyphenyl)-6,7-dihydro-5H-pyrazolo[3,4-h]quinoline-3-carbonitrile,
2-amino-4-(2-furyl)-7-methyl-6,7-dihydro-5H-pyrazolo[3,4-h]quinoline-3-carbonitrile,
2-amino-4-(2-fluorophenyl)-6-(1H-pyrrol-2-yl)nicotinonitrile,
2-amino-4-(2-furyl)-5-methyl-6,8-dihydro-5H-pyrazolo[3,4-h]quinoline-3-carbonitrile,
2-amino-4-(2-furyl)-6-(1-methyl-1H-pyrrol-3-yl)nicotinonitrile,
3-amino-5,6,7,8-tetrahydroisoquinoline-4-carbonitrile,
N-[4-(2-amino-3-cyano-6,7-dihydro-5H-pyrazolo[3,4-h]quinolin-4-yl)phenyl]acetamide,
6-amino-4-[(4-methoxyphenyl)amino]-2-(trifluoromethyl)-2,3-dihydrofuro[2,3-b]pyridine-5-carbonitrile,
4-[6-amino-5-cyano-4-(2-furyl)pyridin-2-yl]-N-(tert-butyl)benzenesulfonamide,
4,6-diamino-2-ethyl-2,3-dihydrofuro[2,3-b]pyridine-5-carbonitrile,
6-amino-4-(2-furyl)-2,4′-bipyridine-5-carbonitrile,
2,4-diamino-6-(methylthio)nicotinonitrile,
3-(2-amino-3-cyano-6,7-dihydro-5H-pyrazolo[3,4-h]quinolin-4-yl)benzoic acid,
2-amino-6-(4-chlorophenyl)-4-(1H-imidazol-5-yl)nicotinonitrile,
2-amino-4-(1,3-benzodioxol-4-yl)-6,7-dihydro-5H-pyrazolo[3,4-h]quinoline-3-carbonitrile, 4,6-diamino-2-methyl-2,3-dihydrofuro[2,3-b]pyridine-5-carbonitrile,
2-amino-4-(1H-imidazol-5-yl)-6-[4-(methylsulfonyl)phenyl]nicotinonitrile,
2,4-diaminoquinoline-3-carbonitrile,
2,8-diamino-4-(2-furyl)-5,6-dihydrobenzo[h]quinoline-3-carbonitrile,
2-amino-4,6-di(2-furyl)nicotinonitrile,
4,6-diamino-2-butyl-2,3-dihydrofuro[2,3-b]pyridine-5-carbonitrile,
ethyl 4-[6-amino-5-cyano-4-(1H-imidazol-5-yl)pyridin-2-yl]benzoate,
2,4-diamino-6-methoxynicotinonitrile,
2-amino-4-methylnicotinonitrile,
2-amino-4-(4-cyanophenyl)-6,7-dihydro-5H-pyrazolo[3,4-h]quinoline-3-carbonitrile,
2-amino-4-cyclopropyl-6-methylnicotinonitrile,
2-amino-4-(2-furyl)-6-(1-methyl-1H-pyrrol-2-yl)nicotinonitrile,
2-amino-4-(2-chlorophenyl)-6,7-dihydro-5H-pyrazolo[3,4-h]quinoline-3-carbonitrile,
2-amino-6-(2-furyl)-4-(4-phenoxyphenyl)nicotinonitrile,
2-amino-4-pyridin-3-yl-6,8-dihydro-5H-pyrazolo[3,4-h]quinoline-3-carbonitrile,
2-amino-6-{[2-(4-chlorophenyl)-2-oxoethyl]thio}-4-(2-furyl)pyridine-3,5-dicarbonitrile,
4-[2-amino-3-cyano-6-(2-furyl)pyridin-4-yl]phenylboronic acid,
2-amino-6-(3-chlorophenyl)-4-(1H-imidazol-4-yl)nicotinonitrile,
4-(6-amino-5-cyano-4-phenylpyridin-2-yl)-N-(tert-butyl)benzenesulfonamide,
2-amino-4-methoxynicotinonitrile,
4-[2-amino-3-cyano-6-(2-furyl)pyridin-4-yl]benzoic acid,
4,6-diamino-2-[(4-methoxyphenoxy)methyl]-2,3-dihydrofuro[2,3-b]pyridine-5-carbonitrile, 2-amino-4-(2-fluorophenyl)-6-(4-methoxyphenyl)nicotinonitrile,
4-[6-amino-5-cyano-4-(2-fluorophenyl)pyridin-2-yl]-N-tert-butyl)benzenesulfonamide, (2,4-diamino-3-cyano-5H-chromeno[2,3-b]pyridin-9-yl)oxy]acetic acid,
3-pyridinecarbonitrile, 2-amino-4-methylm
2-amino-6-(2-furyl)nicotinonitrile,
2-amino-4-(2-furyl)-6-(3-hydroxyphenyl)nicotinonitrile,
4-[6-amino-5-cyano-4-(2-furyl)pyridin-2-yl]benzamide,
2-amino-4-(2-furyl)-7-hydroxy-5,6-dihydrobenzo[h]quinoline-3-carbonitrile,
2-amino-4-(2-furyl)-6-(1H-indol-3-yl)nicotinonitrile,
2-amino-4-pyridin-4-yl-6,8-dihydro-5H-pyrazolo[3,4-h]quinoline-3-carbonitrile,
2-amino-4-(3-fluorophenyl)-6-(4-hydroxyphenyl)nicotinonitrile,
2-amino-4-[2-(difluoromethoxy)phenyl]-6,7-dihydro-5H-pyrazolo[3,4-h]quinoline-3-carbonitrile,
2-amino-4-(2-furyl)-6-thien-3-ylnicotinonitrile,
2-amino-4-(3-fluorophenyl)-6-(4-methoxyphenyl)nicotinonitrile,
2-[2-amino-3-cyano-6-(2-furyl)pyridin-4-yl]phenylboronic acid,
2,4-diamino-6-propylpyridine-3,5-dicarbonitrile, and prodrugs, salts, tautomers, and combinations thereof.

9. The compound according to claim 1, wherein the aminocyanopyridine MK-2 inhibiting compound comprises at least one compound that is selected from the group consisting of:
2-amino-4-(2-fluorophenyl)-6,8-dihydro-5H-pyrazolo[3,4-h]quinoline-3-carbonitrile,
2-amino-4-(2-furyl)-6,7-dihydro-5H-pyrazolo[3,4-h]quinoline-3-carbonitrile,
2-amino-4-(2,3-difluorophenyl)-6,7-dihydro-5H-pyrazolo[3,4-h]quinoline-3-carbonitrile,
8-amino-6-(2-furyl)-4,5-dihydro-1H-pyrazolo[4,3-h]quinoline-7-carbonitrile,
2-amino-3-cyano-4-(2-furyl)-5,6-dihydrobenzo[h]quinoline-8-carboxylic acid,
4-[2-amino-3-cyano-6-(2-furyl)pyridin-4-yl]-1H-pyrrole-2-carboxamide,
2-amino-4-phenyl-6,8-dihydro-5H-pyrazolo[3,4-h]quinoline-3-carbonitrile,
2-amino-6-(2-furyl)-4-(1-methyl-1H-imidazol-4-yl)nicotinonitrile,
8-amino-6-(2-furyl)-4,5-dihydro-1H-pyrazolo[4,3-h]quinoline-7-carbonitrile,
2-amino-4-(2-furyl)-8-hydroxy-5,6-dihydrobenzo[h]quinoline-3-carbonitrile,
2-amino-4-(2,6-difluorophenyl)-6,7-dihydro-5H-pyrazolo[3,4-h]quinoline-3-carbonitrile,
2-amino-6-(4-hydroxyphenyl)-4-(1H-imidazol-5-yl)nicotinonitrile,
2-amino-4-(2-fluorophenyl)-6-(2-furyl)nicotinonitrile,
2-amino-4-(2-fluorophenyl)-6-(2-furyl)nicotinonitrile,
2-amino-4-(2-fluorophenyl)-5,6-dihydrobenzo[h]quinoline-3-carbonitrile,
4-[6-amino-5-cyano-4-(2-furyl)pyridin-2-yl]benzoic acid,
2-amino-6-(2-furyl)-4-(1H-imidazol-5-yl)nicotinonitrile,
2-amino-4-(2-furyl)-6-(1H-pyrazol-3-yl)nicotinonitrile,
2-amino-3-cyano-4-(4H-1,2,4-triazol-3-yl)-5,6-dihydrobenzo[h]quinoline-8-carboxylic acid,
2-amino-6-(3-hydroxyphenyl)-4-(1H-imidazol-5-yl)nicotinonitrile,
2-amino-6-(2-furyl)-4-(1H-imidazol-4-yl)nicotinonitrile,
2-amino-4-(2,4-difluorophenyl)-6,7-dihydro-5H-pyrazolo[3,4-h]quinoline-3-carbonitrile, 4,6-diamino-2-(trifluoromethyl)-2,3-dihydrofuro[2,3-b]pyridine-5-carbonitrile,
2-amino-4-(2-furyl)-6,8-dihydro-5H-pyrrolo[3,4-h]quinoline-3-carbonitrile,
4-[6-amino-5-cyano-4-(2-fluorophenyl)pyridin-2-yl]benzoic acid,
2-amino-4-(2-furyl)-5,6-dihydro-1,8-phenanth roline-3-carbonitrile,
2-amino-6-(3,4-dihydroxyphenyl)-4-(2-fluorophenyl)nicotinonitrile,
2-amino-4-(1-methyl-1H-imidazol-4-yl)-6-phenylnicotinonitrile,
2-amino-4-(2-furyl)-6-(1H-pyrazol-3-yl)nicotinonitrile,
4-[6-amino-5-cyano-4-(1H-imidazol-5-yl)pyridin-2-yl]benzoic acid,
2-amino-4-(3-fluorophenyl)-6,8-dihydro-5H-pyrazolo[3,4-h]quinoline-3-carbonitrile,
2-amino-6-(3,4-dihydroxyphenyl)-4-(2-fluorophenyl)nicotinonitrile,
N-{4-[6-amino-5-cyano-4-(2-furyl)pyridin-2-yl]phenyl}methanesulfonamide,
2-amino-4-(2-furyl)-6,7-dihydro-5H-pyrrolo[2,3-h]quinoline-3-carbonitrile,
2-amino-4-(1H-imidazol-5-yl)-6-phenylnicotinonitrile,
2-amino-4-(2-furyl)-5,6-dihydrobenzo[h]quinoline-3-carbonitrile,
2-amino-4-(1H-imidazol-5-yl)-6-(4-methoxyphenyl)nicotinonitrile, and prodrugs, salts, tautomers, and combinations thereof.

10. The compound according to claim 1, wherein the aminocyanopyridine MK-2 inhibiting compound comprises at least one compound that is selected from the group consisting of:
2-amino-4-(2-fluorophenyl)-6,8-dihydro-5H-pyrazolo[3,4-h]quinoline-3-carbonitrile,
2-amino-4-(2-furyl)-6,7-dihydro-5H-pyrazolo[3,4-h]quinoline-3-carbonitrile,
2-amino-4-(2,3-difluorophenyl)-6,7-dihydro-5H-pyrazolo[3,4-h]quinoline-3-carbonitrile,
8-amino-6-(2-furyl)-4,5-dihydro-1H-pyrazolo[4,3-h]quinoline-7-carbonitrile, and prodrugs, salts, tautomers, and combinations thereof.

11. The compound according to claim 1, wherein the compound is capable of inhibiting the activity of mitogen activated protein kinase activated protein kinase-2.

12. The compound according to claim 1, having an MK-2 inhibition IC₅₀ of below 200 μM.

13. The compound according to claim 1, having an MK-2 inhibition IC₅₀ of below 10 μM.

14. The compound according to claim 1, having a TNFα release IC₅₀ value of below 200 μM in an in vitro cell assay.

15. The compound according to claim 1, having a TNFα release IC₅₀ values of below 5 μM in an in vitro cell assay.

16. The compound according to claim 1, wherein the aminocyanopyridine MK-2 inhibiting compound provides a degree of inhibition of TNFα in a rat LPS assay of at least about 25%.

17. The compound according to claim 1, wherein the aminocyanopyridine MK-2 inhibiting compound provides a degree of inhibition of TNFα in a rat LPS assay of above 80%.

18. A pharmaceutical composition comprising a pharmaceutically acceptable carrier and an aminocyanopyridine compound, or a pharmaceutically acceptable salt or tautomer or isomer thereof, the compound having the structure: 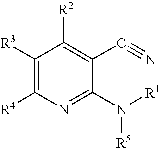
wherein:
R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-( C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;
R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy, 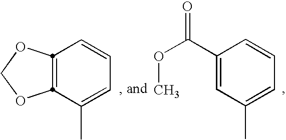
with the proviso that when R² is aryl, it is not substituted with nitro;
R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except that when R² is heteroaryl, R³ is other than cyano, and
where the R² and R³ groups are such that they optionally join to form a ring system selected from: 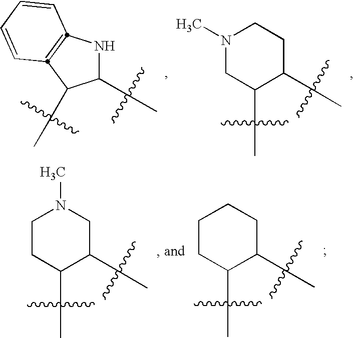
R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups optionally are substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy 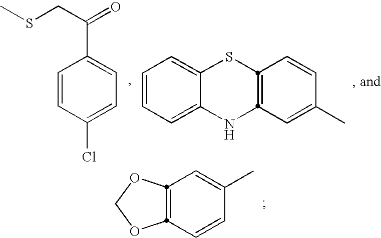
wherein the R³ and R⁴ groups are such that they optionally join to form a ring system selected from: 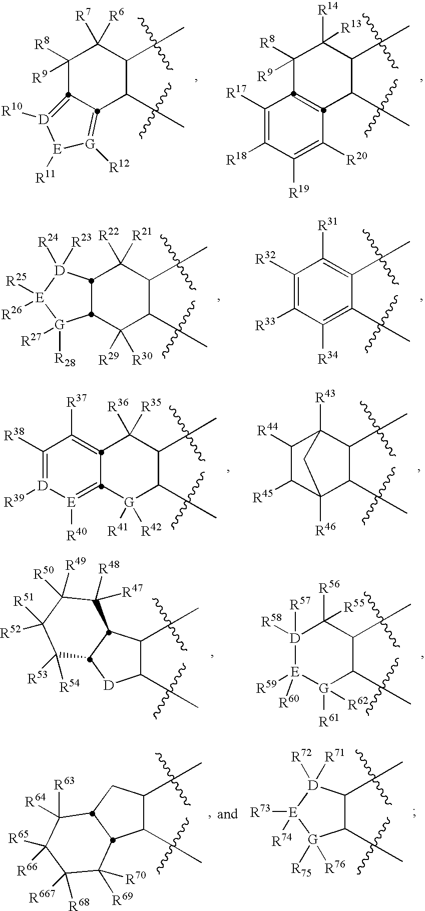
D, E and G are each independently selected from carbon, oxygen, sulfur, and nitrogen;
R⁵ is selected from the group consisting of —H, and C₁-C₅ alkyl, provided that at least one of R¹, R², R³, R⁴ and R⁵ is other than hydrogen; and
wherein the R¹ and R⁵ groups optionally join to form a piperidyl ring or a oxaxinyl ring;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵ and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl - ethyl ester, pyridyl C₁-C₄ alkyl, pyridyl C₁-C₄ alkoxy, —COO—CH₂—CH₃, with the proviso that when E is —N—, R³⁸ is not cyano, and that when G is —N—, R³⁶ is —H; and
wherein R³⁸ and R³⁹ are such that they optionally join to form a ring system of the type selected from: 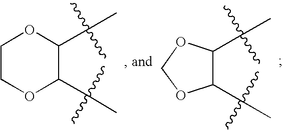
with the proviso that when R¹, R³ and R⁵ are hydrogen:
R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};
where Z₁ and Z₂ are each independently selected from the group consisting of hydrogen, alkoxycarbonyl, alkyl, alkylcarbonyl, benzyl, benzyloxycarbonyl, and formyl;
R^{A} is selected from the group consisting of aryl and arylalkyl;
R^{B} is selected from the group consisting of aryl, arylalkoxy, arylalkyl, aryloxy, heterocycle, and heterocyclealkyl; and
R⁴ is other than alkenyl, alkoxyalkynyl, alkyl, alkynyl, cycloalkyl, aryl, arylalkyl, heterocycle, heterocyclealkyl, or —R_{C}R_{D}R_{E};
where R_{C} is selected from the group consisting of aryl, arylalkyl, heterocycle and heterocyclealkyl;
R_{D} is selected from the group consisting of aryl, arylalkoxy, arylalkoxylmino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxylmino, heterocycleoxylminoalkyl, and heterocyclesulfonyl; and
R_{E} is absent or selected from the group consisting of aryl, arylalkoxy, arylalkoxylmino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxylmino, heterocycleoxylminoalkyl, and heterocyclesulfonyl.

19. A kit for the purpose of treating a TNFα mediated disease or disorder, the kit comprising a dosage form comprising at least one aminocyanopyridine compound, or a pharmaceutically acceptable salt thereof, the compound having the structure: 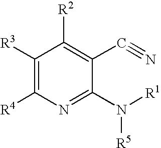
wherein:
R¹ is selected from the group consisting of —H, C₁-C₆ alkyl, C₂-C₆ alkenyl, C₂-C₆ alkynyl, carboxy C₁-C₄ alkyl, aryl C₁-C₄ alkyl, amino, amino C₁-C₄ alkyl, C₁-C₄ alkoxy, C₁-C₄ alkylamino, C₁-C₄ alkyl, di-(C₁-C₄ alkyl)amino C₁-C₄ alkyl, C₁-C₄ alkyl-C₁-C₄ alkyl, hydroxy C₁-C₄ alkyl, and aryl C₁-C₄ alkylcarbonyl;
R² is selected from the group consisting of —H, C₁-C₆ alkyl, C₂-C₆ alkenyl, C₂-C₆ alkynyl, amino, amino C₁-C₄ alkyl, C₁-C₄ alkylamino, aryl, heteroaryl, heterocyclyl, carboxy, carboxy C₁-C₄ alkyl, C₁-C₄ alkoxy, hydroxy, hydroxy C₁-C₄ alkyl, hydroxy C₁-C₄ alkylamino, hydroxy C₁-C₄ alkoxy, C₁-C₄ alkoxy C₁-C₄ alkyl, C₁-C₄ alkoxy C₁-C₄ alkylamino, amino C₁-C₄ alkylamino, aryl C₁-C₄ alkyl, C₁-C₄ alkylamino C₁-C₄ alkyl, di C₁-C₄ alkylamino C₁-C₄ alkyl, C₁-C₄ alkyl C₁-C₄ alkyl, carboxy C₁-C₄ alkyl, aryl C₁-C₄ alkylcarbonyl, phthaloamino C₁-C₄ alkyl, halo, carbamyl, C₁-C₄ alkylthio, C₁-C₄ alkoxyarylamino, C₁-C₁₀ mono- and bicyclic cycloalkyl, wherein aryl, heteroaryl, heterocyclyl, mono- and bicyclic cycloalkyl are optionally substituted with one or more of the groups selected from halogen, hydroxy, C₁-C₄ alkoxy, aryloxy, C₂-C₄ alkenyloxy, C₂-C₄ alkynyloxy, C₁-C₄ alkyl, carboxy, carbamyl, C₁-C₄ alkoxycarbonyl, C₁-C₄ alkoxycarbonyl C₁-C₄ alkoxy, carboxy C₁-C₄ alkoxyamino, C₁-C₄ alkylamino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, hydroxy C₁-C₄ alkoxy, halo C₁-C₄ alkoxy, tri-halo C₁-C₄ alkoxy, 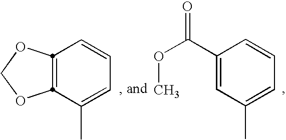
with the proviso that when R² is aryl, it is not substituted with nitro;
R³ is selected from the group consisting of —H, C₁-C₆ alkyl, C₂-C₆ alkenyl, C₂-C₆ alkynyl, cyano, amino C₁-C₄ alkyl, amino, aryl, wherein the aryl group is optionally substituted with one or more group selected from halogen, hydroxy, C₁-C₄ alkoxy, C₁-C₄ alkyl, carboxy, C₁-C₄ alkoxycarbonyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy, except that when R² is heteroaryl, R³ is other than cyano, and
where the R² and R³ groups are such that they optionally join to form a ring system selected from: 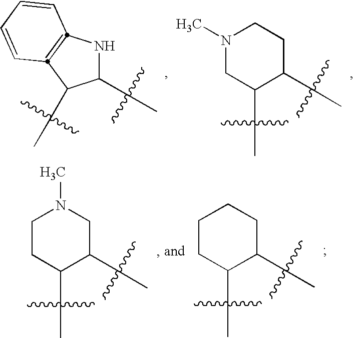
R⁴ is selected from the group consisting of —H, C₁-C₆ alkyl, C₂-C₆ alkenyl, C₂-C₆ alkynyl, hydroxy, C₁-C₄ alkylthio, C₁-C₄ alkoxy, C₁-C₄ alkoxycarbonyl, mercapto, N-imidazoylphenyl, C₁-C₄ isoalkyl, aminofluorobenzhydryl, aryl and heteroaryl, wherein the aryl and heteroaryl groups optionally can be substituted with one or more groups selected from halogen, hydroxy, C₁-C₄ alkoxy, C₁-C₄ alkyl, C₁-C₄ alkylthio, C₁-C₄ alkylsulfonyl, C₁-C₄ alkylsulfinyl, cartoxy, carbamyl, C₁-C₄ alkoxycarbonyl, carboxy C₁-C₄ alkyl, carboxy C₁-C₄ alkoxy, amino, di-C₁-C₄ alkylamino, N—C₁-C₄ alkyl-N-cyano C₁-C₄ alkylamino, nitro, C₁-C₄ alkylcarbonylamino, cyano, halo C₁-C₄ alkyl, di-halo C₁-C₄ alkyl, tri-halo C₁-C₄ alkyl, halo C₁-C₄ alkoxy, di-halo C₁-C₄ alkoxy, tri-halo C₁-C₄ alkoxy 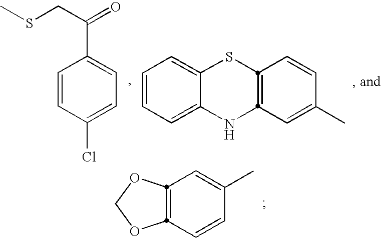
wherein the R³ and R⁴ groups are such that they can join to form a ring system selected from: 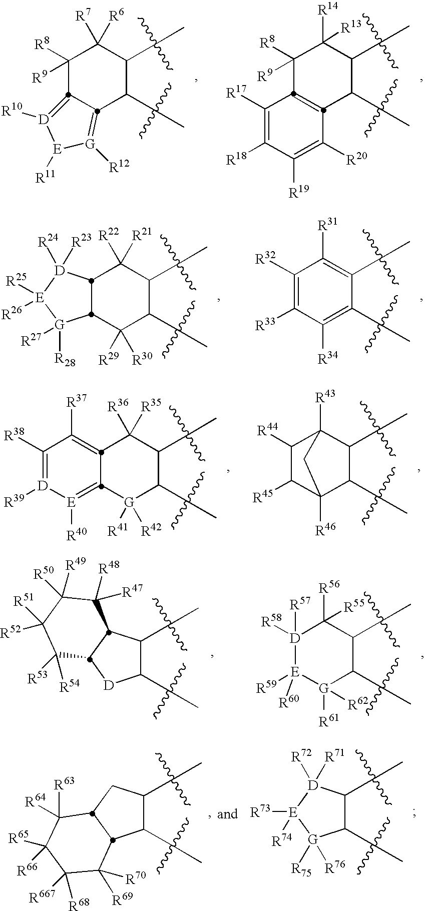
D, E and G are each independently selected from carbon, oxygen, sulfur, and nitrogen;
R⁵ is selected from the group consisting of —H, and C₁-C₅ alkyl, provided that at least one of R¹, R², R³, R⁴ and R⁵ is other than hydrogen; and
wherein the R¹ and R⁵ groups optionally join to form a piperidyl ring or a oxaxinyl ring;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵ and R⁷⁶ are each optionally present and are each independently selected from the group consisting of —H, C₁-C₄ alkyl, C₂-C₄ alkenyl, C₂-C₄ alkynyl, C₁-C₄ isoalkyl, amino, nitro, hydroxy, C₁-C₄ alkoxy, C₁-C₄ alkenoxy, oxo, carboxy, halo, halo C₁-C₄ alkyl, dihalo C₁-C₄ alkyl, trihalo C₁-C₄ alkyl, cyano, cyano C₁-C₄ alkyl, dicyano C₁-C₄ alkyl, halophenyl, hydroxy C₁-C₄ alkoxy, C₁-C₄ alkoxy C₁-C₄ alkoxy, —(CH₂)—O—(C₆H₄)—O—(CH₃), carboxy C₁-C₄ alkoxy, C₁-C₄ alkylcarboxy C₁-C₄ alkoxy, C₁-C₄ alkoxyamino, C₁-C₄ alkylamino, di C₁-C₄ alkylamino, tri C₁-C₄ alkylamino, amino C₁-C₄ alkoxy, diamino C₁-C₄ alkoxy, C₁-C₄ alkylamino C₁-C₄ alkoxy, di C₁-C₄ alkylamino C₁-C₄ alkoxy, cyano C₁-C₄ alkoxy C₁-C₄ alkyl, —(CH₂)—O—(CF₂)—CHF₂, tetra C₁-C₄ alkoxy C₁-C₄ alkyl, phenyl, benzyl, benzoyl, aryl, N-morpholinyl, morpholinyl C₁-C₄ alkoxy, pyrrolidyl C₁-C₄ alkoxy, N-pyrrolidyl C₁-C₄ alkoxy, C₁-C₄ alkylcarboxy, carboxy C₁-C₄ alkyl - ethyl ester, pyridyl C₁-C₄ alkyl, pyridyl C₁-C₄ alkoxy, —COO—CH₂—CH₃, with the proviso that when E is —N—, R³⁸ is other than cyano, and that when G is —N—, R³⁶ is —H; and
wherein R³⁸ and R³⁹ are such that they optionally join to form a ring system of the type selected from: 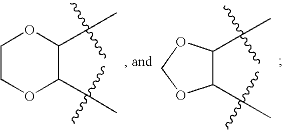
with the proviso that when R¹, R³ and R⁵ are hydrogen:
R² is other than alkenyl, alkyl, alkynyl, aryl, arylalkyl, cycloalkyl, cycloalkylalkyl, heterocycle, heterocyclealkyl, heterocyclealkylcarbonyl, (NZ₁Z₂)alkyl, or —R_{A}R_{B};
where Z₁ and Z₂ are each independently selected from the group consisting of hydrogen, alkoxycarbonyl, alkyl, alkylcarbonyl, benzyl, benzyloxycarbonyl, and formyl;
R^{A} is selected from the group consisting of aryl and arylalkyl;
R^{B} is selected from the group consisting of aryl, arylalkoxy, arylalkyl, aryloxy, heterocycle, and heterocyclealkyl; and
R⁴ is other than alkenyl, alkoxyalkynyl, alkyl, alkynyl, cycloalkyl, aryl, arylalkyl, heterocycle, heterocyclealkyl, or —R_{C}R_{D}R_{E};
where R_{C} is selected from the group consisting of aryl, arylalkyl, heterocycle and heterocyclealkyl;
R_{D} is selected from the group consisting of aryl, arylalkoxy, arylalkoxylmino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxylmino, heterocycleoxylminoalkyl, and heterocyclesulfonyl; and
R_{E} is absent or selected from the group consisting of aryl, arylalkoxy, arylalkoxylmino, arylalkyl, aryloxy, heterocycle, heterocyclealkoxy, heterocyclealkyl, heterocyclecarbonyl, heterocycleimino, heterocycleoxy, heterocycleoxyalkyl, heterocycleoxylmino, heterocycleoxylminoalkyl, and heterocyclesulfonyl.
